(19) United States
(12) Patent Application Publication
(10) Pub. No.: US 2025/0058002 A1
(43) Pub. Date: Feb. 20, 2025

(54) ELECTROCHEMICAL INHIBITION OF REDOX ACTIVE BACTERIA AND RELATED DEVICES, METHODS AND SYSTEMS

(71) Applicants: CALIFORNIA INSTITUTE OF TECHNOLOGY, Pasadena, CA (US); NAVAL RESEARCH LABORATORY, Washington, DC (US)

(72) Inventors: Dianne K. NEWMAN, Pasadena, CA (US); Leonard TENDER, Bethesda, MD (US)

(21) Appl. No.: 18/646,571

(22) Filed: Apr. 25, 2024

Related U.S. Application Data
(60) Provisional application No. 63/461,855, filed on Apr. 25, 2023.

(51) Int. Cl. A61L 2/03; A61K 31/7036; A61N 1/04; A61P 31/04

(52) U.S. Cl. CPC A61L 2/035; A61K 31/7036; A61N 1/0468; A61P 31/04

(57) ABSTRACT

Provided herein are methods and systems and related devices and compositions for electrochemical control of viability of redox active bacteria. The electrochemical control is performed by applying to a working electrode contacting a medium known or suspected to comprise the redox active bacteria, a reducing potential which is lower of the midpoint potential of a redox active compound produced by the redox active bacteria.

### CROSS-REFERENCE TO RELATED APPLICATION

The present application claims priority to U.S. Provisional Application No. 63/461,855 entitled “Electrochemical Potentiation of Antibiotic Killing of Pseudomonas Aeruginosa Biofilms” filed Apr. 25, 2023, with docket number CIT 8997-P the content of which is incorporated herein by reference in their entirety. The present applications may also be related to U.S. Non-Provisional application Ser. No. 15/466,839 entitled “Phenazine Degrading Agents And Related Compositions, Methods And Systems For Interfering With Viability Of Bacteria” filed on Mar. 22, 2017, and granted on Feb. 9, 2019, with U.S. Pat. No. 10,913,936, and to U.S. Non-Provisional application Ser. No. 17/520,345 filed on Nov. 5, 2021, published as US 2022-0175892, the content of each of which is herein also incorporated herein by reference in its entirety.

### STATEMENT OF GOVERNMENT GRANT

This invention was made with government support under Grant No. AI127850 awarded by the National Institutes of Health. The government has certain rights in the invention.

### FIELD

The present disclosure relates to methods and systems for interfering with the viability of bacteria and related compounds and compositions. In particular, the present electrochemical inhibition of redox active and related agents, devices, compositions, methods and systems for interfering with the viability of the redox active bacteria.

### BACKGROUND

Redox active bacteria are bacteria which produce redox active compounds as part of their physiology and are important compound for their viability.

Whether for medical application or for fundamental biology studies, several methods are commonly used for the detection of and interference with the viability of bacteria. In particular when aimed at therapeutic or diagnostic applications.

Methods, systems and compositions have been developed to interfere with, and in particular, to reduce bacterial viability of redox active bacteria. However, development of more efficacious inhibition viability of redox active bacteria is still particularly challenging.

### SUMMARY

Provided herein, is an electrochemical approach to inhibition of the viability of redox active bacteria, as well as related devices, compositions, methods and systems that in several embodiments are configured for effective reduction of antibiotic resistance and/or survivability of redox active bacteria.

According to a first aspect, a method is described to decrease viability of redox active bacteria in a medium, the redox active bacteria producing a redox active compound having an oxidized state and a reduced state, the redox active compound further having a midpoint potential, the method comprising:
contacting the medium with
   a working electrode having a working electrode potential compared to a reference electrode and
   a counter electrode having a counter electrode potential compared to the reference electrode;
operating a voltage source to apply to the working electrode and the counter electrode a reducing voltage selected so that the working electrode potential has a reducing potential lower than the midpoint potential of the redox active compound,
the operating performed for a time and under conditions to increase the concentration of the redox active compound in the reduced state in the medium thus inhibiting viability of the redox active bacteria and preferably
   contacting the redox active bacteria with one or more antibiotic and/or additional antimicrobials when the concentration of the redox active compound in a reduced state is higher than the concentration of the redox active compound in an oxidized state.

According to a second aspect a system is described to decrease viability of a target redox active bacteria in a medium, the target redox active bacteria producing a target redox active compound having oxidized state and a reduced state, the target redox active compound further having a target midpoint potential the system comprising:
a voltage source operatively connected to a working electrode a counter electrode and a reference electrode, the voltage source configured to apply to the working electrode a working electrode potential relative to the reference potential lower than the target midpoint potential;
the system further comprising a look-up table reporting a set of redox active bacteria each accompanied by corresponding redox active compounds and midpoint potentials, a set of operating conditions, the redox active bacteria, redox active compound and related midpoint potentials comprising the target redox active bacteria, the target redox active compound and the target midpoint potentials; and
an antibiotic and/or other antimicrobial
for simultaneous combined or sequential use in the method to decrease viability of the of the redox active bacteria of the present disclosure.

According to a third aspect a device for decreasing viability of a target redox active bacteria in a medium is described, the target redox active bacteria producing a target redox active compound having oxidized state and a reduced state, the target redox active compound further having a target midpoint potential the device comprising:
a voltage source a working electrode a counter electrode and a reference electrode configured to present the working electrode for contact with a target area of the medium known or expected to comprise the target redox bacteria (if any);
the voltage source, configured to operate with the working electrode the counter electrode and the reference electrode, and to provide a reduction potential of the wording electrode relative to the reference electrode, lower than the target midpoint potential which increases the concentrations of the redox active bacteria to a reduced state in the medium.

In preferred embodiments the device further comprises
at least one antimicrobial source, configured to release one or more antibiotic and/or other antimicrobials when the concentration of the redox active compound in the reduced state is higher than the concentration of the redox active compound in an oxidized state. In particular in some embodiments the release is timed to occur before, simultaneously or after the working electrode potential is lower than the midpoint potential depending on the experimental design.

The electrochemical inhibition and related devices, compositions, methods and systems herein described, in several embodiments are expected to be particularly effective in treating and/or prevent bacterial infection in vitro or in vivo.

In particular the electrochemical inhibition and related devices, compositions, methods and systems herein described, in several embodiments result an effective inhibition of bacteria viability which render the bacteria particularly susceptible to the action of antimicrobials.

More particularly the electrochemical inhibition and related devices, compositions, methods and systems herein described, in several embodiments allow killing of bacteria using antibiotics and/or additional antimicrobials in synergic combination with the application of a difference in voltage to the medium where the bacteria are known to be or expected to be present.

Accordingly, the electrochemical inhibition and related devices, compositions, methods and systems herein described, can be used in some embodiments, in combination with concentration of antibiotics below the minimum inhibitory concentration (MIC) while resulting in an effective killing of target redox bacteria in a medium.

The electrochemical inhibition and related devices, compositions, methods and systems herein described, in several embodiments, is expected to allow treatment and/or prevent progression of chronic infections such as diabetic foot ulcers and other infections where conventional approaches fail to successfully kill the underlying pathogenic bacteria.

The electrochemical inhibition and related devices, compositions, methods and systems herein described, in several embodiments, targets bacterial cells which that are in a physiological state that typically tolerates conventional drugs. The synergy between the electrochemical inhibition and the antimicrobial treatment demonstrated in the experimental section over a total population show that the stress of blocking the electro active species' of a redox bacteria can be successful in killing bacterial cells unresponsive to antibiotics and/or other antimicrobials.

The electrochemical inhibition and related devices, compositions, methods and systems herein described, in several embodiments, allow to successfully treat bacteria in biofilm formation by blocking the electro active species' of a redox bacteria involved in the bacteria respiration under anoxic or hypoxic conditions.

The electrochemical inhibition and related devices, compositions, methods and systems herein described, can be used in connection with applications wherein reduction of viability of redox active bacteria and/or reduction of antibiotic resistance is desired, which include but are not limited to medical application, drug research, biological analysis and diagnostics including but not limited to clinical applications. Additional exemplary applications include uses of the methods and system and related compositions herein described in several fields including basic biology research, applied biology, bio-engineering, etiology, medical research, medical diagnostics, therapeutics, and in additional fields identifiable by a skilled person upon reading of the present disclosure.

### BRIEF DESCRIPTION OF THE DRAWINGS

The accompanying drawings, which are incorporated into and constitute a part of this specification, illustrate one or more embodiments of the present disclosure and, together with the detailed description and the examples, serve to explain the principles and implementations of the disclosure.

FIGS. 1A to 1D report results of experiments showing that EET impacts cell survival and biofilm morphology. In particular, FIG. 1A shows a schematic representation of experimental setup. Working electrodes served as biofilm attachment surfaces that were at open circuit (neither oxidizes nor reduces PYO) or poised at the PYO-oxidizing potential of +100 mV or at the PYO-reducing potential of −400 mV vs Ag/AgCl. Growth reactors were incubated under oxic conditions for 5 d with fresh medium exchanged every 24 h. The working electrodes were then transferred to anoxic survival reactors that were flushed with N₂ gas and incubated for 72 h before harvesting and processing biofilm. WE, working electrode; CE, counter electrode; RE, reference electrode. FIG. 1B shows a schematic representation of redox cycling of PYO between cells that reduce PYOₒₓ to PYO_{red} and the electrode under PYO-oxidative (+100 mV) and PYO-reductive (−400 mV) conditions; under PYO-oxidative conditions, PYO_{red} is oxidized to PYOₒₓ, allowing redox cycling to proceed; under PYO-reductive conditions, PYOₒₓ is reduced to PYO_{red}, thereby breaking the cycle. FIG. 1C shows CFUs after 72 h under anoxic conditions (n=3) normalized to parallel OC negative control samples (see raw data in FIG. 2) for wild type, Δphz*, and Δphz*+10 μM PYO biofilms. Error bars represent standard error. FIG. 1D shows Fluorescence microscopy images of biofilm interface with electrode surface using TOTO-1 (cell-impermeable, eDNA) and Syto60 (cell-permeable, all DNA) from samples representative of three fields of view from triplicate cultures. Bar=50 μm. FIG. 1E is Vertical slice of complete z-stack along yellow dotted lines shown in wild-type panels in (D).

FIGS. 2A and 2B report results of experiments showing that reduced PYO acts synergistically with antibiotic treatment. In particular, FIG. 2A CFUs after 72 h under anoxic conditions normalized to parallel OC samples (see raw data in FIG. 6) in the presence of either 4 μg/mL gentamicin (Gent), 1 μg/mL meropenem (Mer), 4 μg/mL ciprofloxacin (Cir), or 10 μg/mL colistin (Col), n=3. Error bars represent standard error. Data from samples not treated with antibiotics (No abx) from FIGS. 1A to 1D plotted again for ease of comparison. FIG. 2B shows fluorescence microscopy images of biofilm interface with electrode surface using TOTO-1 (cell-impermeable, eDNA) and Syto60 (cell-permeable, all DNA) from representative samples shown in (A). Bar=50 μm.

FIG. 3: Electrochemical signal of wild type, Δphz*, and Δphz*+10 μM PYO showing PYO (E_{1/2}=−250 mV vs Ag/AgCl) is the only redox active signal observed under these conditions and is retained by wild type biofilms. Square wave voltammograms acquired immediately after transfer to anoxic reactors.

FIG. 4: Un-normalized data from FIGS. 1A-1E. CFUs after 72 hours under anoxic conditions, n=3. Box plots represent standard error.

FIG. 5: Cell survival within biofilms exposed to PYO-reductive or PYO-oxidative conditions at a distance of ˜3 cm. CFUs from biofilms grown on electrodes poised at OC shown in gray, poised at +100 mV (line) and unpoised (dashed) within the same reactor shown in teal, and poised at −400 mV (line) and unpoised (dashed) within same reactor shown in black.

FIG. 6: Un-normalized data from FIGS. 2A-2B. Panel A) CFUs after 72 hours under anoxic conditions in the presence of either 4 μg/ml gentamicin (Gent), 1 μg/ml meropenem (Mer), 4 μg/ml ciprofloxacin (Cir), or 10 μg/ml colistin (Col), n=3. Error bars represent standard error. Data from samples not treated with antibiotics (No abx) from FIG. 1 plotted again for ease of comparison. Panel B) Fluorescence microscopy images of OC samples at biofilm interface with electrode surface using TOTO-1 (cell-impermeable, eDNA) and Syto60 (cell-permeable, all DNA) from representative samples shown in A). Bar=50 um.

FIG. 7: Cell survival as a function of time. t=0 corresponds to transfer to anaerobic reactors and plotted at t=−10 are CFU counts for week-old aerobic biofilms prior to transfer. CFUs/cm² shown for parallel LB O₂ (line), which were incubated aerobically, and LB NO3 (dashed), which were incubated inside an anerobic chamber. CFUs from aerobic biofilms were collected and plated aerobically. Data shown from n=3.

FIG. 8: Toxicity of reduced PYO to liquid-grown cells. Exposure of mid-log aerobic P. aeruginosa PA14 liquid cultures to 1, 10, and 100 μM reduced PYO under anoxic conditions. Triplicate data shown normalized to untreated samples with line at mean.

FIG. 9: a timing diagram during the application of electric potential and treatment of antibiotics.

FIG. 10A shows an example bottom side of a bandage with two electrodes to deliver a voltage, one reference electrode, and one antibiotic releasing chip.

FIG. 10B shows an example top side of a bandage showing wired connections for the bandage of FIG. 10A.

FIG. 10C shows an example bottom side of a bandage incorporating an array of electrodes to provide an anti-bacterial effect.

FIG. 11 provides an example schematic showing configuration of electrodes in relation to medical equipment and position of a patient using electrosurgery, or for other sanitizing uses.

FIG. 12 shows an example schematic representation of embedding of electrodes in a medical implant.

FIG. 13 shows an example schematic representation of an aerator device which can be modified according to the indication of the present disclosure to include electrodes configured to apply a difference in voltage to disinfect the aerator itself and/or the liquid contacting the aerator.

FIG. 14 shows an example combination reference and counter electrode.

FIG. 15 shows an example combination counter and working electrode.

FIG. 16 shows Pourbaix diagram for water, showing lower practical potential limit (vs. NHE) from[1]).

### DETAILED DESCRIPTION

Provided herein, is an electrochemical approach to inhibition viability of redox active bacteria, as well as related devices, methods, systems and compositions that in several embodiments are configured for effective reduction of antibiotic resistance and/or survivability of phenazine producing bacteria.

The term “redox active” bacteria as used herein refer to bacteria which produce redox-active compound involved in various physiological processes that are crucial for their survival and function of the bacteria.

As used herein, the term “redox-active compound” refers to a chemical compound that is able to undergo reversible electrochemical conversion between an oxidation state and reduction state. A redox-active compound includes naturally occurring redox-active molecules, organic, inorganic or metal ion complexes. [2]

In particular, unless otherwise indicated, the term “redox-active compound” as used herein indicates a bacterially produced redox active compound which is produced by a bacteria as part of the physiology pf the bacteria.

Redox active compounds have very important role in the physiology and survival of many bacterial cells as will be understood by a skilled person. In particular, electron transfer reactions are fundamental to metabolism, for example in connection with the issue of energy generation (see e.g. photosynthesis and respiration) as will be understood by a skilled person. Redox active compounds can be involved in metabolic pathways in various stages of the bacterial life such for example in connection biofilm formation where extracellular electron transfer may be essential for survival of the bacteria. [2]

For example, metabolic reactions can occur where the final electron acceptor is a freely diffusible gas or a readily soluble species that the cell can easily access, or metal reducers face the task of transferring electrons to a solid form.

Exemplary redox active compounds comprise compounds from the Table 1 and Table 2 from Hernandez and Newman 2001 [2]

TABLE 1
[2]

| Redox potentials | E [symbol] (mV) | Reference |
|---|---|---|
| Alcaligin acid ox/red | −588 | 39 |
| NAD [symbol] /NADH | −320 | 26 |
| S [symbol] /HS⁻ | −270 | 26 |
| 2,6-AQDS/2,6-AHDS | −184 | 33 |
| Humics | −200 to +300 | 35 |
| ½ α · Fe [symbol] O [symbol] /Fe [symbol] | −159 | ^{a} |
| α · FeOOH/Fe [symbol] | −88 | ^{a} |
| Menaquinone ox/red | −75 | 26 |
| ACNQ ox/red | −71 | 42 |
| Pyocyanin ox/red | −34 | 49 |
| am · Fe(OH) [symbol] /Fe [symbol] | +59 | ^{a} |
| Ubiquinone ox/red | +113 | 26 |
| O₂/H₂O | +820 | 26 |

| Iron solubility and chelation | | log K | Reference |
|---|---|---|---|
| Alcaligin | Fe [symbol] L [symbol] = 2Fe [symbol] + 3L [symbol] | −64.8 | 39 |
| Goethite | α · FeOOH [symbol] + H₂O = Fe [symbol] + 3OH⁻ | −41.5 | 37 |
| Ferric | am · Fe(OH) [symbol] = Fe [symbol] + 3OH⁻ | −38.8 | 37 |
| hydroxide | | | |
| Hematite | ½α · Fe [symbol] O [symbol] + [symbol] H [symbol] O = Fe [symbol] + 3OH⁻ | −42.7 | 37 |
| Siderite | FeCO [symbol] = Fe [symbol] + CO [symbol] | −10.7 | 37 |
| Pyrrhotite | FeS [symbol] = Fe [symbol] + S [symbol] | −18.1 | 37 |
| Vivianite | Fe [symbol] (PO [symbol] ) [symbol] · 8H [symbol] O [symbol] = 3Fe [symbol] + 2PO [symbol] + | −36.0 | 37 |
| | 8H [symbol] O | | |

^{a} Redox potentials were calculated from Morel and Hering [37] assuming [Fe [symbol] ] = 1 μM. [symbol]
indicates data missing or illegible when filed

where E12 is determined vs NHE at pH7.

TABLE 2[2]

| Name | Structure | Function | Reference |
|---|---|---|---|
| Putrebactin | 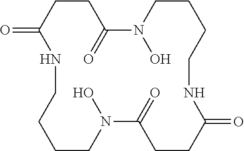 [SMILES: O=C1CCC(=O)N(O)CCCCNC(=O)CCC(=O)N(O)CCCCN1] | siderophore | 39 |
| 3-OC₆-HSL | 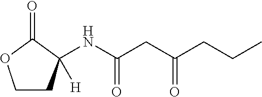 | signal | 53 |
| Quinotone | 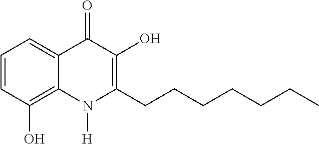 [SMILES: CCCCCCCc1[nH]c2c(O)cccc2c(=O)c1O] | signal | 58 |
| ACNQ | 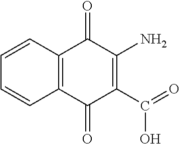 [SMILES: NC1=C(C(=O)O)C(=O)c2ccccc2C1=O] | redox shuttle | 42 |
| 2.6-AQDS | 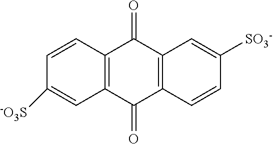 | redox shuttle | 32 |
| Pyocyanin | 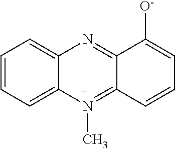 | antibiotic iron acquisition redox shuttle | 45, 47, 48 |
| P-2,6-bT | 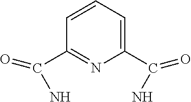 [SMILES: NC(=O)c1cccc(C(N)=O)n1] | siderophore (?) redox shuttle | 36 |
| Menaquinone | 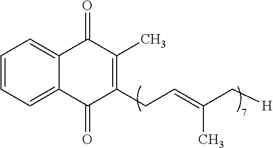 | redox shuttle | 26 |

Accordingly, redox-active compounds which are involved in bacterial processes can have a midpoint potential from +200 mV≤E_{1/2}> to −600 mV vs Ag/AgCl in an aqueous environment at pH7 as will be understood by a skilled person. In particular redox-active compounds can have a midpoint potential from +138 mV (midpoint potential of Fe(OH)3/Fe2) to −600 mV (midpoint potential for NADH/NAD+) vs Ag/AgCl in an aqueous environment at pH7 as will also be understood by a skilled person.

Redox active compounds produced by redox active bacteria comprise redox active quinones which are involved in energy production, metabolism, gene regulation, stress resistance, interspecies interactions, and environmental adaptation of bacterial cells producing them [3][4][5]. In particular, redox active quinones play a significant role in the electron transport chain, essential for cellular respiration and energy production and also enable bacteria to adapt to a range of environmental conditions, thereby enhancing their survival and functionality (see e.g.) [6].

Accordingly, redox active compounds in the sense of the disclosure comprise compounds of formula (I)

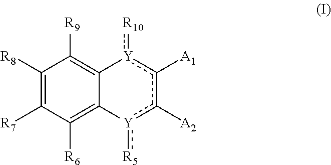

in which Y is C or N,
   wherein when Y is C,
      R5 and R10 are O
      A1 is an alkyl or substituted alkyl, alkenyl, alkynyl, aryl, heteroaryl acyl group, or COOH
      A2 is H or an alkyl or substituted alkyl, alkenyl, alkynyl, aryl, heteroaryl acyl group or NH2, and
      R6, R7, R8 and R9 are H,
   wherein when Y is N
      R5 and R10 are independently null, H, an alkyl group, an acyl group, or O
      R6, R7, R8 and R9 are independently H, O, OH, COOH, C(O)H, NH₂, SH, Cl, Br, SO₃H, alkoxy, OC(O)-alkyl, C(O)O-alkyl, C(O)NH-alkyl, C(O)N-(alkyl)₂, S-alkyl, or an alkyl, substituted alkyl, alkenyl, alkynyl, aryl, heteroaryl or acyl group
      and A1 and A2 are joined together to form a moiety of structure (1a)

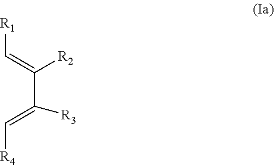

in which R1, R2, R3, R4 are independently H, O, OH, COOH, C(O)H, NH₂, SH, Cl, Br, SO₃H, alkoxy, OC(O)-alkyl, C(O)O-alkyl, C(O)NH-alkyl, C(O)N-(alkyl)₂, S-alkyl, or an alkyl, substituted alkyl, alkenyl, alkynyl, aryl, heteroaryl or acyl group
and wherein the compound has a midpoint potential E_{1/2} such that +200 mVS≤E_{1/2}> to −600 mV vs Ag/AgCl in an aqueous environment at pH7.

The compounds of Formula (I) are inclusive of all tautomer forms (e.g. R5=Me, R1=O with R10=null; also R5=Me and R1=OH with R10=null; and R5=Me with N charged, R1=O—) with R10=H) as will be understood by a skilled person.

The compound of Formula (I) can also have a midpoint potential E_{1/} ranging from +138 mv to −517 mV 0 mVS E_{1/2}> to −600 mV, from 0 to −517 mV, from 0 to −500 mV; from 0 to −400 mV; from −100 to −400 mV; from −125 to −375 mV vs Ag/AgCl in an aqueous environment at pH7.

Exemplary redox active quinones produced by redox active bacteria comprise

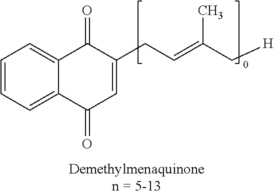

produced by Enterococcus faecium;

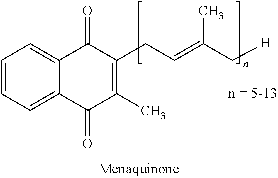

produced by Staphylococcus aureus, Klebsiella pneumoniae, Enterobacter spp. and Escherichia coli, and

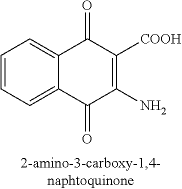 [SMILES: NC1=C(C(=O)O)C(=O)c2ccccc2C1=O]

produced by Propionibacterium freundenreichii and Bifidobacterium longum, and additional compounds identifiable by a skilled person upon reading of the present disclosure.

In some embodiments the redox active compounds are phenazines and the corresponding bacteria are phenazine producing bacteria.

The term “phenazine” as used herein indicates small, colorful, redox-active compounds formed by bacteria to perform diverse physiological functions. In particular, “phenazines” in the sense of the disclosure comprise several phenazines of bacterial origin produced by bacteria such as Pseudomonas spp., Streptomyces spp., Burkholderia spp., and Pantoea agglomerans. The absorption spectra of phenazines are characteristic, with an intense peak in the range 250-290 nm and a weaker peak at 350-400 nm. At least one main band occurs in the visible region (400-600 nm) to which the phenazines owe their colors. Phenazines in the sense of the disclosure comprise compounds of Formula (I):

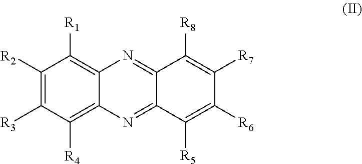

where R₁—R₈ are independently selected from hydrogen, hydroxy, alkoxy, alkyl, alkenyl, alkynyl, aryl, heteroaryl, acyl, and other groups identifiable to the skilled person.

Additionally, phenazines can include, but are not limited to, molecules according to the structures and formulas below:

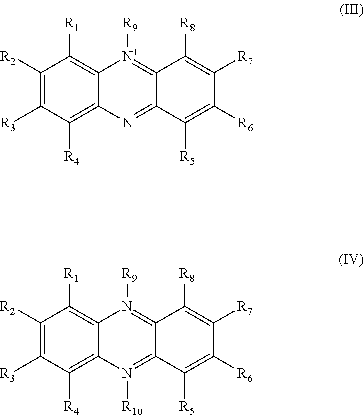

where R₁—R₁₀ are independently selected from hydrogen, hydroxy, alkoxy, alkyl, alkenyl, alkynyl, aryl, heteroaryl, acyl, and other groups identifiable to the skilled person, and one of R₁—R₁₀ is a negatively charged substituent (formal charge of −1) such as

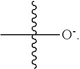

In particular, exemplary phenazine structures comprise:

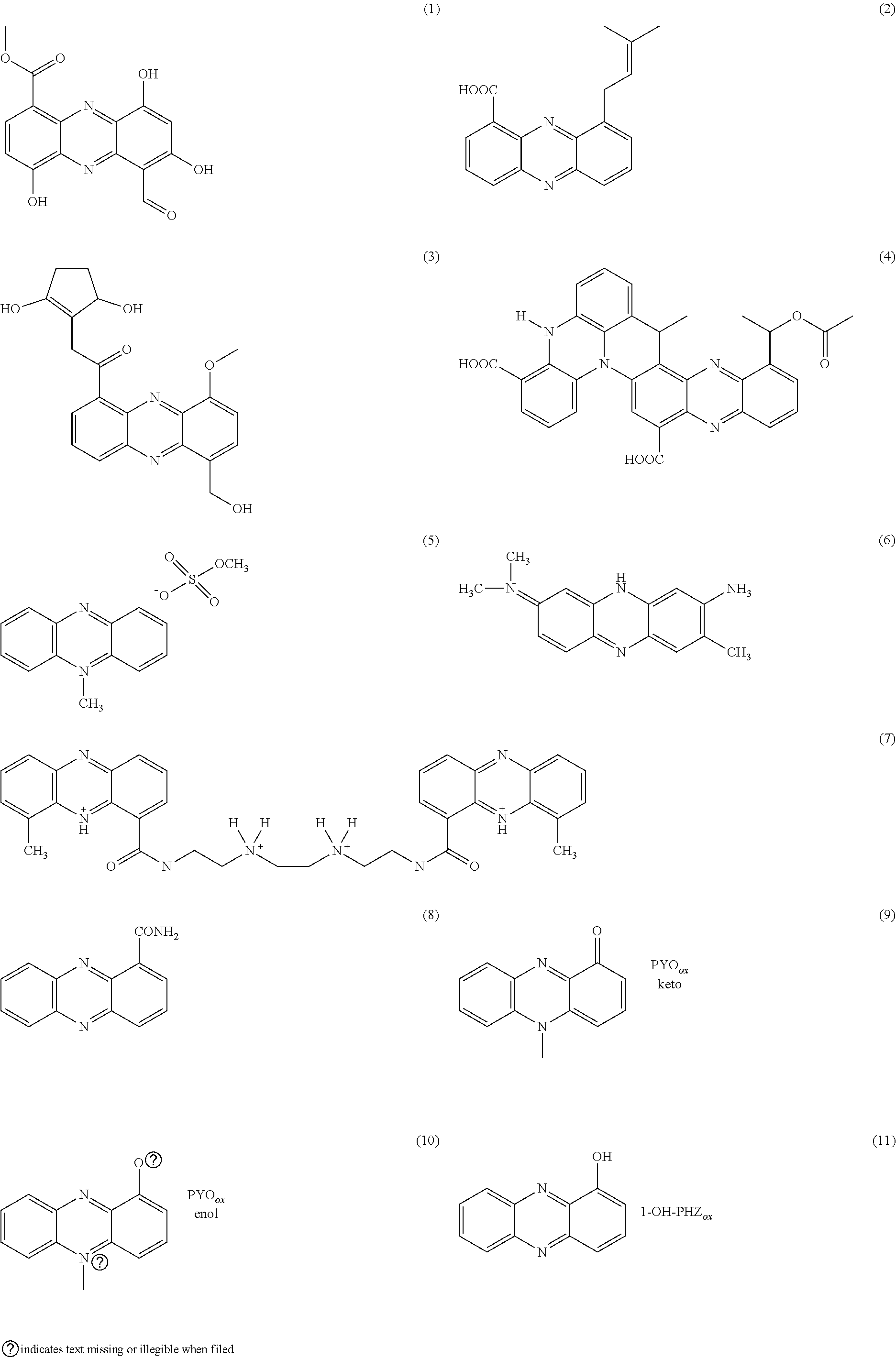

as well as additional phenazines that can be identified by a skilled person such as the exemplary phenazines described in Mentel et al. [7] and Pierson et al. [8] and in other references cited in the instant disclosure of each which is incorporated herein by reference in their entirety.

Phenazine pigments are mostly water soluble and are excreted into the medium. For example, pyocyanin produced by Pseudomonas aeruginosa, diffuses readily into agar-solidified media which become stained blue. Some phenazines are only sparingly water soluble and precipitate. For examples, chlororaphine, a mixture of phenazine-1-carboxamide (oxychlororaphine) and its dihydro derivative, produced by Pseudomonas chlororaphis, accumulate as isolated emerald-green crystals at the base of agar slants. Iodinine crystallizes on the surfaces of old colonies of Brevibacterium iodimum, giving them a dark-purple appearance, and phenazine-1-carboxylic acid (PCA) is deposited as golden yellow crystals in colonies of Pseudomonas aureofaciens and in the surrounding medium. It is noted, however, that the same pigment can be produced by unrelated bacteria and “achromogenic” strains of many phenazine-producers are common. A number of strains of bacteria produce more than one phenazine. It seems likely that all bacterial phenazines are derived from a common precursor.

Representative phenazines comprise pyocyanin (PYO) and Phenazine-1-carboxylic acid (PCA). Pyocyanin (PYO) is the phenazine characteristically produced by chromogenic strains of the pseudomonad, which is found as the blue pigment occasionally seen on infected wound dressings. More attention has been paid to pyocyanin than to any other phenazine. Pyocyanin is an organic base, blue in alkaline aqueous solutions but red when acidified. The differential solubility of these forms in chloroform and water was exploited for this pigment. Pyocyanin was found to be chemically reduced to a colorless form and spontaneously reoxidized in air, which has led to the discovery, the indicator and redox properties of the compound. Additionally, pyocyanin slowly decomposed to a yellow substance, no longer basic in nature, now known to be 1-hydroxyphenazine.

PCA is a yellow crystalline compound naturally produced by P. aureofaciens. The phenazine produced was readily extracted from acidified cultures with chloroform. Dilute alkali changed the color of the phenazine to orange-red and rendered it insoluble in chloroform. PCA isolated from cultures, in amounts of up to 1 g of pigment litre⁻¹, was shown to have antibacterial activity towards a number of plant pathogens.

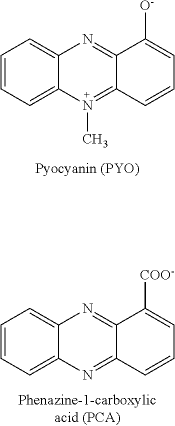

Biosynthesis as well as properties of individual phenazines are identifiable by a skilled person. In particular, phenazine natural products have been implicated in the virulence and competitive fitness of producing organisms. For example, the phenazine pyocyanin produced by Pseudomonas aeruginosa contributes to its ability to colonize the lungs of cystic fibrosis (CF) patients. Production of pyocyanin by P. aeruginosa is responsible for the bluish tint of sputum and pus associated with P. aeruginosa infections in humans. Clear correlation has been demonstrated between phenazine concentration in sputum and lung function decline. Further, phenazines are found to affect bacterial community development for P. aeruginosa.

Similarly, phenazine-1-carboxylic acid, produced by a number of Pseudomonas spp., increases survival in soil environments and has been shown to be essential for the biological control activity of certain strains. Examples are provided below for two types of phenazines known as pyocyanin and phenazine-1-carboxylic acid, respectively. For more examples of the occurrence, biochemistry and physiology of phenazine production, see Turner et al., [9].

Phenazines targeted by methods and systems and related compositions herein described, comprise in particular pyocyanin-like phenazines which are formed by phenazines of formula (IV)

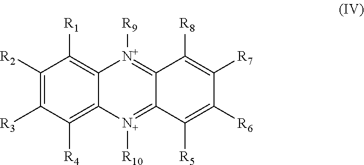

wherein R1-R10 are independently selected from hydrogen, hydroxy, alkoxy, alkyl, alkenyl, alkynyl, aryl, heteroaryl, acyl, and one of R1-R10 is a negatively charged substituent. In some of those embodiments, R₁—R₈ are independently selected from hydrogen, hydroxy, C1-C4 alkoxy, C1-C4 alkyl, C2-C4 alkenyl, C2-C4 alkynyl, and other groups identifiable to the skilled person, N+—R10 is H and R9 is CH3.

In some embodiments, pyocyanin-like phenazines comprise phenazines of formula III wherein at least one of R₁—R₈ is a hydroxy group. In some embodiments, pyocyanin-like phenazines comprise phenazines of formula (IV) wherein at least one of R₁—R₈ is methoxy group.

In some embodiments, pyocyanin and/or a pyocyanin-like phenazines can be represented by formula (V)

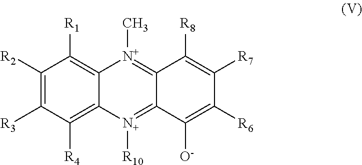

where R₁—R₄, R₆—R₈ and R₁₀ are independently selected from hydrogen, hydroxy, alkoxy, alkyl, alkenyl, alkynyl, aryl, heteroaryl, acyl, and other groups identifiable to the skilled person.

In some embodiments, pyocyanin-like phenazines comprise -methylphenazinium methyl sulfate or 3,6-diamino-10-methylacridinium or 1,8-dimethoxyphenazine or myxin, 5-methylphenazinium methyl sulfate or 3,6-diamino-10-methylacridinium or 1,8-dimethoxyphenazine or myxin and additional phenazines identifiable by a skilled person.

Pyocyanin, phenazines and other redox active compounds are produced by redox active bacteria.

The term “bacterium” or “bacteria” as used herein refers to a prokaryotic microbial species of Gram-negative or Gram-positive bacteria. The wording “Gram-negative bacteria” refers to bacteria that do not retain crystal violet dye in the Gram staining protocol. In contrast, the wording “Gram-positive bacteria” refers to those that are stained dark blue or violet by Gram staining.

The term “bacteria” or “bacterial cell”, as used herein indicates a large domain of prokaryotic microorganisms. Typically a few micrometers in length (from 0.5 to 6 um), bacterial cell can have a diameter from 1 to 10 μm or be as large as 750 um as will be understood by a skilled person. Bacteria in the sense of the disclosure refers to several prokaryotic microbial species which comprise Gram-negative bacteria, Gram-positive bacteria, Proteobacteria, Cyanobacteria, Spirochetes and related species, Planctomyces, Bacteroides, Flavobacteria, Chlamydia, Green sulfur bacteria, Green non-sulfur bacteria including anaerobic phototrophs, Radioresistant micrococci and related species, Thermotoga and Thermosipho thermophiles as would be understood by a skilled person. Taxonomic names of bacteria that have been accepted as valid by the International Committee of Systematic Bacteriology are published in the “Approved Lists of Bacterial Names” [10] as well as in issues of the International Journal of Systematic and Evolutionary Microbiology. More specifically, the wording “Gram positive bacteria” refers to cocci, nonsporulating rods and sporulating rods that stain positive on Gram stain, such as, for example, Actinomyces, Bacillus, Clostridium, Corynebacterium, Cutibacterium (previously Propionibacterium), Erysipelothrix, Lactobacillus, Listeria, Mycobacterium, Nocardia, Staphylococcus, Streptococcus, Enterococcus, Peptostreptococcus, and Streptomyces. Bacteria in the sense of the disclosure refers also to the species within the genera Clostridium, Sarcina, Lachnospira, Peptostreptococcus, Peptoniphilus, Helcococcus, Eubacterium, Peptococcus, Acidaminococcus, Veillonella, Mycoplasma, Ureaplasma, Erysipelothrix, Holdemania, Bacillus, Amphibacillus, Exiguobacterium, Gracilibacillus, Halobacillus, Saccharococcus, Salibacillus, Virgibacillus, Planococcus, Kurthia, Caryophanon, Listeria, Brochothrix, Staphylococcus, Gemella, Macrococcus, Salinococcus, Sporolactobacillus, Marinococcus, Paenbacillus, Aneurinibacillus, Brevibacillus, Alicyclobacillus, Lactobacillus, Pediococus, Aerococcus, Abiotrophia, Dolosicoccus, Eremococcus, Facklamia, Globicatella, Ignavigranum, Carnobacterium, Alloiococcus, Dolosigranulum, Enterococcus, Melissococcus, Tetragenococcus, Vagococcus, Leuconostoc, Oenococcus, Weissella, Streptococcus, Lactococcus, Actinomyces, Arachnia, Actinobaculum, Arcanobacterium, Mobiluncus, Micrococcus, Arthrobacter, Kocuria, Nesterenkonia, Rothia, Stomatococcus, Brevibacterium, Cellulomonas, Oerskovia, Dermabacier, Brachybacterium, Dermatophilus, Dermacoccus, Kytococcus, Sanguibacter, Jonesia, Microbacteirum, Agrococcus, Agromyces, Aureobacterium, Cryobacteriur, Corynebacteriun, Dietzia, Gordonia, Skermania, Mycobacterium, Nocardia, Rhodococcus, Tmukamurella, Micromonospora, Propioniferax, Nocardioides, Streptomyces, Nocardiopsis, Thermomonospora, Actinomadura, Bifidobacterium, Gardnerella, Turicella, Chlamydia, Chlamydophila, Borrelia, Treponema, Serpulina, Leptospira, Bacteroides, Porphyromonas, Prevotella, Flavobacterium, Elizabethkingia, Bergeyella, Capnocytophaga, Chryseobacterium, Weeksella, Myroides, Tannerella, Sphingobacterium, Flexibacter, Fusobacterium, Streptobacillus, Wolbachia, Bradyrhizobium, Tropheryma, Megasphera, Anaeroglobus, Escherichia-Shigella, Kiebsiella, muribaculum, alloprevotella, Paraprevotella, Oscillibacter, candidatus arthromitus, Aeromonas, romboutsia, Campylobacter, Salmonella, Faecalibacterium, Roseburia, Blautia, orihacterium, Ruminococcus.

Bacteria have a number of shapes, ranging from spheres to rods and spirals, and are present in most habitats on Earth, such as terrestrial habitats like deserts, tundra, Arctic and Antarctic deserts, forests, savannah, chaparral, shrublands, grasslands, mountains, plains, caves, islands, and the soil, detritus, and sediments present in said terrestrial habitats; freshwater habitats such as streams, springs, rivers, lakes, ponds, ephemeral pools, marshes, salt marshes, bogs, peat bogs, underground rivers and lakes, geothermal hot springs, sub-glacial lakes, and wetlands; marine habitats such as ocean water, marine detritus and sediments, flotsam and insoluble particles, geothermal vents and reefs; man-made habitats such as sites of human habitation, human dwellings, man-made buildings and parts of human-made structures, plumbing systems, sewage systems, water towers, cooling towers, cooling systems, air-conditioning systems, water systems, farms, agricultural fields, ranchlands, livestock feedlots, hospitals, outpatient clinics, health-care facilities, operating rooms, hospital equipment, long-term care facilities, nursing homes, hospice care, clinical laboratories, research laboratories, waste, landfills, radioactive waste; and the deep portions of Earth's crust, as well as in symbiotic and parasitic relationships with plants, animals, fungi, algae, humans, livestock, and other macroscopic life forms.

Redox active bacteria comprise proteobacteria such as Pseudomonales Burkholderiales Xanihomonadales, Burkholderiales and Enierobacteriales, or actinobacteria such as Streptomycetales, Pseudonocardiales. Micromonosporales, Streptosporangiales Corynebacleriales, Micrococcales, and others described for example in Dar et al 2020 [11]

Accordingly, exemplary redox active bacteria comprise one or more bacteria of the order Pseudomonales Burkholderiales Xanthomonadales, Burkholderiales and Enterobacteriales, Streptomycetales, Pseudonocardiales, Micromonosporales, Streptosporangiales Corynebacteriales, and/or Micrococcales.

Exemplary redox-active bacteria also comprise bacteria of the genera Streptomyces, Pseudomonas Staphylococcus, Klebsiella Enterobacter, Escherichia Brevibacterium and Mycobacteria. In particular redox active bacteria comprise bacteria of the genera Streptomyces and Pseudomonas, including P. aeruginosa, P. oryzihabitans, and P. luteola, Staphylococcus, Klebsiella, Enterobacter and Escherichia.

Exemplary bacteria in the sense of the disclosure comprise Pseudomonas, Brevibacterium, Coryneform Bacteria, Nocardia Brevibacterium linens, Brevibacterium, Burkholderia cenocepecia, Methanosarcina mazei, Mycobacterium abscessus. Pantoea agglomerans, Pectobacterium atrosepticum, Pelagio variabilis, Pseudomonas fluorescens, Streptomyces anulatus, Streptomyces cinnamonensis, Shewanella onidensis and related species that produce phenazines to facilitate various physiological functions identifiable to a skilled person upon reading of the present disclosure.

In some embodiments redox active bacteria comprise Staphylococcus aureus, Klebsiella pneumoniae, Enterobacter spp. and Escherichia coli as will be understood by a skilled person.

In some embodiments, herein described, redox active bacteria in the sense of the disclosure comprise phenazine producing bacteria, which comprise Pseudomonas aeruginosa and additional bacteria known or identifiable by a skilled person, as opposed to phenazine degrading bacteria which comprise Sghingomonas sp. DP58 (see Yang et al. [12] and additional bacteria known or identifiable by a skilled person.

An overview of the exemplary phenazines and phenazines producing bacteria producing them are reported in the following Table 3 adapted from Turner and Messenger (1986). [9]

TABLE 3
Exemplary Substituent patterns for naturally-occurring phenazines and their bacterial
sources. Where a substituent position is blank in the table, the substituent = H.

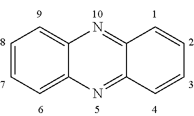 [SMILES: c1ccc2nc3ccccc3nc2c1]

| Phen- | | | | | | | | | | | Bacterial |
|---|---|---|---|---|---|---|---|---|---|---|---|
| azine | 1 | 2 | 3 | 4 | 5 | 6 | 7 | 8 | 9 | 10 | source |
| VI | OH | | | | | | | | | | 3, 20 |
| VII | OCH₃ | | | | | | | | | | 22 |
| VIII | OH | | | | | | | | | O | 16 |
| IX | OH | | | | CH₃ | | | | | | 1, 18 |
| X | OH | | | | | | | NH₂ | | | 6 |
| XI | | OH | | | | | | | | | 3 |
| XII | OH | | | | | OH | | | | | 5, 7, 8, 9, |
| | | | | | | | | | | | 11, 12, |
| | | | | | | | | | | | 15, 16, |
| | | | | | | | | | | | 20, 29 |
| XIII | OH | | | | | OCH₃ | | | | | 20 |
| XIV | OCH₃ | | | | | OCH₃ | | | | | 20, 22 |
| XV | OH | | | | O | OH | | | | | 5, 7-16, |
| | | | | | | | | | | | 20, 29 |
| XVI | OH | | | | O | OH | | | | O | 5, 7-16, |
| | | | | | | | | | | | 20, 29 |
| XVII | OH | | | | O | OCH₃ | | | | O | 32 |
| XVIII | OH | | | | | | | OH | | | 5, 6 |
| XIX | OH | | | | | | | OH | | O | 5, 6 |
| XX | | OH | OH | | | | | | | | 3 |
| XXI | | OH | OH | | | | OH | | | | 3 |
| XXII | OCH₃ | OCH₃ | | | | OCH₃ | | | | | 22 |
| XXIII | COOH | | | | | | | | | | 1, 2, 3, 5, |
| | | | | | | | | | | | 16, 19, |
| | | | | | | | | | | | 26 |
| XXIV | C(O)OCH₃ | | | | | | | | | | 22 |
| XXV | C(O)NH₂ | | | | | | | | | | 1, 2 |
| XXVI | COOH | | | | CH₃ | | NH₂ | | | | 1 |
| XXVII | COOH | | SO₃H | | CH₃ | | NH₂ | | | | 1 |
| XXVIII | COOH | OH | | | | | | | | | 3 |
| XXIX | COOH | | | OH | | | | | | | 3 |
| XXX | COOH | | | | | OH | | | | | 3, 5 |
| XXXI | C(O)OCH₃ | | | | | OCH₃ | | | | | 22 |
| XXXII | COOH | | | | | | | | OH | | 5, 6 |
| XXXIII | COOH | OH | OH | | | | | | | | 3 |
| XXXIV | COOH | OH | | | | OH | | | | | 3 |
| XXXV | COOH | OH | | | | | | | OH | | 5, 6 |
| XXXVI | COOH | OH | OH | OH | | | | | | | 3 |
| XXXVII | COOH | | | | | COOH | | | | | 3, 4, 5, 6 |
| XXXVIII | COOH | | | | | CH₂OH | | | | | 17 |
| XXXIX | C(O)OCH₃ | | | | | CH(OH)CH₃ | | | | | 28 |
| XL | C(O)OCH₃ | | | | | CH(OCH₃)CH₃ | | | | | 28 |
| XLI | COOH | | | | | CH₂CHC(CH₃)₂ | | | | | 26 |
| XLII | COOH | | | | | CH(CH₃)— | | | | | 25, 27 |
| | | | | | | OC(O)C₇H₇O | | | | | |
| XLIII | [C(O)OCH₃ | | | | | CHCH₃]₂ | | | | | 28 |
| XLIV | COOH | OH | | | | COOH | | | | | 3 |

1 = Pseudomonas aeruginosa;
2 = P. chlororaphis;
3 = P. aureofaciens;
4 = P. cepacia;
5 = P. phenazinium;
6 = unidentified non-motile Gram-negative rod;
7 = Brevibacterium iodinum;
8 = B. crystalloiodinum;
9 = B. stationis var. iodinofaciens;
10 = B. maris;
11 = Corynebacterium hydrocarboclastum;
12 = Arthrobacter paraffineus;
13 = Micrococcus paraffinolyticus;
14 = Streptosporangim amethystogenes var. nonreducens;
15 = Nocardia hydrocarbonoxydans;
16 = Actinomadura (=Nocardiopsis) dassonvillei:
17 = Streptomyces griseoluteus;
18 = S. cyanoflavus;
19 = S. misakiensis;
20 = S. thioluteus;
21 = S. lomondensis;
22 = S. luteoreticuli;
23 = S. recifensis;
24 = S. endus sub sp. Aureus;
25 = S. canarius;
26 = S. cinnamonensis;
27 = Strepromyces strain NRRL 12067;
28 = Streptomyces strain ME 679-m4;
29 = Microbispora aerata;
30 = M . amethystogenes;
31 = M. parva;
32 = Sorangium sp.

Phenazines and other redox active compound in the sense of the disclosure can be further characterized by various electrical properties such as a standard electrode potential E° vs. NHE of −500 mV to 500 mV and other properties such as number of redox cycle over a certain period of time (e.g. number of days) as ability to support survival and susceptibility to reduction by enzymes and/or bacterial strains.

1 Exemplary redox-active compounds with corresponding exemplary properties listed in Table 4.

TABLE 4
Exemplary redox-active compounds

| | | | # of | | |
|---|---|---|---|---|---|
| | | E⁰, | Redox | | |
| Chemical | | (vs. | cycles | | |
| name | Structure | NHE) | over 7 | Support | Reduction |
| (Abbreviation) | (The oxidized form) | (mV) | days | survival? | by PA14? |
| Pyocyanin (PYO) | 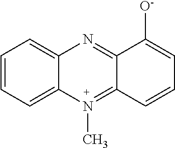 | −40^{a} | 31 | Yes | Yes |
| Phenazine-1- carboxylate (PCA) | 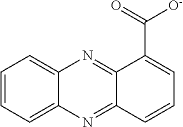 [SMILES: O=C([O-])c1cccc2nc3ccccc3nc12] | −114^{a} | 22 | Yes | Yes |
| 1- Hydroxy- phenazine (1-OHPHZ) | 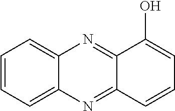 [SMILES: Oc1cccc2nc3ccccc3nc12] | −174^{a} | 14 | Yes | Yes |
| Methylene blue (MB) | 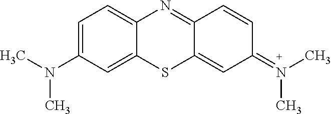 | 0^{b} (+11^{c}) | 3 | No | Yes |
| 2,6-AQDS | 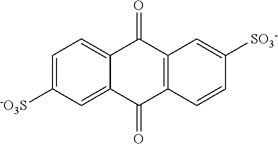 | −184^{d} | No cycle | No | Yes (very slowly) |
| Paraquat | 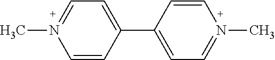 | −446^{c} | No cycle | No | No |
| Homogentisic acid (HMA) | 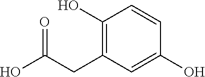 [SMILES: O=C(O)Cc1cc(O)ccc1O] | +306^{b} | No cycle | No | — |

^{a}Reference [13] [14]
^{b}E⁰, values were measured in aqueous solution at pH 7 in this study
^{c}Reference (Fultz, M. L., and R. A. Durst. 1982. Mediator Compounds for the Electrochemical Study of Biological Redox Systems—a Compilation. Analytica Chimica Acta 140:1-18) [15]
^{d}Reference [16] [17]
^{e}References [18, 19] [20]

A skilled person can identify the properties of additional phenazines and/or redox active compounds in the sense of the disclosure upon reading of the present disclosure.

Identification of redox active bacteria and related redox active compounds can be performed by various techniques identifiable by a skilled person. specific absorption and fluorescence that can that can be diagnostic, as well as mass spectrometry, liquid chromatography+mass spectrometry or UV/VIS absorption, electrochemistry, and spectral methods which can be diagnostic (see e.g. [21][22]).

Additionally redox compounds such as phenazines can be extracted from bacterial cultures using organic solvents as will be understood by a skilled person[21][22].

The term “medium” as used herein indicates an environment that is suitable to support growth, life and or survival of microorganisms or cells. An “environment” as used herein indicates the complex of physical, chemical, and biotic factors (such as climate, soil, and living things) that act upon a microorganism, cell or organism. Environments comprise individuals, other organisms or portions thereof (e.g. organs, tissues or cells) as well as physical objects.

The term “individual” or “host” as used herein indicates any multicellular organism that can comprise microorganisms, thus providing a biological environment for microbes and in in particular an environment for microbial communities, in any of their tissues, organs, and/or biofluids. Exemplary individual in the sense of the disclosure includes plants, algae, animals, and in particular, vertebrates, mammals more particularly humans. Exemplary tissues organs and/or biofluids from an individual comprise the following: whole venous and arterial blood, capillary blood, blood plasma, blood serum, dried blood spots, cerebrospinal fluid, interstitial fluid, sweat, lumbar fluid, nasal tissues and fluids, sinus tissues and fluids, tears, corneal, saliva, sputum or expectorate, bronchoscopy secretions, transtracheal tissue and/or fluid, endotracheal tissue and/or fluid, bronchoalveolar tissue and/or fluid, gastric tissue and/or fluid, colon tissue and/or fluid, subcutaneous and mesenteric adipose tissue and/or fluid, bile, vaginal tissue and/or fluid such as secretions, endometrial tissues and/or fluids such as secretions, urethral fluids and secretions, mucosal secretions, synovial fluid, ascitic fluid, peritoneal tissue and/or fluid, tympanic membrane fluid, urine, including clean-catch midstream urine, catheterized urine, suprapubic tissue and fluids, kidney stones, prostatic secretions, feces, mucus, pus, wound, skin, hair, nail, cheek tissue, bones, bone marrow, muscular tissues solid organ, solid organ tissue such as lung tissues, breast milk, or tumor cells, among others identifiable by a skilled person. The medium can be in vivo as part of the individual or in vitro or ex vivo as part of sample taken from an individual will also be understood by a skilled person.

Suitable medium are environments such growth medium or culture medium in a liquid or gel designed to support the bacteria in vitro, as well as tissues and other suitable environments within a host individual (including a human host) in vivo. Accordingly, various mediums are formed by or comprise medium components that are chemical compounds and molecules that are used in life-supporting functions and processes of bacteria, which allow bacterial cells to grow and reproduce.

Exemplary medium components comprise at least one redox-active compound in a solvent. In some embodiments, the solvent can comprise water in at least 10% by volume, preferably at least 50% by volume, and most preferably at least 95% by volume.

An exemplary medium is therefore typically aqueous medium at pH 6.5-8.0 electrolyte salts such as potassium sulfate, magnesium chloride, protein hydrolysates <500 mM, 0-20% salinity and is at a temperature of 8-37° C. compatible with the physiology of the bacteria.

In some embodiments, the medium solvent can comprise at least one organic solvent such as ethanol, methanol, tetrahydrofuran, dimethylformamide (DMF), dimethyl sulfoxide (DMSO), acetic acid, formic acid, glycerol, glycol, isopropanol and 1-butanol. Additional medium components that can be found in a medium comprise amino acids, salts, polyacrylic acids, polyols, polyglycols, such as Polyethylene Glycols (e.g. PEG 1000, PEG 3000), polysaccharides, polypeptides, polynucleotides as well as other organic polymers with molecular weight between 10,000 to 1,000,000 Da and additional components identifiable by a skilled person. For example medium components can comprise sodium thioglycolate (HS—CH₂CO₂Na), sodium dithionite, Organic: simple sugars e.g. glucose, acetate or pyruvate; extracts such as peptone, tryptone, yeast extract etc., hydrogen carbonate salts (HCO₃⁻), amino acids, NH₄Cl, (NH₄)₂SO₄, KNO₃, KCI, K₂HPO₄, MgCl₂, MgSO₄, CaCl₂, Ca(HCO₃)₂, FeCl₃, Fe(NH₄)(SO₄)₂, Fe-chelates, CoCI₂, ZnCl₂, Na₂MoO₄, CuCl₂, MnSO₄, NiCl₂, Na₂SeO₄, Na₂WO₄, Na₂VO₄, Vitamins, amino acids, purines, pyrimidines and additional components identifiable by a skilled person. (see e.g. [23] for Pseudomonas aeruginosa cultivation).

Exemplary media in the sense of the disclosure comprise physical objects such as tools and equipment including components capable of supporting life growth and/or survival of bacteria. Exemplary physical objects include medical devices implanted in the body such as catheters, osteoimplants or heart valves, contact lenses. protective clothing, respiratory equipment, tools, such as medical tools, laboratory equipment and ventilators which can comprise redox active bacteria on one or more parts and in particular one or more surface.

In some embodiments, redox active bacteria comprise persister cells which typically constitute a small portion of a culture which is tolerant to killing by lethal doses of bactericidal antibiotics. Persister bacterial cells can be identified, for example, by exposure of logarithmic or stationary cultures of the bacteria to antibiotics using concentrations exceeding five times the minimum inhibitory concentration for each antibiotic. Persister numbers can be determined by plating the antibiotic-treated cultures on LB agar plates and subsequent counting of colony forming units representing the cell numbers which survived antibiotic exposure. Other methods for identification of persister cells will be known by a skilled person, and can be found, for example, in Möker et al. [24]

In some embodiments, phenazine producing bacteria are further characterized by a phenazine-mediated bacterial biofilm development in the bacteria.

As used herein the term “biofilm” indicates an aggregate of microorganisms in which cells adhere to each other on a surface. These adherent cells are frequently embedded within a self-produced matrix of extracellular polymeric substance (EPS). Biofilms can form on living or non-living surfaces and can be prevalent in natural, industrial and hospital settings. The microbial cells growing in a biofilm are physiologically distinct from planktonic cells of the same organism, which, by contrast, are single-cells that can float or swim in a liquid medium. Formation of a biofilm begins with the attachment of free-floating microorganisms to a surface. These first colonists adhere to the surface initially through weak, reversible adhesion via van der Waals forces. If the colonists are not immediately separated from the surface, they can anchor themselves more permanently using cell adhesion structures such as pili. When the biofilm growth is balanced with that of biofilm dispersion, the biofilm is considered “mature.” Methods to quantify and measure biofilms will be known to a skilled person and can include, for example, the COMSTAT method of Heydorn et al. [25].

Formation of biofilms in phenazine producing bacteria and therefore conversion of the related cells from flee-floating to colonists of a biofilm formation is associated with a survival ability of the bacteria. Survival of cells within oxygen-limited regions in these biofilms is enabled by extracellular electron transfer (EET), whereby small redox active molecules act as electron shuttles to access distal oxidants.

Accordingly, biofilms provide bacterial cells with a protective environment where persistence and antibiotic tolerance arise, making them a leading contributor to chronic infections ([26]). Extracellular electron transfer (EET) pathways have been recurrently found among biofilm-forming opportunistic pathogens ([27][28][17]). Such pathways are often dependent on the redox cycling of either self-made or borrowed small molecules that serve as electron shuttles between cells in the biofilm and extracellular terminal electron acceptors ([29]). Specifically, in the biofilms formed by Pseudomonas aeruginosa PA14 ([30]), oxygen limitation within anoxic regions is overcome through the use of phenazines as electron shuttles to reduce distal oxygen ([31][32]). Of the different phenazines produced by P. aeruginosa PA14, pyocyanin (PYO) is present at high abundance and facilitates EET via its association with extracellular DNA in the biofilm matrix ([33]).

Accordingly, the methods and systems and related devices that are based on the surprising finding that it is possible to perform electrochemical control of the red-ox cycle of redox active compounds to inhibit and reduce viability up to killing the redox active bacteria that produce them.

The wording “electrochemical control’ as used herein with respect to bacteria indicates control over one or more aspects of bacteria physiology obtained through application of a difference in voltage to a medium possibly containing or known to contain the bacteria or onto an agent.

In particular, in methods and systems herein described and related composition and devices, a reductive electrical potential is applied to a medium known to contain and/or possibly including redo active bacteria to increase in the medium the amounts of the corresponding redox active compound which is in a reduced state.

More specifically methods and systems of the present disclosure are based on the surprising finding that applying a reducing potential to the medium containing the redo active bacteria it is possible to effectively disrupt the redox cycling of the corresponding redox active compound by maintaining the redox active compound in the reduced state. Maintaining redox active compound in a reduced state can results in a decrease viability up to kill cells even when included in a biofilm formation.

Accordingly, in embodiments of the present disclosure control of the redox states of redox active compound is performed through application of a negative electrical potential for a time and under condition to create a reductive environment in a medium containing bacteria thus reducing the phenazines produced by the bacteria and thereby inhibiting viability of the corresponding redox active bacteria.

Methods and systems of the disclosure thus leverage the red ox active cycle of the redox active compounds to obtain the desired effect.

Redox active compounds typically undergo redox active cycling of the YR5 and YR10 moieties, often via a coupled 2 electron, 2 proton transfer mechanism in polar protic solvent (e.g., water), with the reduction process shown below (left to right):

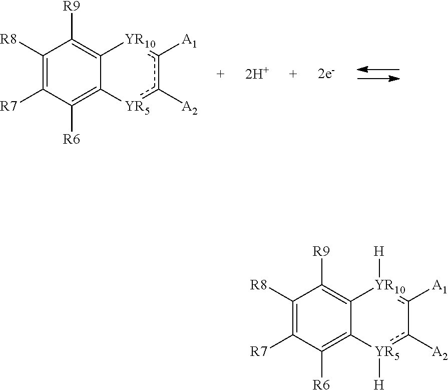

noting that the protons can be added to the R5 or Y atoms, and/or R10 or Y atoms, in particular when Y is N and R10 is null as will be understood by a skilled person upon reading of the disclosure.

In particular, redox active compounds where Y is carbon typically undergo redox cycle of the CO moieties, often via a coupled 2 electron, 2 proton transfer mechanism in polar protic solvent (e.g., water), with the reduction process shown below (left to right):

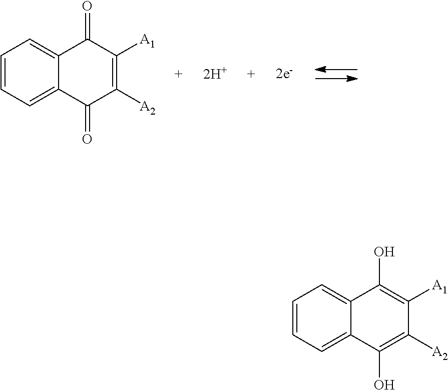

Redox active compounds where Y is N typically undergo redox cycles of the N moieties, often via a coupled 2 electron, 2 proton transfer mechanism in polar protic solvent (e.g., water), with the reduction process shown below (left to right):

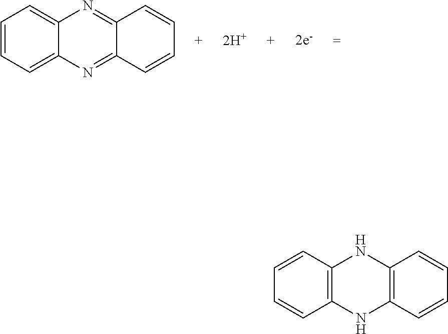 [SMILES: c1ccc2nc3ccccc3nc2c1 ; c1ccc2c(c1)Nc1ccccc1N2]

In the oxidized form, phenazines that are substituted at the N atoms (5- and/or 10-positions) may carry a positive charge. Similarly, substituents that may be protonated and/or deprotonated under biological pH conditions (e.g. NH₂, NHR, COOH, SO₃H and/or OH or SH conjugated into the phenazine ring) can be present in either protonated or deprotonated form.

Pyocyanin-like compounds of formula (IV) provide representative exemplars of bacterial redox active phenazines, with a positive charge on the substituted N-atom and a negatively-charged group in conjugation with this, substituted on the ring in particular in position 1 and in particular present as an O⁻ group. More particularly, pyocyanin-like compounds of formula (V) can have a substituent at the 1-position (as found for many of these phenazines), for example an OH group which is electron-donating; it is produced by at least two common strains of bacteria that also produce other characteristic phenazine structures.

Pyocyanin-like phenazines of formula (IV) and (V) also provide representatives of bacterial redox active molecules of Formula (I) because the redox properties of the pyocyanin for the N=C moieties would be understood by a skilled person to be representative of redox properties of YR5 and YR10 moieties of the redox compounds of Formula (I) inclusive of embodiments when YR5 and YR10 is C═O. In all cases, substituent groups may be selected such that the redox active molecules of Formula (I) undergo reduction processes at the midpoint potential ranges of interest described, and the redox-cycle mechanism is a coupled electron/proton transfer process.

Pyocyanin-like phenazines of formula (IV) and (V) also provide representatives of bacterial redox active molecules in general because the redox properties of the pyocyanin for the N=C moieties would be understood by a skilled person to be representative of redox properties of corresponding moieties in redox active compound which undergo reduction processes at the midpoint potential ranges of interest described, and the redox-cycle mechanism is a coupled electron/proton transfer process.

Pyocyanin phenazines and other redo active compounds herein described are characterized by a midpoint potential. The wording “midpoint potential”, “formal potential”, “half-wave potential” or “mid-peak potential” or “E½” as used herein indicates the reduction potential of a particular redox compound under a given condition (solvent, pH).

In particular, a “midpoint potential” of a redox active compound in the sense of the disclosure is the potential with respect to a reference electrode under certain environmental conditions where the activities of the redox active compound in the oxidized state and in the reduced state are equal.

E_{1/2} values of a given compound can be measured by a variety of experimental procedures as may be understood by the skilled person, including cyclic voltammetry (described above), differential pulse voltammetry, square wave voltammetry, direct current polarography and redox titration; suitable methods are described in Bard et al 2022 [34].

For example, making reference to the schematics of the preceding paragraph in a cyclic voltammetry experiment phenazines will show a peak on the cathodic sweep (denoted E_{pc}) corresponding to the reduction of the phenazine group (left to right):

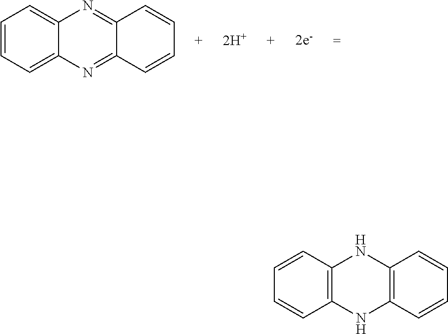 [SMILES: c1ccc2nc3ccccc3nc2c1 ; c1ccc2c(c1)Nc1ccccc1N2]

The reverse, anodic sweep with show a peak (denoted Eₚₐ) corresponding to the re-oxidation of the phenazine group (right to left). If the electrode reaction is diffusion-controlled and reversible, the cathodic and anodic peak currents will be equal in area, and the mid-point between these peaks may be taken as the formal reduction potential of the phenazine under the experimental conditions used. This value can interchangeably be referred to as the “midpoint potential” or “mid-peak potential”, denoted E_{1/2} as will be understood by a skilled person upon reading of the present disclosure (see also Examples 13 to 16 of the present disclosure).

E_{1/2} values are measured relative to a reference electrode. For aqueous systems, as for redox compounds produced by bacteria, suitable reference electrodes include the normal hydrogen electrode (NHE), saturated calomel electrode (SCE) and silver/silver chloride electrodes, among others. E_{1/2} values measured relative to one of these standard electrodes may be converted to values relative to other standard electrodes by simple addition or subtraction of the respective potential values, as can be found in [35] Most usefully, for reference, SCE has a potential +0.244 V vs NHE, and Ag/AgCl (3M KCl) has a potential +0.197 V vs. NHE as will be understood by a skilled person (see also Examples 13 to 16 of the present disclosure).

Unless otherwise indicated, the midpoint potential indicated in the present disclosure relates to midpoint potential in a medium at physiological conditions for the bacteria and thus an aqueous medium at pH 6.5-8.0 with electrolyte salts such as potassium sulfate, magnesium chloride, protein hydrolysates <500 mM, 0-20% salinity at a temperature of 8-37′° C.

A skilled person will be able to understand and identify any adjustments due to change in conditions operating within the limits of electrochemical parameter at a given pH which maintain the integrity of the water solvent herein-called “voltage window”, as the by-products of water electrolysis may impede wound healing or damage tissue as will be understood by a skilled person upon reading of the present disclosure (see also Examples 13 to 16 of the present disclosure).

Exemplary E_{1/2} values for bacterially-produced phenazines are given in Table below, taken from Wang and Newman (2008), incorporated by reference in its entirety [14]

| Structure | | E_{1/2} (vs. Ag/AgCl | | | |
|---|---|---|---|---|---|
| (from | Electrochemical reaction of bacterially- | (3M KCl))/mV | | | |
| Table 1) | produced phenazine | pH 5 | pH 6 | pH 7 | pH 8 |
| IX | 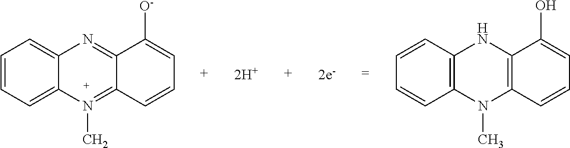 | −128 | −189 | −237 | −300 |
| XXIII | 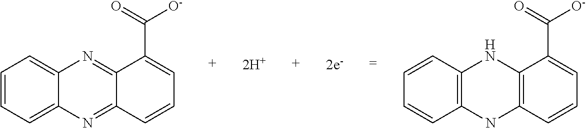 [SMILES: O=C([O-])c1cccc2nc3ccccc3nc12 ; O=C([O-])c1cccc2c1Nc1ccccc1N2] | −185 | −257 | −313 | |
| XXV | 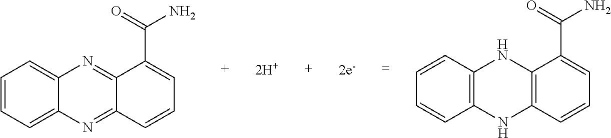 [SMILES: NC(=O)c1cccc2nc3ccccc3nc12 ; NC(=O)c1cccc2c1Nc1ccccc1N2] | −205 | −278 | −337 | |
| VI | 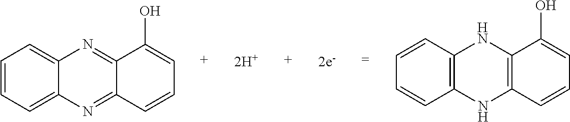 [SMILES: Oc1cccc2nc3ccccc3nc12 ; Oc1cccc2c1Nc1ccccc1N2] | −254 | −323 | −371 | −442 |

The redox properties of pyocyanin like phenazines (IV) and (V) are representative of these bacterial phenazines as they display a 2-electron 2-H coupled reduction reaction that is reversible under a range of conditions, with a standard potential that is <−100 mV vs. Ag/AgCl.

The present disclosure is based on the surprising finding that applying a negative voltage to a working electrode and counter electrode relative to a reference electrode which is selected to have a reducing potential in the working electrode lower than midpoint potential value of a target redox compound i) increases the electrochemical conversion of the compound into a reduced state and ii) interferes with the related redox cycle physiological to the corresponding redox active bacteria.

The electrochemical interference and blockage of the redox active cycle for a target redox active compound resulting from the reducing potential to the working electrode thus inhibit the redox cycle of the redox active compound which in turn can significantly inhibits the viability and/or survivability of the target bacteria. Reference is made to the data reported in the examples section showing that the related process can result in a reduction of colony forming units (CFU) in the medium up to 10-fold and even up to 100-fold as will be understood by a skilled person upon reading of the present disclosure (see Examples 1-4).

Accordingly, in methods and systems of the present disclosure a reducing potential is applied to the working electrode that is determined as a function of the E_{1/2} value of one or more redox active compounds produced by one or more redox active bacteria to inhibit viability and/or survivability of the bacteria up to killing and even eradicating the bacteria from the medium.

In particular, in methods and systems herein described a negative voltage is applied to a couple of working electrode and counter electrode relative to a reference electrode, such that the working electrode has a reducing potential lower than the Et₂ of the target redox compound produced by the target redox active bacteria. Accordingly, when the negative voltage is applied to the medium and the working electrode potential is lower than the midpoint potential of the target redox active compound, the target redox active compound is increasingly electrochemically converted from an oxidized state to a reducing state and held at a potential at which the target compound cannot be re-oxidized.

Accordingly in embodiments of the present disclosure, application of the negative voltage to the working and counter electrodes relative to a reference electrode, interferes with the redox cycle of the target redox active compound also interfering with the viability of the redox active bacteria which depend on said redox active cycle in one or more physiological processes.

In embodiments where the target redox compound is a phenazine and the target redox active bacteria is a phenazine producing bacteria, the reducing voltage difference can also be indicated as phenazine reducing voltage and the reducing potential of the working electrode can also be indicated as phenazine reducing potential.

In embodiments of the present disclosure the voltage source, the working electrode, the control electrode and the reference electrode can be used in many different configuration depending on the type of target medium and the shape and dimension of the target area of the medium as will be understood by a skilled person (see also Examples 5 to 12 of the present disclosure).

In particular, in preferred embodiments, the voltage source a working electrode a counter electrode and a reference electrode configured to present the working electrode for contact with a target area of the medium which is known or expected to comprise or possibly comprise the target redox bacteria (see e.g. Examples 5 to 12 of the present disclosure).

In particular in most preferred embodiments, the negative difference in potential is applied to the medium, under anaerobic conditions which maximize the inhibitory effect of the negative voltage application as will be understood by a skilled person upon reading of the present disclosure (see Examples 1-4).

The terms “inhibit” and “inhibition” as used herein refers to a decrease relative to a baseline level. Accordingly, inhibition of a viability and cell survival indicates a decrease in a related parameter compared to value of the parameter selected as a baseline level. Inhibition of the viability and/or survivability to a challenge. In particular, viability and/or survivability can be detected by detecting any products or other indicator and/or parameter associated with viability and/or survivability identifiable by a skilled person.

The term “viability” as used here in refers to whether or not a bacterial cell is able to maintain itself or recover its potentiality. Viable cells in the sense of the present disclosure are cells able to, or capable of recover the ability to form colonies and biofilms on or in a solid or liquid medium.

The term “survivability” as used herein with respect to cells indicates viability of a cells measured following an event interfering with the physiology of the cells, such as alteration of culture conditions, for example through modifications of pH, temperatures and/or slats concentrations, and/or administration of an antimicrobial or additional agents interfering with the physiology of the cells, such as application energy source (such as UV light) and/or of a voltage source to provide a reductive electrical potential to the medium.

Accordingly, in some embodiments a method is described to decrease viability of redox active bacteria in a medium, the redox active bacteria producing a redox active compound having an oxidized state and a reduced state, the redox active compound further having a midpoint potential, the method comprising:
contacting the medium with i) a working electrode having a working electrode potential compared to a reference electrode and ii) a counter electrode having a counter electrode potential compared to the reference electrode;
operating a voltage source to apply to the working electrode and the counter electrode a reducing voltage selected so that the working electrode potential has a reducing potential lower than the midpoint potential of the redox active compound, the operating performed for a time and under conditions to increase the concentrations of the redox active compound in the reduced state in the medium thus inhibiting viability of the redox active bacteria.

In some embodiments the method further comprises selecting a voltage between the working electrode and counter electrode based on the midpoint potential of the redox active compound so that the working electrode potential is lower than the midpoint potential of the redox active compound.

In particular, the selecting a voltage is performed to select the voltage that results in a working electrode potential lower than the midpoint potential within a water voltage selected to maintain the integrity of the medium. The “water voltage” being a voltage level where the integrity of the medium begins to be compromised.

In preferred embodiments, the reducing potential of the working electrode is from 50 mV lower than the E_{1/2} potential to 250 mV lower than the E_{1/2} of the target redox compound to drive the reaction. For example, if the E_{1/2} of a target compound is X mV, a preferred reducing potential of the working electrode can be (X-50 mV) to (X-250 mV).

In many embodiments the reducing potential of the working electrode is lower than E_{1/2} of the target redox active compound +/−20 mV. In some of these embodiments the redox active compound is a phenazine and in particular a pyocyanin like phenazine.

In some embodiments, E_{1/2} of one or more target redox active compounds is between +138 mV to −517 mV vs Ag/AgCl at pH 7, and the reducing potential of the working electrode can range from −50 mV to −600 mV, preferably from −100 mV to −550 mV at pH 7.

In some embodiments, E_{1/2} of one or more target redox active compounds is between 0 mV to −500 mV vs Ag/AgCl at pH 7, and the reducing potential of the working electrode can range from −50 mV to −600 mV, preferably from −100 mV to −550 mV at pH 7.

In some embodiments, E_{1/2} of one or more target redox active compounds is from 0 mV to −400 mV vs Ag/AgCl at pH 7, and the reducing potential of the working electrode can range from −50 mV to −600 mV, preferably from −100 mV to −500 mV at pH 7.

In some embodiments, E_{1/2} of one or more target redox active compounds is from 0 mV to −300 mV vs Ag/AgCl at pH 7, and the reducing potential of the working electrode can range from −50 mV to −550 mV, preferably from −100 mV to −450 mV at pH 7.

In some embodiments, E_{1/2} of one or more target redox active compounds is from 0 mV to −200 mV vs Ag/AgCl at pH 7, and the reducing potential of the working electrode can range from −50 mV to −550 mV, preferably from −100 mV to −500 mV at a pH 7.

In some embodiments, E_{1/2} of one or more target redox active compounds is from 0 mV to −300 mV vs Ag/AgCl at pH 7, and the reducing potential of the working electrode can range from −50 mV to −500 mV, preferably from −100 mV to −450 mV at a pH 7.

In some embodiments, E_{1/2} of one or more target redox active compounds is from 0 mV to −250 mV vs Ag/AgCl at pH7, and the reducing potential of the working electrode can range from −50 mV to −450 mV, preferably from −100 mV to −400 mV at a pH 7.

In some embodiments, E_{1/2} of one or more target redox active compounds is from 0 mV to −350 mV vs Ag/AgCl at pH7, and the bacteria reducing voltage difference applied can range from −50 mV to −600 mV, preferably from −100 mV to −550 mV at a pH 7.

In some embodiments, E_{1/2} of one or more target redox active compounds is from −100 to −500 mV vs Ag/AgCl at pH7, and the bacteria reducing voltage difference applied can range from −150 mV to −600 mV, preferably from −200 mV to −550 mV at a pH 7.

In some embodiments, E_{1/2} of one or more target redox active compounds is from −100 mV to −400 mV vs Ag/AgCl at pH7, and the reducing potential of the working electrode can range from −150 mV to −600 mV, preferably from −200 mV to −550 mV at a pH 7.

In some embodiments, E_{1/2} of one or more target redox active compounds is from −100 mV to −400 mV vs Ag/AgCl at pH7, and the reducing potential of the working electrode can range from −150 mV to −600 mV, preferably from −200 mV to −600 mV at a pH 7.

In some embodiments, E_{1/2} of one or more target redox active compounds is from −125 mV to −375 mV vs Ag/AgCl at pH7, and the reducing potential of the working electrode can range from −175 mV to −600 mV, preferably from −225 mV to −500 mV at a pH 7.

In some embodiments, E_{1/2} of one or more target redox active compounds is from 0 mV to −375 mV vs Ag/AgCl at pH7, and the reducing potential of the working electrode can range from −50 mV to −600 mV, preferably from −100 mV to −550 mV at a pH 7.

In embodiments herein described the reducing potential of the working electrode is applied for different times depending on the i) known or expected concentrations of target bacteria known or suspected to be present; ii) the known or expected concentrations of corresponding target redox-active compounds; iii) the type and electrochemical properties of the medium; iv) the presence or absence of simultaneous combined or sequential administration of antibiotics or other antimicrobial and v) the extent of inhibition of the viability of the target bacteria desired.

In some embodiments the reducing potential of the working electrode can be applied to a target area for a time ranging from 1 minute to 72 hours or more as will be understood by a skilled person.

In some embodiments the reducing potential of the working electrode can be applied to a target area for six hours, or up to 36 hours or up to 72 hours.

In some embodiments the reducing potential of the working electrode can be applied to a target area for 1-6 hours.

In some embodiments the reducing potential of the working electrode can be applied to a target area for 6-12 hours.

In some embodiments the reducing potential of the working electrode can be applied to a target area for 6-24 hours.

In some embodiments the reducing potential of the working electrode can be applied to a target area for 24-36 hours.

In some embodiments the reducing potential of the working electrode can be applied to a target area for 36-48 hours.

In some embodiments the reducing potential of the working electrode can be applied to a target area for 36-72 hours.

In some embodiments the reducing potential of the working electrode can be applied to a target area for 1-30 minutes.

In some embodiments the reducing potential of the working electrode can be applied to a target area for 30-90 minutes.

In some embodiments the reducing potential of the working electrode can be applied to a target area for 15-45 minutes.

In some embodiments the reducing potential of the working electrode can be applied to a target area for 1-180 minutes.

In some embodiments the reducing potential of the working electrode can be applied at pH different from 7, Determination of how the midpoint potential in the ranges of the disclosure changes when the pH changes can be performed as will be understood by a skilled person upon reading of the present disclosure (see e.g. Example 13 to 16).

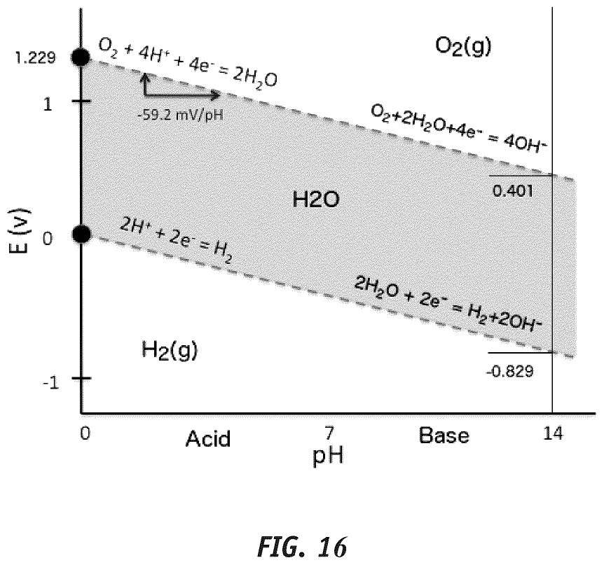

In general, adjusting the midpoint potential value from a starting pH (e.g. pH 7) to a different pH can be performed according to the Nernst equation discussed in Example 15. The voltage stability window of water must also be taken into account when operating under different potentials and pH conditions, as shown in the Pourbaix diagram in FIG. 16 and discussed in Example 16.

Accordingly and for added guidance if an exemplary target redox active compound has E_{1/2} from a 0 mV to −400 mV vs Ag/AgCl at pH7, and is in a target area with pH from 5 to 9 (such as in a wound) the difference in voltage can be adjusted accordingly as will be understood by a skilled person In particular, adjusting the E_{1/2} of an exemplary target redox active compound to pH 5 (e.g. by adding 59 mV*2 to the value at pH 7) results in an E_{U2} range from a 120 mV to −280 mV vs Ag/AgCl at pH5, thus a bacteria reducing voltage difference of 70 mV to −480 mV vs Ag/AgCl, with a preferred bacteria reducing voltage difference of 20 mV to −380 mV. Likewise, adjusting the E_{1/2} of the same exemplary target redox active compounds to pH 9 results in a range of −120 mV to −520 mV vs Ag/AgCl, with a bacteria reducing voltage difference range −170 mV to −720 mV; and preferred range −220 mV to −620 mV as would be understood by a skilled person.

A skilled person will be able to identify midpoint potential and corresponding reducing potential of the working electrode and negative voltage to be applied to the working electrode and the counter electrode relative to a reference electrode, which can be effective to target specific redox-active compounds and related producing bacteria the voltage window of water under physiological conditions for the bacteria as will be understood by a skilled person upon reading of the disclosure.

In some embodiments, the bacterial reducing voltage difference is applied to a medium with a configuration directed to maximize the effect of the voltage difference application. In those embodiments because the bacterial reducing voltage difference is a negative voltage difference the configuration of the electrodes is selected to locate the working electrode on a target areas of the medium where the bacterial concentration is high.

The number of working electrodes and the related configuration is thus selected in view of the type of electrodes, type of medium and the dimension area to be targeted noting that a single electrode can affect an area within a radius of up to 200 um, up to 500 μm or higher depending on the specific feature of the medium the type and the electrodes, the related configuration and the conditions under which the voltage is applied as will be understood by a skilled person.

In some embodiments the electrodes have an area from 100 um2 to 1,000,000 um2. In some embodiments the electrodes have an area from 1 mm2 to 100 mm2. In some embodiments the electrodes have an area from 1 cm² to 10 cm². The corresponding target areas in the medium depend on many factors including accessibility of chemicals such as O that can re-oxidize the redox compounds enabling the cells to respire which would compete against the effect of voltage application according to the present disclosure. Additional dimensions of the electrodes and related configurations can be identified by a skilled person in view of a corresponding target area of the medium to be targeted. Those dimensions can be identified by a skilled person upon reading of the present disclosure, in view of specific medium, target area, conditions of voltage applications and desired result of the setting. For example, in some embodiments multiple electrode pairs can be applied different target areas of a medium (e.g. a wound) to achieve a greater effect than the effect reachable by a single electrode pair for a given target area of the medium.

Additional, combination of electrode areas, corresponding target areas and timing of application can be identified by a skilled person upon reading of the present disclosure in view of the effect on the viability of the bacteria desired according to the experimental design.

For example, for a working electrode such as the electrode schematically shown in FIG. 15 with a working electrode of about 6 cm², a counter electrode of about 2 cm² separated from each other by a distance between electrodes about 1 mm, a 10-100 μA/cm² current density, applied over 24 hours to a medium in physiological conditions, generates a steady-state concentration of a reduced redox active target compounds around 5-10 μM. In this exemplary embodiment, high concentration areas persist around 200 μm from the electrodes as will be understood by a skilled person.

In certain embodiments, the electrodes are less than 1 cm² and they are separated by 100 μm, and current is applied over 24 hours to generate a steady-state concentration of reduced redox-active target compounds around 1-5 uM, persisting around 50 μm from the electrodes.

In certain embodiments, the electrodes are less than 100 um² and they are separated by 10 μm, and current is applied over 72 hours to generate a steady-state concentration of reduced redox-active target compounds around 1-5 uM, persisting around 5 μm from the electrodes.

Detection of the reduction of the redox active compound and inhibition of viability of the bacteria can be performed with various methods identifiable by a skilled person.

In some embodiments the reduction of the target redox active compound can be detected through light absorption as will be understood by a skilled person.

In some embodiments, the redox-active compound has at least one oxidation absorption maximum in the wavelength of 400 nm to 700 nm in the oxidized state with a corresponding oxidation extinction coefficient. In some embodiments, the redox-active compound has at least one reduction absorption maximum in the wavelength of 400 nm to 700 nm in the reduced state with a corresponding reduction extinction coefficient.

In some embodiments, the at least one oxidation absorption maximum and the at least one reduction absorption maximum have an absorption shift or difference of at least 5 nm, preferably 50 nm and most preferably 150 nm.

In some embodiments, the ratio of oxidation extinction coefficient to the reduction extinction coefficient is at least 2 to 1, preferably at least 20:1 and most preferably 100:1.

In some embodiments, the ratio of reduction extinction coefficient to the oxidation extinction coefficient is at least 2 to 1, preferably at least 20:1 and most preferably 100:1.

Viability of at any point of the methods and systems of the disclosures can be detected with methods identifiable by a skilled person such as detecting colony forming units (CFU) when plates on agar growth medium, ability to grow in liquid medium measured by changes to optical density or observation under a microscope, staining a vitality stain such as Syto9 or a tetrazolium dye that interacts with the electron transport chain and gets reduced inside the cell (e.g. CTC) and additional methods identifiable by a skilled person. Also Reduced phenazines have characteristic fluorescent spectra (see e.g. [22]) and can also be detected by additional methods identifiable by a skilled person.

In preferred embodiments, electrochemical control of phenazine redox cycling to bring to a reduced state phenazines in medium containing phenazine-producing bacteria and in particular when the medium comprises bacterial biofilms, can be combined with treatment with an antibiotic or other antimicrobial to further decrease viability of cells.

In particular, in most preferred embodiments treating the redox active bacteria with antibiotics and or other antimicrobial at a time and under condition when the redox active compound (e.g. phenazines) are in the reduced state can results in a decreased viability up to killing cells in a biofilm which can be 100-fold more effective if compared with other methods directed to obtain electrochemical control of bacteria viability (see Examples 1 to 3).

An “antimicrobial” as described herein indicates a substance that kills or inhibits the growth of microorganisms such as bacteria, fungi, or protozoans. Antimicrobial either kills microbes (microbiocidal) or prevent the growth of microbes (microbiostatic).

Microbicidal (also identified as bactericides) and microbiostatic (also identified as bacteriostatic agents) comprise disinfectants which are chemical substances or compound used to inactivate or destroy microorganisms on inert surfaces. Exemplary disinfectants comprise alcohols aldheydes oxidizing agents peroxy and peroxo acids, phenolics, quaternary ammonium compounds, inorganic compounds and additional disinfectants identifiable by a skilled person.

Bactericides and bacteriostatic agents also comprise antiseptics, which antimicrobial substances or compound that are applied to living tissue to reduce the possibility of sepsis, infection or putrefaction. Exemplary antiseptics comprise alcohols, iodine, diguanides, peroxides, phenols and other disinfectants identifiable by a skilled person.

Bactericides and bacteriostatic agents also comprise further comprise antibiotics.

The term “antibiotics” as used herein refers to a type of antimicrobial used in the treatment and prevention of bacterial infection. Some antibiotics can either kill or inhibit the growth of bacteria. Others can be effective against fungi and protozoans. The term “antibiotics” can be used to refer to any substance used against microbes. Antibiotics are commonly classified based on their mechanism of action, chemical structure, or spectrum of activity. Most antibiotics target bacterial functions or growth processes. Antibiotics having bactericidal activities target the bacterial cell wall, such as penicillins and cephalosporins, or target the cell membrane, such as polymyxins, or interfere with essential bacterial enzymes, such as rifamycins, lipiarmycins, quinolones and sulfonamides. Antibiotics having bacteriostatic properties target protein synthesis, such as macrolides, lincosamides and tetracyclines. Antibiotics can be further categorized based on their target specificity. “Narrow-spectrum” antibacterial antibiotics target specific types of bacteria, such as Gram-negative or Gram-positive bacteria. “Broad-spectrum” antibiotics affect a wide range of bacteria.

In some embodiments, suitable antibiotics that can be used in the antimicrobial in combination with Fe chelators include ampicillin, kanamycin, ofloxacin, Aminoglycosides, Carbapenems, Ceftazidime, Cefepime, Ceftobiprole, Fluoroquinolones, Piperacillin, Ticarcillin, tobramycin, aztreonam, coliston, tazobactam, and others (or combinations of these antibiotics) that can be recognized by a person skilled in the art.

In some embodiments, suitable antibiotics comprise antibiotics effective against pathogen Pseudomonas aeruginosa such as Aminoglycosides, Carbapenems, Ceftazidime, Cefepime, Ceftobiprole, Fluoroquinolones, Piperacillin, Ticarcillin, tobramycin, aztreonam, coliston, and others (alone or in combination) that can be recognized by a skilled person.

Exemplary antibiotics that can be used in combination with the bacterial reducing voltage difference herein described comprise Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. As a person of ordinary skill in the art would understand, the antibiotics herein listed can be selected for treating infections or reducing inflammation caused by bacteria including Staphylococcus aureus, Pseudomona (P. aeruginosa), Burkholderia cepacian, some mycobacteria.

Additional antibiotics suitable in particular for treatment of cystic fibrosis include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. A person skilled in the art would be able to select appropriate antibiotics for treating cystic fibrosis caused by particular pathogen. An exemplary indication of antibiotic, is shown in Table 5 below From Orenstein, D. Cystic Fibrosis: A Guide for Patient and Family, 4th ed. LWW; 2011. [36]

TABLE 5
An exemplary list of antibiotics

| | | |
|---|---|---|
| Type and kinds | Bacteria Treated | How Taken |

Penicillins

| | | |
|---|---|---|
| Amoxicillin and clavulanic acid (Augmentin ®) | Staphylococcus aureus (Staph) | |
| Methicillin, oxacillin and nafcillin | Pseudomonas (P. aeruginosa) | Intravenous, |
| | | intramuscular |
| Cloxacillin and dicloxacillin | Staph | Oral |
| Cabenicillin, ticarcillin, piperacillin, mezlocillin | P. aeruginosa | Intravenous |
| and azlocillin | | |
| Ticarcillin and clavulanic acid (Timentin ®) | Staph, P. aeruginosa | Intravenous |
| Piperacillin and tazobactam (Zosyn ®) | P. aeruginosa | Intravenous |

Cephalosporins

| | | |
|---|---|---|
| Cephalexin, cefdinir, cefprozil and cefaclor | Staph, P. aeruginosa | Oral |
| Cefuroxime | Staph | Oral |

Sulfa

| | | |
|---|---|---|
| Sulfisoxazole | P. aeruginosa | Oral |
| Erythromycin/sulfisoxazole | Staph | Oral |

Aminoglycosides

| | | |
|---|---|---|
| Tobramycin, amikacin, gentamicin | P. aeruginosa (in combination | Intravenous, inhaled |
| | with gentamicin, tobramycin, and | |
| | amikacin; also work well with | |
| | anti-Pseudomonas penicillin | |
| | drug) | |

Macrolides

| | | |
|---|---|---|
| Erythromycin, clarithromycin and azithromycin | Staph and may help reduce | Oral, intravenous |
| | inflammation from P. aeruginosa | |

Tetracyclines

| | | |
|---|---|---|
| Tetracycline, doxycycline, minocycline, and | Formerly P. aeruginosa, some | Oral, intravenous, |
| tigecycline | Burkholderia cepacian and Staph | intramuscular |

Quinolones

| | | |
|---|---|---|
| Ciprofloxacin, levofloxacin | Pseudomonas | Oral, intravenous |

Vancomycin

| | | |
|---|---|---|
| Vancomycin | Staph and methicillin-resistant | Intravenous |
| | Staphylococcus aureus (MRSA) | |

Linezolid

| | | |
|---|---|---|
| Linezolid | MRSA and some mycobacteria | Oral, intravenous |

Imipenem & Meripenem

| | | |
|---|---|---|
| Imipenem & Meripenem | P. aeruginosa, Staph | Intravenous |

Aztreonam (Cayston ®)

| | | |
|---|---|---|
| Aztreonam (Cayston ®) | P. aeruginosa | Intravenous, inhaled |

In particular, in preferred embodiments, application of the negative difference in voltage has been performed in combination with antibiotic treatment of the redox active bacteria while the medium provides a reductive environment for the bacteria and the redox active compound is in a reduced state.

In some embodiments, the antibiotic and/or other antimicrobial can be administered simultaneously in combination or in sequential way with respect to the application of the bacterial reducing voltage difference.

In particular, in preferred embodiments the timing of administration can be selected to maximize the concentration of antibiotic and/or antimicrobial when the amount of redox active compound in a reduced state is maximized in a target area of the medium.

In particular, in some embodiments, antibiotics and/or antimicrobials can be applied at any stage with respect to the electrochemical treatment, before, during, after application of the voltage. In particular antibiotics and/or additional antimicrobial are administered to the medium for a time and under condition to allow presence of the antibiotic and/or antimicrobial when the voltages are applied. The specific timing depends on the specific feature of the medium, the antibiotics and/or antimicrobial administered as well as administration conditions as will be understood by a skilled person. In preferred embodiments the antibiotic is add simultaneously with the voltage application or preferably at a time preceding the voltage application so that antibiotic and/or other antimicrobials are present and do not diffuse before the voltage is applied as will be understood by a skilled person upon reading of the present disclosure.

In some embodiments, the antibiotic and/or other antimicrobial can be administered for a time selected to maximize the killing of bacteria up to complete eradication of the bacteria from the target area of the medium.

In particular, in some embodiments, antibiotics and/or antimicrobials can be applied for a time resulting in the desired inhibition of the bacteria typically resulting in the eradication of the bacteria and in case the method is performed to treat an infection for a time resulting in the treatment and/or prevention of the infection as will be understood by a skilled person.

In embodiments herein described an antibiotic can be administered to the medium known or suspected to contain phenazine producing bacteria at any concentrations suitable to inhibit viability of bacteria in a reduced medium which are identifiable by a skilled person upon reading of the present disclosure.

In particular, in preferred embodiments antibiotics can be administered at a concentration below the minimum inhibitory concentration (sub-MIC) which can added to the electrochemically controlled medium where phenazine, if any is present, is present in a reduced state.

Accordingly in embodiments herein described concentration of suitable antibiotics that can be used in the antimicrobial against phenazine producing bacteria can identified based on the respective breakpoint Minimum Inhibitory Concentration (MIC).

The wording breakpoint minimum inhibitory concentration (MIC) indicates the concentration that inhibits visible bacterial growth at 24 hours of growth in specific media, at a specific temperature, and at a specific carbon dioxide concentration. Methods that can be used to measure the MIC of a microorganism comprise broth dilution, agar dilution and gradient diffusion (the ‘E test’), where twofold serial dilutions of antibiotic are incorporated into tubes of broth, agar plates or on a paper strip, respectively, as will be understood by a person skilled in the art. The disk diffusion method defines an organism as susceptible or resistant based on the extent of its growth around an antibiotic-containing disk. MIC values are influenced by several laboratory factors.

Laboratories follow standard for parameters such as incubation temperature, incubation environment, growth media, as well as inoculum and quality control parameters. In the U.S. Standards for determining breakpoint MIC values for various bacteria can be found in Clinical & Laboratory Standards Institute (CLSI) publications, with an example also provided as Appendix A of U.S. Provisional Application No. 62/722,124 incorporated herein by reference in its entirety, as will be understood by the skilled person. In Europe, standards for determining breakpoint MIC values for bacteria can be found in European Committee on Antimicrobial Susceptibility Testing (EUCAST) see www.eucast.org/clinical_breakpoints/dated March 2023 and at the time of filing of the instant disclosure) as will be understood by the skilled person.

In some embodiments, in methods and systems herein described and related compositions one or more antibiotics can be administered in concentration of at least 0.00005 ug mL, preferably at least 0.002 ug mL, at least 0.01 ug mL, at least 0.025 ug mL, or at least 0.08 ug mL, or at least 0.1 ug mL, and in additional concentrations identifiable by a skilled person upon reading of the present disclosure. The specific concentration of each antibiotic can be determined based on the related MIC as will be understood by a skilled person.

In most preferred embodiments of methods and systems of the present disclosure, one or more antibiotics can be administered at a concentration of at least 2.0 ug mL, at least 10.0 ug mL, at least 25.0 ug mL, at least 50.0 ug mL, and at least 100.0 ug mL-1, in particular in combination with concentration of one or more bacterial reducing voltage difference in a concentration associated with a resulting synergic inhibition of bacteria viability herein described.

In some preferred embodiments the antibiotic can comprise one or more aminoglycosides (e.g. tobramycin, gentamicin), fluoroquinones (e.g. ciprofloxacin), beta lactams (e.g. ampicillin), polymyxins (e.g. colistin) and additional antibiotics identifiable by a skilled person.

The specific concentration of each antibiotic can be determined based on the related MIC as will be understood by a skilled person.

In most preferred embodiments, antibiotics used in methods and systems and related compositions of the present disclosure are aminoglycosides. The term “aminoglycosides” as used herein indicates an antibiotic that inhibit protein synthesis and contain an amino-modified glycoside aa portion of the molecule. Aminoglycoside antibiotics are typically used as a Gram-negative antibacterial medication, more typically against Gram-negative aerobes. such as Pseudomonas, Acinetobacter, and Enterobacter as well as some Mycobacteria, including the bacteria that cause tuberculosis, as well be understood by a skilled person.

Aminoglycosides antibiotics can be categorized based on the molecular structure in 4,6-disubstituted deoxystreptamine sub-class of aminoglycosides, the neomycins are examples of the 4,5-disubstituted sub-class, and a non-deoxystreptamine aminoglycoside subclass. Aminoglycosides antibiotics are typically administered intravenously and intramuscularly, topical preparations for wounds, oral administration for gut decontamination (e.g., in hepatic encephalopathy) and/or a nebulized form.

Exemplary aminoglycoside antibiotics comprise, Kanamycin A Amikacin, Tobramycin, Dibekacin, Gentamicin, Sisomicin, Netilmicin, Neomycins B, C, Streptomycin and Plazomicin wherein kanamycin A through netilmicin are examples of the 4,6-disubstituted deoxystreptamine sub-class of aminoglycosides, the neomycins are examples of the 4,5-disubstituted sub-class, and streptomycin is an example of a non-deoxystreptamine aminoglycoside. Tobramycin is an exemplary representative of aminoglycosides as will be understood by a skilled person.

In some preferred embodiments the antibiotics can be selected from the group consisting of gentamicin, meropenem, ciprofloxacin, and colistin, and can include gentamicin.

Methods for detecting and evaluating the viability of bacteria after the use of the methods and systems for interference with viability of bacteria described herein include, but are not limited to, measurement of colony forming units, cell counts such as that described by Wang et al. [31], and other methods identifiable to a skilled person upon the reading of the present disclosure.

The terms “detect” or “detection” as used herein indicates the determination of the existence, presence, or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred to as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

Detection of the bacteria to verify the result of the method can be performed with viability assays testing the ability to culture the pathogen on a standard agar plate, and/or with growth independent test such as performing quantitative PCR to detect on biological markers of the bacteria, performing diagnostic mass spectrometry or additional techniques identifiable by the skilled person.

In some embodiments, the methods and systems and related devices and compositions here described can be used to inhibit pathogenic microbial biofilm formation as well as to disrupt mature biofilm in vitro and in vivo (see Examples 1 to 3).

In particular, in some embodiments, the methods and systems and related devices and compositions here described can impact early stages in biofilm formation and development by decreasing the biofilm surface coverage.

In some embodiments, the methods and systems and related devices and compositions here described can disrupt the mature biofilm by interfering with anoxic growth of pathogens in deeper layers of the biofilm.

A person skilled in the art would understand that as biofilm matures, cells in deeper layers of the biofilm begin to experience oxygen limitation and redox stress, rendering the cells to be slow growing and highly resistant to antibiotics.

Thus, in some embodiments, a method for inhibiting bacteria biofilm formation and/or disrupting mature biofilm in a medium is described, the method comprising applying a reducing voltage to the biofilm or to the medium comprising the biofilm. The suitable medium comprises growth medium or culture medium in a liquid or gel designed to support the bacteria in vitro, as well as tissues and other suitable environments within a host (including a human host) in vivo.

In embodiments, the determination of a proper concentration of antibiotics to inhibit reduce and/or kill bacteria according to methods of the disclosure can be performed in combination with clinical concentrations of antimicrobials, which differ depending on the antimicrobial agent.

In some embodiments, an antimicrobial can comprise phenazine degrading agents such as the agents described in U.S. Ser. No. 10/913,936, and US-2022-0175892, Fe chelating agents described in U.S. Pat. No. 9,926,562, U.S. Ser. No. 10/689,713, U.S. Ser. No. 10/406,211, and U.S. Ser. No. 11/820,973, as well as chlorate in absence of at least one preferably all other chlorite oxyanion at least for Pseudomonas and Nar-containing bacteria described in US-2019-0142864 and in US-2021-0322462 the content of each of which is incorporated by reference in its entirety. The antimicrobial can be administered and/or comprised in a composition in an amount suitable to reduce antibiotic resistance and/or survivability of phenazine producing bacteria. In some embodiments, the antimicrobial can comprise a compatible vehicle, which can be a vehicle for effective administrating and/or delivering of the one or more agents to an individual. In some embodiments of the methods and systems, the bacteria comprise persister cells.

In some embodiments, the antibiotics and/or antimicrobials can be comprised within a pharmaceutical composition comprising the antimicrobials herein described one or more antibiotics and/or an additional antimicrobial.

In some embodiments, a composition can comprise one or more antimicrobials herein described with one or more medium components.

In some embodiments, the antimicrobial can comprise one or more antimicrobials optionally a compatible vehicle for effective administrating and/or delivering of the one or more agents to an individual.

The term “vehicle” as used herein indicates any of various media acting usually as solvents, carriers, binders or diluents for antibiotics and/or additional antimicrobials comprised in the composition as an active ingredient.

In some embodiments, antimicrobial is a pharmaceutical composition comprising one or more antimicrobials for the treatment of cystic fibrosis and a pharmaceutically acceptable vehicle such as an excipient or diluent.

The term “excipient” as used herein indicates an inactive substance used as a carrier for the active ingredients of a medication. Suitable excipients for the pharmaceutical compositions herein disclosed include any substance that enhances the ability of the body of an individual to absorb the one or more agents. Suitable excipients also include any substance that can be used to bulk up formulations with the one or more agents to allow for convenient and accurate dosage. In addition to their use in the single-dosage quantity, excipients can be used in the manufacturing process to aid in the handling of the one or more agents. Depending on the route of administration, and form of medication, different excipients may be used. Exemplary excipients include but are not limited to anti-adherents, binders, coatings disintegrants, fillers, flavors (such as sweeteners) and colors, glidants, lubricants, preservatives, or sorbents.

The term “diluent” as used herein indicates a diluting agent which is issued to dilute or carry an active ingredient of a composition. Suitable diluents include any substance that can decrease the viscosity of a medicinal preparation.

In particular, in some embodiments, antimicrobials herein described herein described can be included in pharmaceutical compositions in combination with one or more compatible and pharmaceutically acceptable vehicles, and in particular with pharmaceutically acceptable diluents or excipients. In those pharmaceutical compositions, the antimicrobial and in particular one or more antibiotics, can be administered as an active ingredient for treatment or prevention of a condition in an individual.

In some embodiments, methods and systems, and related compositions and devices herein described can be used in methods for treating and/or preventing a bacterial infection by a phenazine producing bacteria in an individual.

The term “treatment” as used herein indicates any activity that is part of a medical care for, or deals with, a condition, medically or surgically.

The term “prevention” as used herein indicates any activity which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “condition” as used herein indicates a physical status of the body of an individual (as a whole or as one or more of its parts), that does not conform to a standard physical status associated with a state of complete physical, mental and social well-being for the individual. Conditions herein described include but are not limited to disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The term “individual” as used herein in the context of treatment includes a single biological organism, including but not limited to, animals and in particular higher animals and in particular vertebrates such as mammals and in particular human beings.

The term bacterial infection as used herein indicates a condition where tissue, organs and/or biofluids of the individuals are infected by a bacteria, in particular a redox active bacteria such as phenazine producing bacteria. Exemplary bacterial infection in the sense of the disclosure comprises infections of the eye (e.g., infection by contact lenses having biofilm), the lung (e.g., chronic lung infections), the gastrointestinal tract, the gum and/or jawbone (periodontal tissue), the internal tissue (e.g., endocarditis) and the skin (e.g., infected skin, infected burn Wounds) of tan individual.

The term “wound” as used herein indicates the result of a disruption of normal anatomic structure and function of an individual [37][38]. Accordingly, wounds in the sense of the disclosure encompass a wide range of a defects or breaks in a tissue and/or organs of an individual, resulting from physical, chemical and/or thermal damage, and/or as a result of the presence of an underlying medical or physiological condition” as will be understood by a skilled person [39].

Exemplary wounds comprise abrasions and tears of a tissue of an organ of an individual (e.g. skin) which can be caused by blunt and/or frictional contact with hard surfaces, such as when the an organ is torn, cut, or punctured (an open wound), when the organ is contused (a closed wound), as well as when the organ lesioned and comprise a region in an organ or tissue having abnormal structural change, e.g. following damage through injury or disease. [39]

Exemplary wounds comprise ulcers, like decubitis ulcers (bedsores or pressure sores) and leg ulcers (venous, ischaemic or of traumatic origin). [40], [41], [42], abscesses such as lesions caused by foreign bodies at the time of an injury, or by infections and tumors [39].

In particular wounds comprise abnormal structures in the body of an individual caused by mechanical forces (such as knives and guns but also surgical treatment), thermal sources, chemical agents, radiation, electricity and/or other sources identifiable by a skilled person [39][43]. Wounds also comprise abnormal anatomic structure and function of organs and/or tissues in an individual resulting from conditions such as autoimmune diseases or disorders, infections such as viral infections, cancer, as well as chronic diseases such as diabetes.

Exemplary wounds comprise superficial wounds (affecting only a surface epithelium of the organ, e.g. epidermal skin), partial thickness wounds (also affecting a connective tissues, of the organ such as skin's deep dermal layers) and full thickness wound (further affecting deeper tissues of the organ such as subcutaneous fat in addition to the epidermis and dermal layers) [39]; [44][45].

Exemplary wounds also comprise lesions in eyes, ears, stomach intestine and additional portions of the gastrointestinal tract, and in additional tissue organ or body part, including lesions occurring in pulmonary infections such as cystic fibrosis and additional conditions, and in general to chronic infections such as the ones associated with implanted medical devices in lungs and additional tissues and organs of an individual.

In some embodiments, the method for treating and/or preventing a bacterial infection in an individual comprises applying to the individual an effective a bacterial reducing voltage difference the administering performed to target the medium in the individual comprising and/or suitable to comprise redox active bacteria such as phenazine producing bacteria. In the method the applying is performed alone or in combination with administration of an antibiotic and/or other antimicrobial. In particular, the bacterial reducing voltage difference herein described alone or in combination with antibiotic administration, will be selected by the skilled person as not interfering in a deleterious manner with the normal biochemical pathways of the individual.

The administering to the individual the bacterial reducing voltage difference herein described can be performed to teat bacterial infections.

In embodiments, herein described the difference in voltage effective to provide a bacterial reducing voltage difference can be applied alone or in combination with an antibiotic and/or other antimicrobial performed in turn through various administration routes including oral ingestion, inhalation, intranasal, topical application, intravenous or subcutaneous injections and others as will be recognized by a person skilled in the art. The antibiotic and/or other antimicrobial can be provided in a form of an aqueous solution, cream, solid powder, tablets, aerosols, or other forms as will be understood by a person skilled in the art.

In particular, antimicrobials that can be administered to a subject to treat in the subject infections associated with a phenazine producing bacteria are antimicrobial that have been further selected through preclinical and clinical studies to assess the relevant efficacy, safety (pharmacovigilance), tolerability, pharmacokinetics, and pharmacodynamics in the subject before administration. Those tests and trials include but are not limited to in vitro and in vivo tests and studies, first-in-human-trials, Single Ascending Dose studies (SAD), Multiple Ascending Dose studies (MAD studies), trials designed to investigate any differences in absorption of the inhibitor by the body, caused by eating before the inhibitor is given and other trials established by the U.S. Food and Drug Administration and identifiable by a skilled person.

In some embodiments of the treatment methods, the bacterial infection is caused by a pathogen of the Actinobacteria or Proteobacteria phyla. In some embodiments, the bacterial infection is caused by a pathogen of the Pseudomonas genus. In some embodiments, the bacterial infection is caused by a Pseudomonas pathogen selected from the group consisting of: P. aeruginosa, P. oryzihabitans, P. fluorescens, and P. luteola. In other embodiments, the bacterial infection is caused by a pathogen of the Streptomyces genus. In other embodiments, the pathogen is resistant to beta-lactam antibiotics, penicillin, piperacillin, imipenem, tobramycin, or ciprofloxacin.

As used herein, treatment can be prophylactic (e.g., to prevent or reduce the risk of an infection) or therapeutic or curative. Accordingly, subjects to be treated may be subjects that are infected or subjects that are at risk of infection. Subjects at risk of infection may be immuno-compromised subjects or subjects that have a condition that makes them susceptible to infection by one or more organisms (e.g., bacterial pathogens) described herein. For example, a subject at risk of infection may be a subject that has an HIV infection, AIDS, Cystic Fibrosis, or other disease or condition that causes an immunodeficiency. In some embodiments, a subject at risk of infection can be a subject that has been wounded (e.g., suffered a cut or other wound) or a subject that is undergoing or has undergone surgery. A subject having an infection may be a subject infected with one or more organisms described herein. An infection may be a systemic infection or a wound infection (e.g., at the site of a cut or abrasion, including for example, at the site of a surgical incision) or any other type of infection (for example, any infection where anaerobic conditions may exist or prevail at the site of infection).

Accordingly, a reducing potential and any compound herein described can be administered to any suitable subject prior to, during, or after infection (or prior to, during, or after exposure to a disease, condition, accident, or procedure that exposes the subject to a risk of infection).

In some embodiments, the reducing potential can be applied with a device can be provided which can be used in methods and systems of the disclosure.

In particular, in some embodiments a device for decreasing viability of a target redox active bacteria in a medium is described, which comprises
a voltage source a working electrode a counter electrode and a reference electrode configured to present the working electrode for contact with a target area of the medium known or expected to comprise a target redox bacteria (if any)
the voltage source, configured to operate with the working electrode the counter electrode and the reference electrode, and to provide a reduction potential of the wording electrode relative to the reference electrode, lower than the target midpoint potential of a target redox compound produced by the redox bacteria.

In preferred embodiments the device further comprises
at least one antimicrobial source, configured to release one or more antibiotic and/or other antimicrobials when the concentration of the redox active compound in the reduced state is higher than the concentration of the redox active compound in an oxidized state. In particular, in some embodiments the release is timed to occur before, simultaneously or after the working electrode potential is lower than the midpoint potential depending on the experimental design.

In some embodiments, the device can be an electrically controllable device configured to provide an electrical current to a medium known or suspected to comprise a target redox active bacteria producing a target redox compound. In particular, the device can comprise: a voltage source, in a first section of the device, configured to generate a reducing potential of a working electrode in contact with the medium and thus convert the target redox compound to a reduced state. The electrically controllable device can also include an antibiotic source, in a second section of the device, configured to release antibiotics, when the reducing potential of the working electrode is lower than the midpoint potential of the target redox compound and thus when the redox active compound is in the reduced state.

In some embodiments an electrically controllable device of the disclosure can be wearable electrically controllable bandage configured to be worn on a target medium of an individual host known or suspected to comprise redox active bacteria producing a target redox compound, the bandage comprising:
a voltage source a working electrode a counter electrode and a reference elected, in a first section of the bandage, configured to generate a reducing potential on the working electrode lower than the midpoint potential of the target redox compound to increase the electronical conversion of target redox compound into a reduced state; and
an antibiotic source, in a second section of the bandage, configured to release antibiotics while concentration of the target redox compound in the reduced state in the medium is higher than the concentration of the target redox active compound in an oxidized state.

Accordingly in some embodiments the antibiotic source is configured to release the antibiotic simultaneously in combination or sequentially with respect to the generation of the reducing potential to maximize the concentration of antibiotic in the medium when the concentration of the target redox compound in the reduced state is higher than the concentration of the target redox compound in an oxidized state.

In the devices herein described the configuration of the electrodes is preferably set up to maximize the contact between the working electrode and a target area of the medium where the redox active compound is known or expected to be present (e.g. an infected are, including infected area comprising biofilm). Accordingly, the wearable electrically controllable bandage can comprise
working electrode connected to the voltage source; and
a counter electrode connected to the voltage source;
   wherein the working electrode is configured with the voltage source to be at a lower potential than the counter electrode and is configured to be in contact with selected portion of a body of the host individual when in use.

In devices in the sense of the disclosure a reference electrode configured to measure the voltage between the working electrode and the counter electrode.

In devices in the sense of the disclosure the working electrode and the counter electrode can be part of an array of electrodes with a plurality of working electrode and a plurality of counter electrodes in an alternating pattern.

The methods described herein can be performed by devices, or systems of devices, as one skilled in the art would understand. The principle of operation would be to have an electrode (“working electrode”) placed on a surface or in a medium that contains a bacteria that is to be subject to anti-bacterial agents, the bacteria using a redox active compound for electron transfer. The working electrode is brought to a negative voltage potential (compared to a reference point, such as a reference electrode) that is more negative than the mid-point potential of the redox active compound, but not to the point that the integrity of the surface/medium is compromised. Therefore, any device or system that can realize this would be useable in performing this method. Typically, such a device or system would include a power source (battery, outlet, generator, capacitor, etc.) capable of creating the desired voltage level for the desired length of time (as described herein), a working electrode configured to be applied to the surface or medium in question, a counter electrode to complete the circuit with the working electrode, and a reference electrode for ensuring the working electrode is at the desired potential. The device or system can also include controls for setting the voltage, handles for manipulating the electrodes, other control/sensor circuitry, etc. The device or system can also incorporate a mechanism to apply the anti-bacterial agent to the surface/medium, such as a sprayer, a disperser, a package-release chip, or other liquid/gel application device. The devices or systems can be purpose-built to primarily sanitize another object/surface/tissue, or it can be incorporated into something with a different primary purpose, but includes a self-sanitizing capability (e.g., an artificial hip that includes the working electrode for maintaining an anti-bacterial surface).

Examples of such devices and system can include hand-held sanitizing tools or medical instruments, medical implants, electronic bandages, sanitizing surfaces, sanitizing tanks, self-sanitizing aeration systems, self-sanitizing A/C devices, etc. Other uses would be known to those having skill in the art.

In some embodiments the device of the disclosure can be part of a medical implant where the voltage source the working electrode, the counter electrode and the reference electrode are configured to present the working electrode on a surface subjected to infection by a redox active bacteria producing a redox active compound in the sense of the disclosure. In the medical implant the voltage source the working electrode, the counter electrode and the reference electrode are configured to provide the working electrode with a reducing potential lower than the midpoint potential of the redox active compound for a time and under condition resulting in inhibition of the viability of the redox active bacteria in accordance with the present disclosure.

Accordingly in preferred embodiments the medical implant can also comprise at least tone antimicrobial source configured to release one or more antibiotic simultaneously in combination or sequentially with respect to the application of the reducing potential to the working electrode. In the medical implant the at least one antimicrobial source is configured to release one or more antimicrobial in sufficient concentration to further inhibit the viability of the redox active bacteria. In preferred embodiments, the antimicrobial comprises an antibiotic release in sub-MIC concentration.

In some embodiments the medical implant can comprise a coil for receiving wireless power from an external source; a working electrode on the surface of the medical implant connected to the coil; and a counter electrode connected to the coil. In those embodiments the working electrode and the counter electrode are configured to generate a phenazine reduction potential to bring phenazines to a reduced state when energized by the coil.

Additional devices and configurations can be identified by a skilled person upon reading of the present disclosure.

In some embodiments existing devices can be modified in accordance with the teachings of the present disclosure. For example, electroceutical bandages, where electrical field are controlled through local voltage sources in a first section of the bandage including, for example, electro-couples or variable potentiostats, can be configured to generate a reduction potential on the working electrode for a certain amount of time and then allow antibiotic release (through e.g. an antibiotic source in a second section of the bandage) and consequent treatment as soon as the working electrode reaches the reducing potential lower than the midpoint potential of a target redox compound.

Accordingly, for example, a electrically controllable wearable bandage configured to be worn on a host region with redox active bacteria, can be modified so that the bandage comprising: a voltage source, in a first section of the bandage, configured to generate a reducing potential to bring redox active compound in the host region to a reduced state; and an antibiotic source, in a second section of the bandage, configured to release antibiotics while the redox active compound is in the reduced state.

As described herein, voltage sources, electrodes, and antimicrobials herein described possibly within devices herein described can be provided as a part of systems to perform any methods, including any of the assays described herein to detect presence and/or viability of bacteria.

In particular in some embodiments a system to decrease viability of a target redox active bacteria in a medium in accordance with the present disclosure can comprise:
a voltage source operatively connected to a working electrode a counter electrode and a reference electrode, the voltage source configured to apply to the working electrode a working electrode potential relative to the reference potential lower than the target midpoint potential.

The system can further comprise a look up table reporting a set of redox active bacteria each accompanied by corresponding redox active compounds, midpoint potentials, a corresponding reducing working electrode potential as well as timing of application to obtain a set inhibition of bacteria viability under a set of operating conditions.

A look-up table as used herein is an N-dimensional array of data indexed by one or more input parameters, such that providing the input parameters provides the system with the data required for the solution (either the final solution, or an intermediate value used to derive the solution). Look-up tables can be stored in firmware or software. Look-up tables can be stored in memory locally, or they can be stored in a remote server where a request is sent to the remote server with the input parameters and the remote server returns the data accessed in the table. The look-up table can be populated by pre-calculating equations using the methods described herein.

The system can also comprise an antibiotic and/or other antimicrobial and/or related sources, for simultaneous combined or sequential use in the method to decrease viability of the of the redox active bacteria of the present disclosure.

In particular, in some embodiments where a look up table is comprised, a user can select a target the redox active bacteria, and corresponding target redox active compound and related midpoint potentials from the look up table. The user can then apply the corresponding working electrode potential for a selected time that result in a desired inhibition of the viability of the redox active bacteria in the medium.

In some embodiments, the system can take the form of a combination for sterilizing an area of interest, and comprise:
a voltage source, configured to generate a reducing potential in the working electrode to increase the conversion of redox active compound possibly present in the medium to a reduced state; and
a hand-held device connected to or comprising the voltage source, the hand-held device further comprising:
a working electrode connected to a first prong on the hand-held device; and
a counter electrode connected to a second prong on the hand-held device;
wherein the working electrode and the counter electrode are connected to the voltage source in a configuration such that the working electrode is at a lower potential than the counter electrode when in use.

In some embodiments, a system for sterilizing an area of interest, can further comprise a tube connected to or integrated in the hand-held device, the tube configured to deliver anti-bacterial material to the area of interest when the system is in use. In some embodiments, the working electrode is paddle shaped. In some embodiments, the hand-held device is configured in the form of medical forceps.

In some embodiments, the system in the sense of the present disclosure can be an aeration system comprising:
a tank;
an aeration tube configured to release air into the tank through at least one hole in the aeration tube;
a working electrode near the at least one hole;
a counter electrode near the at least one hole and near the working electrode;
wherein the working electrode and the counter electrode are configured to generate a phenazine reduction potential to bring phenazines to a reduced state.

In embodiments of systems herein described, the systems can be provided in the form of kits of parts, in particular when detection of viability of bacteria can be performed with components of the system.

In a kit of parts, the antimicrobial and the device as well as any other compositions and other reagents to perform the method can be comprised in the kit independently. In particular, the antimicrobial and/or one or more antibiotics, medium, bacteria and can be included in one or more compositions, and each together with a suitable vehicle. In some embodiments, a kit can comprise a voltage source with medium components within a composition herein described optionally further in combination with the antibiotic herein described. In some embodiments, a device of the disclosure can be comprised in addition or in the alternative to any one of the components indicated above.

In some embodiments the system can also comprise reagents for the detection of the viability of bacteria. In those embodiments, additional components can include labeled molecules and in particular, labeled antibodies, labels, microfluidic chip, reference standards, and additional components identifiable by a skilled person upon reading of the present disclosure. The terms “label” and “labeled molecule” as used herein as a component of a complex or molecule referring to a molecule capable of detection, including but not limited to radioactive isotopes, fluorophores, chemiluminescent dyes, chromophores, enzymes, enzyme substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemiluminescence, production of a compound in outcome of an enzymatic reaction and the like.

In some embodiments, detection of a viable bacteria can be carried either via fluorescent based readouts, in which the labeled antibody is labeled with fluorophore, which includes, but not exhaustively, small molecular dyes, protein chromophores, quantum dots, and gold nanoparticles. Additional techniques are identifiable by a skilled person upon reading of the present disclosure and will not be further discussed in detail.

The kit will normally contain the compositions in separate containers. Instructions, for example written or audio instructions, on paper or electronic support such as tapes, CD-ROMs, flash drives, or by indication of a Uniform Resource Locator (URL), which contains a pdf copy of the instructions for carrying out the assay, will usually be included in the kit. The kit can also contain, depending on the particular method used, other packaged reagents and materials (i.e. wash buffers and the like).

The methods and systems as well as related devices and compositions herein described, can be performed in vivo and/or in vitro as will be understood by a skilled person.

### EXAMPLES

The electrochemical inhibition of phenazine producing bacteria and related device compositions, methods and systems herein disclosed are further illustrated in the following examples, which are provided by way of illustration and are not intended to be limiting.

In particular, the following examples illustrate an exemplary proof of principle, of methods and protocols for electrochemical inhibition of phenazine producing bacteria and exemplary device expected to be able to deliver the bacterial reducing voltage difference to suitable medium in vivo.

A person skilled in the art will appreciate the applicability and the necessary modifications to adapt the features described in detail in the present section, to additional bacteria, voltage sources, bacterial reducing voltage difference, antibiotics, antimicrobials and related compositions, methods and systems according to embodiments of the present disclosure. The following materials and methods were used.

In experiments reported in this section, the following bacteria strains were selected and retrieved

TABLE 3
Strains used for this experimental section

| Bacterial strain | Description | Source |
|---|---|---|
| P. aeruginosa | DKN_WT | Schroth et |
| UCBPP-PA14 | | al., 2018 |
| P. aeruginosa | ΔphzA1-G1 ΔphzA2-G2 | Saunders et |
| UCBPP-PA14; Δphz* | ΔphzMS ΔphzH | al., 2020 |
| | (derivative of DKN_WT) | |

Bacterial growth. P. aeruginosa UCBPP-PA14 strains were plated on lysogeny broth (LB) agar from −80° C. stocks and incubated at 37° C. overnight. Plates were stored for a week at −4° C. Liquid cultures were grown on minimal medium (MM) containing succinate as carbon source and electron donor (14.15 mM KH₂PO₄, 38.85 mM K₂HPO₄, 42.8 mM NaCl, 9.3 mM NH₄Cl, 40 mM Na-succinate, 1× SL-10 trace element solution [46] adjusted to pH 7.2 and autoclaved, then 1 mM MgSO₄ was added) and were incubated while shaking at 37° C.

Electrochemical reactors and biofilm growth. Double-jacketed electrochemical reactors (Pine Instruments, 250 ml, wide center port 29/26) were used with ITO-coated 0.2 mm thick boro-aluminosilicate glass slides (Delta Technologies, 24×60 mm, Rs=7-10Ω) as working electrodes, freshly sanded graphite rods (Alfa Aesar) as counter electrodes, and Ag/AgCl in 3M KCl reference electrodes (BASi). Silver epoxy (Electron Microscopy Sciences) was used to make electrical connection between insulated wire (Digdegradinedegraddegi-key) and working electrode and was insulated using 5 Minute® expoxy (Devcon). Working electrodes were assembled within 5 hours of use. Reactors were sterilized by autoclaving without reference or working electrodes, which were sterilized by submerging in 10% bleach for 5 minutes or 30 seconds, respectively, and rinsed in sterile water. Individual mid-log liquid cultures (OD₆₀₀ 0.45-0.55) were used to inoculate sterile reactors containing 140 ml of MM (described above) with 0.1% (vol/vol) for each biological replicate. Antibiotics were not added at this point. Working electrodes were poised at either open circuit, +100 mV, or −400 mV vs. Ag/AgCl (+310 mV and −190 mV vs the standard hydrogen electrode, respectively). Reactors were aerated using an aquarium pump with a 0.2 μm filter (Thermo Scientific) and heated to 31° C. using a circulating water bath (Haake A 25, Thermo Scientific). Fresh medium was exchanged every 24 hours for 5 days. After 5 days of aerobic growth, working electrode-attached biofilms were rinsed in sterile medium to remove planktonic cells and transferred to sterile reactors with fresh MM previously flushed for 2 hours with N₂ ultra-high purity gas (NRL supply store). For antibiotic exposure experiments, antibiotics were added to sterile media before flushing with N₂. For PYO-addition experiments, 10 μM PYO (Cayman Chemical) was added to in the MM before flushing with N₂. Working electrodes were poised to their corresponding potential and biofilms were incubated for an additional 72 hours under N₂ flushing at 31° C.

Biofilm harvesting and processing. After 72 hours of incubation under anoxic conditions, electrode-attached biofilms were rinsed in sterile media. The biofilms within the bottom 3.12 cm² of each working electrode were scraped off using a cell scraper (Sarstedt) and resuspended in antibiotic-free MM. Serial dilutions were prepared in sterile antibiotic-free MM and plated on antibiotic-free LB. The rest of the electrode-attached biofilms were incubated at room temperature in freshly-made 2 μM TOTO-1 and 10 μM SYTO-60 (Invitrogen) in MM for 15 minutes and rinsed 2× in sterile MM for 15 minutes. Imaging was done with an upright Zeiss LSM 800 microscope with Airyscan and a 40× oil immersion lens. SYTO-60 was excited with a 561 nm; 0.20% laser and emission was recorded from 580-700 nm. TOTO-1 was excited with a 488 nm; 0.20% laser and emission was recorded from 490 to 580 nm.

Square Wave Voltammograms. Potential was scanned from +100 mV to −500 mV vs Ag/AgCl and in reverse with the following parameters: pulse height=50 mV, pulse width=66.6 ms, step height=−0.5 mV, and acquisition of the average current over the last 50% of each step. Square wave voltammograms were acquired immediately after transfer from oxic to anoxic conditions.

Biofilms on unpoised electrodes within oxidative/reductive reactors. Electrochemical reactors were prepared as described above, except that an additional ITO-covered glass slide identical to the working electrode was inserted through the central port, but it was never connected to the potentiostat. The wires of the working electrode and the unpoised slide were positioned so that the distance between them was ˜3 cm. Biofilms grown on unpoised slides were therefore subjected to the same diffusive conditions as the biofilms grown on poised working electrodes, but without direct contact with it. Both poised and unpoised electrodes were transferred together to anoxic reactors after 5 days of growth and processed at the same time after harvesting.

Biofilms harvested and plated in parallel oxic/anoxic media. For experiments analyzing cell survival as a function of time, aerobic biofilm growth and transfer to anoxic reactors was performed as described above. Time point t=0 corresponded to when working electrodes were re-poised to their corresponding potentials after transfer to anoxic conditions. At t=30 minutes, 6, 36, and 72 hours, anoxic electrochemical reactors were transferred into an anaerobic chamber (Whitley Workstation DG250) with a 7%:20%:73% H₂:CO₂:N₂ atmosphere where biofilm harvesting was performed as described above, except that the MM used to resuspend biofilms and prepare serial dilutions contained 2 g/L bicarbonate and was flushed with a 80%:20% N₂:CO₂ gas mix (Airgas) prior to sterilization and equilibrated inside the chamber for at least 24 hours. Anoxic LB plates were prepared with 40 mM KNO₃ as electron acceptor, pH was adjusted to 7.0, then 2 g/L bicarbonate was added before flushing with 80%:20% N₂:CO₂ and autoclaving. Plates were poured inside the anaerobic chamber and equilibrated for 24 hours before use. Oxic LB plates were brought into the anaerobic chamber for parallel plating, but were transferred back out immediately after plating and were incubated as described above.

Treatment of liquid cultures with reduced PYO. 100 μM PYO in MM was reduced electrochemically using a dual-chamber reactor with an cation exchange membrane (CMI-7000, Membranes International) to avoid re-oxidation of PYO at the counter electrode. Anodic chamber contained Ag/AgCl reference electrode, platinum mesh working electrode, and was flushed with N₂; while cathodic chamber was oxic and contained platinum mesh counter electrode. PYO reduction was carried out by poising the working electrode at −400 mV vs. Ag/AgCl for 24 hours, past the point of 100% PYO reduction based on coulombic efficiency, and biochemical reactor was then transferred into the anaerobic chamber. Anoxic vials with pre-equilibrated MM and corresponding concentrations of reduced PYO were then treated with biochemical O₂ scavenging system (10 mM glucose+375 nM glucose oxidase+750 nM catalase) for an hour before inoculation. Aerobic liquid cultures of P. aerughnosa in MM at mid-log (OD600=0.5) were spun down and resuspended in fresh media and used to inoculate assays with 10% (vol/vol). After 30 minutes, serial dilutions were prepared using anoxic MM and plated for CFU counts in LB+KNO₃ as described above.

### Example 1: A Negative Voltage Difference, but not a Positive Voltage Difference, Inhibits Survival in P. aeruginosa

Experiments have been performed showing that electrochemically disrupting redox cycling under anoxic conditions can inhibit cell survival.

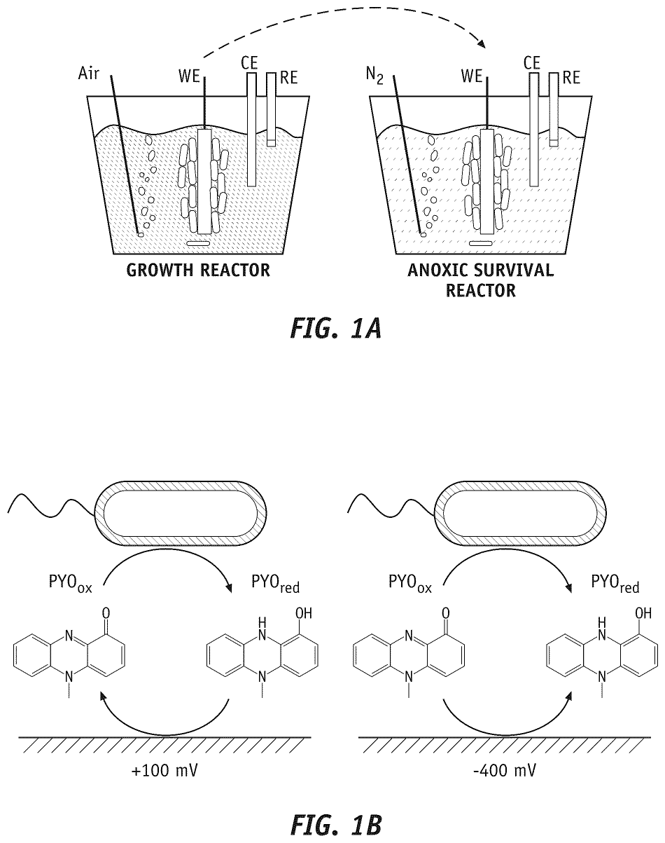

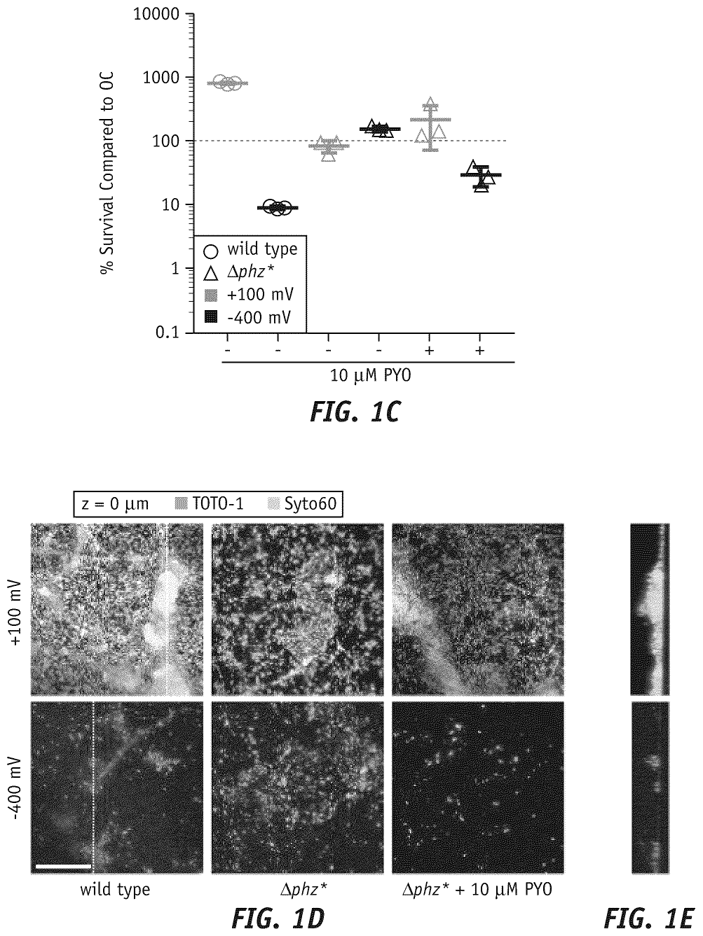

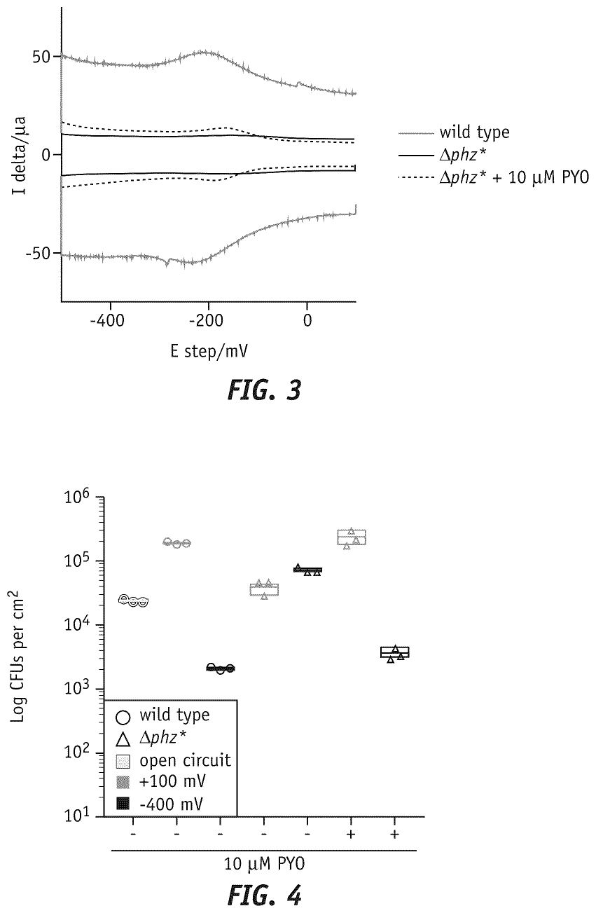

In order to test the effect of voltage difference on P. aeruginosa, P. aeruginosa PA14 biofilms were grown for 5 days in actively aerated three-electrode electrochemical reactors using an indium tin oxide (ITO)-covered glass slide as both a biofilm attachment surface and transparent working electrode (FIG. 1A). In contrast to previous studies ([47]), under the instant experimental conditions, the main phenazine detected was PYO (FIG. 3 and Experimental Procedures set forth above).

In FIG. 1A there are three electrodes: a working electrode, counter electrode, and reference electrode. The potential applied to the working is electrode −400 mV vs the Ag/AgCl reference electrode. The potential of the counter electrode is controlled by a potentiostat to ensure charge balance (i.e. source of electrons emitted by the working electrode. It is noted that the effect described by this disclosure can be achieved by two electrodes: the potential of the working electrode should be less than approximately −300 mV vs. Ag/AgCl. In some cases, the working electrode may be as high as −225 mV vs. Ag/AgCl. In this case the described disclosure will not perform as optimally as with the lower voltage, but will still have a stronger effect than no voltage applied.

Electrode-attached biofilms were then transferred to anoxic reactors for 72 h (FIG. 1A), after which the biofilms were harvested for colony forming unit (CFU) counts (FIG. 1C) and biofilm imaging (FIG. 1D). During both growth and after transfer to the anoxic reactors, the ITO working electrodes were poised at either the PYO-oxidative potential of +100 mV vs Ag/AgCl, or the PYO-reductive potential of −400 mV vs Ag/AgCl, which is not low enough to produce H₂O₂ in the presence of oxygen ([48]).

Under these conditions, for anoxic reactors in which the electrode was poised at +100 mV vs Ag/AgCl, PYO redox cycling can occur, but not in reactors in which the electrode was poised at −400 mV vs Ag/AgCl (FIG. 1B), except possibly at a low level if trace oxygen was present. Additional “untreated” control biofilms were set to open circuit (OC) in which no potential was applied to the electrode.

The results of these experiments show that a negative voltage difference but not a positive voltage difference inhibits survival in P. aeruiginosa. These results support the conclusion that the inhibitory effect of the electric current on survival of P. aeruginosa is obtained through block of pyocyanin redox cycle as illustrated more in detail in Example 2.

### Example 2: Electrochemically Blocking Pyocyanin Re-Oxidation During Anoxic Conditions Decreases Cell Survival

Electrical current was applied to P. aeruginosa PA4 in anoxic reactors in particular a positive voltage difference and a negative voltage difference were applied to the experimental set up described in Example 1.

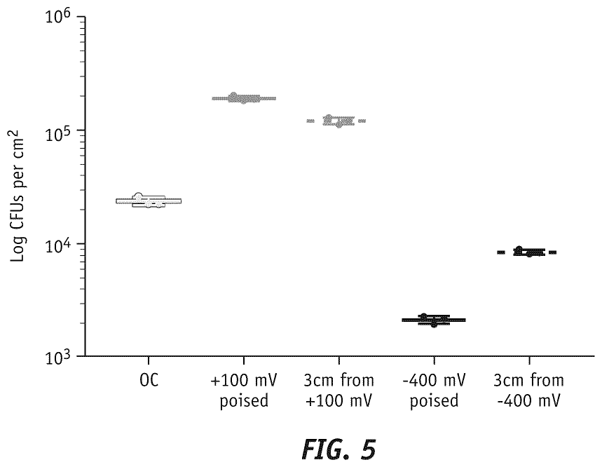

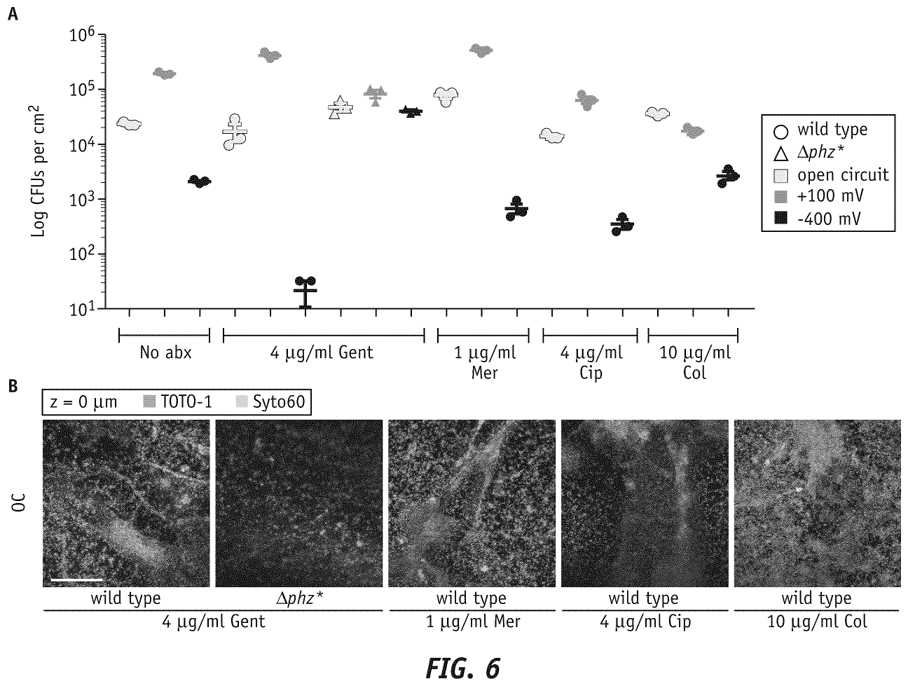

The results reported in FIG. 1C show that compared with OC control conditions, anoxic PYO-oxidative conditions enhanced cell survival by 10-fold while PYO-reductive conditions decreased wild-type (WT) cell survival by 10-fold (CFUs/cm² s standard error [SE],n=3, for OC=[2.35±0.11]×10⁴, +100 mV=[1.91±0.06]×105, and −400 mV=[2.10±0.09]×10³, FIG. 1C and FIG. 4]. This electrode potential-dependent effect on survivability was also observed for biofilms grown on glass surfaces placed −3 cm from the working electrode (FIG. 5), suggesting that redox cycling enabled by an electrode at a distance can support cell survival, as expected from previous studies in planktonic culture [31]). CFUs of phenazine-deficient Δphz* biofilms grown without PYO were not affected by the potential applied to the electrodes but were sensitized under PYO-reductive conditions by the addition of 10 μM PYO, indicating that cell survival in this context is PYO mediated (FIGS. 1A to D and FIG. 3).

Biofilm morphology was qualitatively consistent with results from CFU counts, with biofilms treated under PYO-oxidative conditions showing full electrode surface coverage and secondary structures up to 100 μm thick; large microcolonies stained brightly with SYTO 60 in the core yet took up TOTO-1 in the periphery (FIGS. 1D and 1E). As these dyes provide a measure of cell permeability as well as staining extracellular DNA (TOTO-1), consistent with previous studies ([49]), we interpret these results to indicate that cells in the interior were intact, whereas those on the periphery had compromised membranes.

In comparison, biofilms treated under PYO-reductive conditions were made up of single-cell layers with no secondary structures and a greater proportion of membrane-permeable cells (FIGS. 1D and 1E). This pattern held true for all samples. Addition of PYO to Δphz* biofilms did not fully recapitulate the WT morphology possibly due to a lower amount of extracellular DNA in the biofilm matrix; eDNA release has been shown to be stimulated by PYO production ([50]), and eDNA is also necessary for PYO retention ([33]).

### Example 3: Reduced PYO Acts Synergistically with Antibiotics to Kill Cells

In order to test the effect of antibiotic in combination with application of a difference in voltage to P. aenuginosa, sub-MICs of gentamicin, meropenem, ciprofloxacin, and colistin were added to anoxic survival reactors set up as described in Examples 1 and 2.

Most notably, the addition of 4 μg/mL of gentamicin to PYO-reductive conditions almost fully eradicated WT biofilms (CFU/cm² g SE, in =3, for OC=[1.71±0.59]×10⁴, −400 mV=21.4±10.7) but did not affect Δphz* biofilms. As PYO has been shown to confer tolerance to aminoglycosides ([51]), our data suggest that oxidized PYO confers tolerance to aminoglycosides, which is also disabled by PYO that is biochemically altered ([52]).

Alternatively, or in addition, our data are consistent with previous reports indicating reduced phenazines can be toxic in the presence of a sufficient concentration of iron in the medium to trigger the formation of phenazine radical species ([53]); assuming such reactions were also at play under our conditions, this mode of toxicity may have contributed to causing cell death when combined with aminoglycosides.

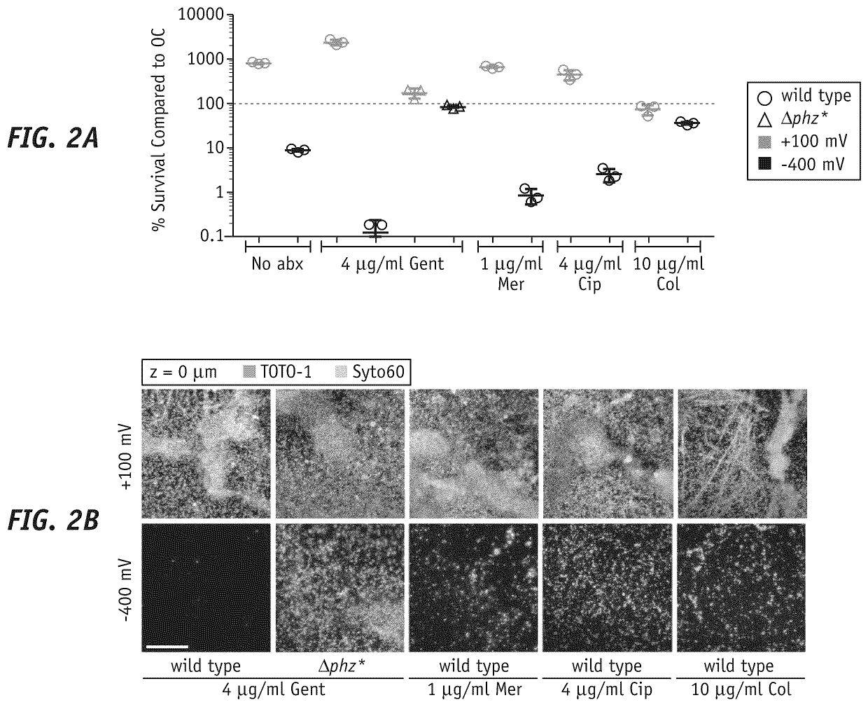

Treatment with 1 μg/mL meropenem (a β-lactam) or 4 μg/mL ciprofloxacin (a fluoroquinolone) under PYO-reductive conditions also decreased cell survival compared with OC by ˜100×(CFU/cm²±SE, in =3, for meropenem OC=[7.72±0.95]×10⁴, −400 mV=[6.73±1.47]×10²; for ciprofloxacin OC=[1.39±0.11]×10⁴, −400 mV=[3.53±0.66]×10²) but cell clusters sparsely covering the electrode surface were still present (FIG. 2B). Consistent with our results, previous reports have shown that PYO confers tolerance to ciprofloxacin and the β-lactam carbenicillin, with Δphz* cells in either colony biofilms or liquid cultures being more susceptible to ciprofloxacin and carbenicillin than wild type ([54], [50]).

Treatment with 10 μg/mL colistin did not show an additive effect with PYO-reductive conditions, yet PYO-oxidative conditions in the presence of colistin showed a 10× decrease in CFUs compared with nontreated biofilms (CFU/cm²±SE, in =3, +100 mV+10 μg/mL colistin=[1.74±0.15]×10⁴, +100 mV no antibiotic=[1.91±0.06]×105), a finding consistent with a previous study showing that colistin synergizes with phenazines to kill cells in colony biofilms under oxic conditions ([54]). Based on our results under anoxic conditions with cell-permeability dyes, it was hypothesized that PYO-oxidative conditions are most likely to mimic those within colony biofilms since a larger proportion of metabolically active cells arises when oxidized PYO is available (FIG. 2B), and this resembles what we would expect for cells within colony biofilms grown under oxic conditions ([55]).

### Example 4: Toxicity of Electrical Current on P aeruginosa in Biofilms and Cloture

To characterize possible toxic effects of reduced PYO on cell survival, biofilms were harvested pretransfer under oxic conditions and after 30 min, 6, 36, and 72 h after transfer to anoxic conditions. Plating was done in parallel on both oxic and anoxic media to rule out the effects of experimental setup on cell death. No significant difference was observed between CFUs of biofilms pretransfer (oxic) at −400 mV vs Ag/AgCl and +100 mV vs Ag/AgCl.

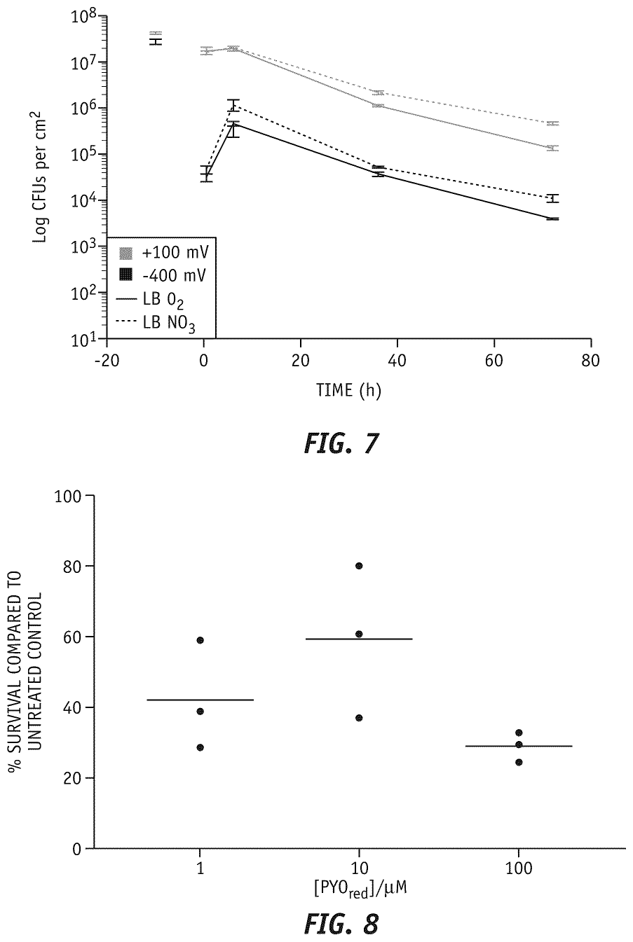

After 30 min from transferring to anoxic reactors, CFUs from biofilms grown under PYO-reductive conditions decreased 100-fold compared with original aerobic biofilms, while PYO-oxidative conditions only caused a slight decrease in CFUs (FIG. 7). While such rapid killing is consistent with PYO toxicity, under anoxic PYO-reductive conditions, EET is also disrupted in biofilms previously grown under oxic conditions.

Therefore, mid-log aerobic cell cultures were used to inoculate anoxic medium containing a biochemical O₂ scavenging system for which EET is not possible in the absence of both an electrode or O₂.

The results of the related experiments show that increasing concentrations of reduced PYO led to a two- to threefold decrease in CFUs (FIG. 8). However, increasing concentrations of reduced PYO did not correlate with a decrease in CFUs and did not achieve the 10-fold decrease seen in biofilm experiments between OC and PYO-reductive conditions.

The discrepancy between liquid culture and biofilm experiments is expected to be possibly due to (i) an antitoxicity pathway present in fresh liquid cultures but unexpressed in cells within week-old biofilms or (ii) the presence of a working electrode constantly driving the PYO pool toward a fully reduced state or creating secondary toxic products at the biofilm attachment surface. Alternatively, it is expected that EET disruption as being more critical for biofilm cell death than generation of reduced PYO per se.

Taken together, our results highlight the importance of redox cycling for P. aeruginosa survival within oxygen-limited biofilms and demonstrate that electrochemical manipulation, in tandem with antibiotic treatment, can be applied to better control biofilms of opportunistic pathogens.

As redox cycling both promotes EET and decreases the amount of reduced PYO in a P. aeruginosa biofilm. The results of the experiments provide context for the mechanism behind previous observations of cell death in the presence of a weak electric current ([56]) and provides conditions under which existing electrical bandage technology ([57]) can be modified to become more host compatible.

In particular, the results of the experiments reported in Examples 1 to 4 of the present disclosure support the conclusion that P. aeruginosa biofilms and can act synergistically with antibiotic treatment in particular the results reported in this experimental section support effectiveness a method of to decrease viability and kill cells in a biofilm by: (1) applying a reducing potential (−400 mV vs Ag/AgCl) to disrupt pyocyanin redox cycling by maintaining pyocyanin in the reduced state and (2) treatment of antibiotics, including a sub-MIC range of antibiotics. Through application of this method, a 100-fold decrease in CFUs within these biofilms compared to those exposed to electrodes poised at +100 mV vs Ag/AgCl, was observed.

Phenazine-deficient Δphz* biofilms were unaffected by the potential applied to the electrode, but were re-sensitized by adding pyocyanin. The effect at −400 mV was exacerbated when biofilms were treated with sub-MICs of a range of antibiotics. Most notably, addition of the aminoglycoside gentamicin in a reductive environment almost completely eradicated wild type biofilms but had no effect on the survival of Δphz* biofilms in the absence of phenazines. These data support the conclusion that antibiotic treatment combined with electrochemical disruption of pyocyanin redox cycling, either through the toxicity of accumulated reduced pyocyanin or the disruption of EET, or both, can lead to extensive killing.

Therefore, the data provided herein support the conclusion that electrochemically controlling the redox state of phenazines exemplified here by the representative pyocyanin, which enable the extracellular electron transfer (EET) within oxygen-limited regions can impact and in particular inhibit cell survival within anaerobic environment and in particular biofilms.

### Example 5: Timing Diagram

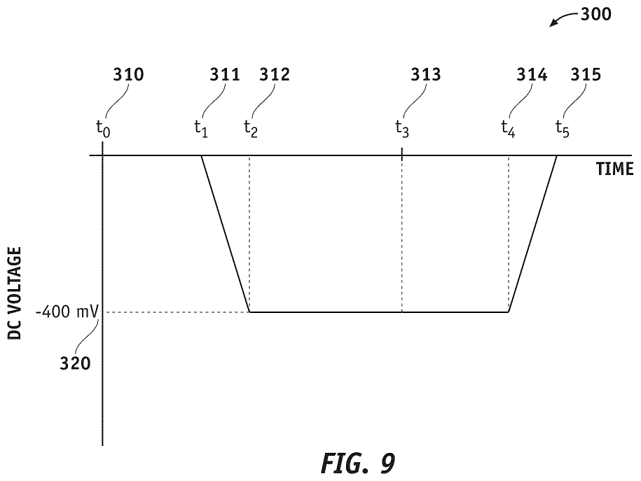

FIG. 9 shows an example timing diagram of the method to decrease viability of bacterial cells. The voltage of the x-axis represents the voltage across two electrodes where the voltage is given from the vantage point of the killing zone of the bacterial cells (e.g., at the working electrode) in respect to the other electrode (e.g., the counter electrode).

The two electrodes may be a cathode and an anode. At time t0 (310), there is no potential difference across the two electrodes. At time t1 (311), the voltage potential begins growing negatively. In other words, the voltage potential of the cathode becomes negative with respect to the anode, or the voltage potential of a first electrode becomes negative with respect to the anode.

At time t2 (312), the voltage potential difference between the two electrodes reaches its maximum (320)(i.e. 400 mV), with a first node (e.g., working electrode) being a negative potential with respect to a second node (e.g., counter electrode). This voltage (320) is the pyocyanin-reductive electrical potential. For example, the first node may be −400 mV with respect to the second node (i.e. 0V). The voltage holds constant between t2 (312) and t3 (313).

During this time, the pyocyanin is in a reduced state. At time t3 (313), the P. aeruginosa biofilm is treated with antibiotics. At time t4 (314), the antibiotics have been released into the biofilm, and the voltage difference between the first electrode and the second electrode begins to decrease (get closer to 0V potential between the electrodes). At time t5 (315), the voltage difference returns to 0V.

Given that the application of the potential makes the biofilm more susceptible to antibiotics, the potential must be held long enough so that the antibiotic can eradicate the biofilm. The specific potential depends upon the respiration potential of the biofilm.

The voltage (320) shown on FIG. 9 is an example of a negative voltage that can be applied prior to the antibiotic beings applied. Other voltages may be applied for this method. For example, the voltage across the electrodes may be as high as 600 mV or as low as 300 mV. Experiments have shown that 400 mV is the optimal voltage value to be applied (i.e., −400 mV at the working electrode).

It should be noted that it is possible to hold a lower voltage (closer to 0V) for a longer period of time in order to achieve similar results.

In some embodiments, the voltage is controlled by monitoring the applied voltage (e.g., reference electrode or external probe). In some embodiments, a look-up table can be used where known inputs result in the desired voltage based on already-tested application parameters (e.g., wound type, patient statistics, anti-bacterial agent type, etc.).

### Example 6: Bandage with Two Electrodes and Antibiotic Releasing Chip

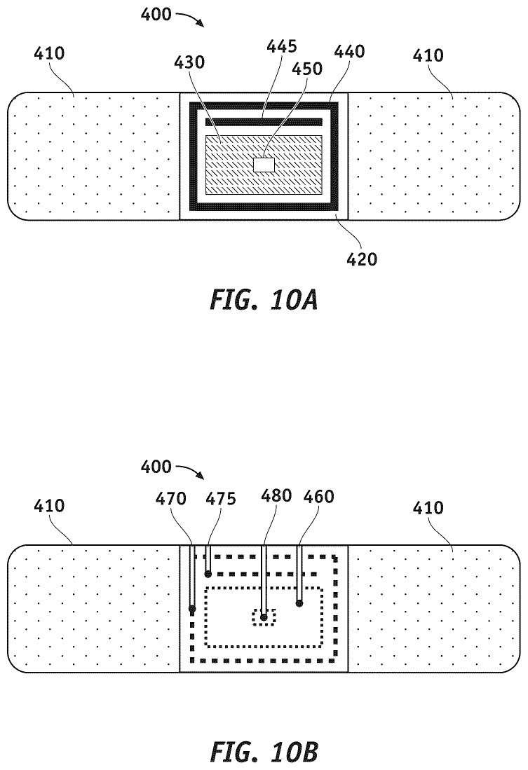

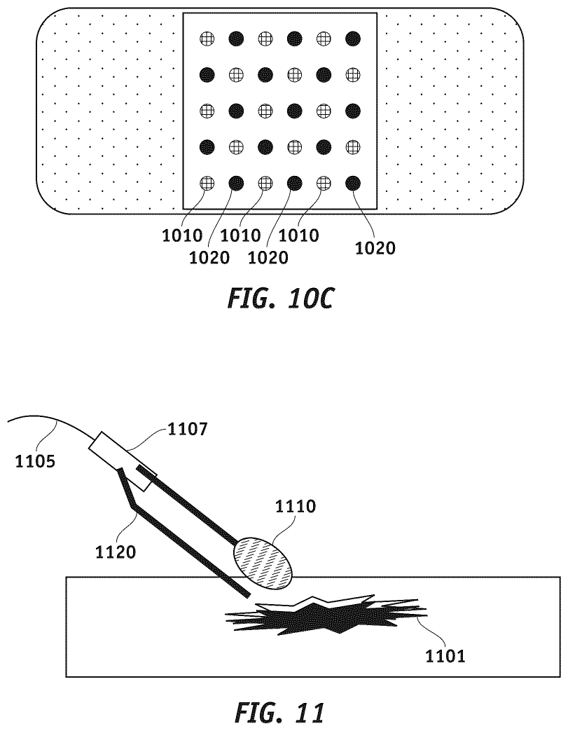

In some embodiments, a single bandage may be used to both deliver the pyocyanin-reductive electrical potential to the P. aeruginosa biofilm and to treat the P. aeruginosa biofilm with antibiotics. FIG. 10 and FIG. 10B show a bandage with this capability.

FIG. 10A shows the bottom side of the bandage to be in contact with a patient's open wound. The bandage contains sections (410) of adhesive used to adhere the bandage on to the position of interest. The center section (420) of the bandage contains both the working electrode (430) and the counter electrode (440) to apply the electrical potential, an optional third electrode (445), and antibiotic releasing chip (450) needed to treat the biofilm. The third electrode may operate as a reference electrode. The working electrode (430) is in contact with the biofilm and is energized at a negative potential compared to the counter electrode (440), which can be measured by the reference electrode (445).

This bandage may be charged by a wired connection, as shown in FIG. 10B, or wirelessly (not shown). When the bandage is placed on the patient in the position of interest, the electrodes will come in contact with the open wound. The antibiotic releasing chip may receive a signal to indicate that pyocyanin is in a reduced state. This signal may come after a certain predetermined period of time after the voltage is applied. The signal may be from a wired connection, as shown in FIG. 10B, or wirelessly (not shown).

FIG. 10B shows the top side of the bandage. In this embodiment, wired connections (460, 470) supply voltage to the bandage and a wired connection (480) provides a signal to the chip to release the antibiotic at a provided time. If there is a reference electrode, a further wired connection (475) allows reading the voltage. The timing of this signal aligns with t3 (313) from the timing diagram of FIG. 9.

In some embodiments, the bandage can include an array of electrodes. As example, FIG. 10C shows an array of working (1010) and counter (1020) electrodes arrayed in an alternating manner. One or more reference electrodes can also be incorporated, along with one or more anti-biotic delivery chips.

In some embodiments, the electrodes in the array of electrodes can include conductive needle structures to allow the electrodes to enter the skin of the patient.

In some embodiments, the array of electrodes can be incorporated into a fabric that can be used to sterilize any surface or item by covering a surface with the fabric (energized) and an anti-bacterial liquid or gel.

In some embodiments, the bandage is impregnated with anti-bacterial gel or liquid.

### Example 7 Electrosurgery

Electrosurgery is a method of applying a voltage within the body. An electrosurgical unit consists of a generator and a handpiece with one or more electrodes. The device is controlled using a switch on the handpiece or a foot switch. Two types of electrosurgery are described.

In one embodiment of an electrosurgery system (FIG. 11), both the active electrode and return electrode functions are performed at the site of surgery (1101) to sterilize a region being operated on. In an example, two tips of a powered (here with a wire (1105)) forceps-like device (1107) perform the working (1110) and counter (1120) electrode functions. Only the tissue grasped in the forceps is included in the electrical circuit. A anti-bacteria spray/application tube (1125) can also be attached to the forceps for easy application, or be separately provided.

In some embodiments for electrosurgery systems, the working electrode is a conductive paddle to cover a larger area. Various sizes and shapes of working electrodes can be utilized based on the expected size and shape of the area being sterilized. For example, a long probe can be used to enter deep incisions/wounds in the patient.

In some embodiments, variations of an electrosurgery system can be adapted to spot-sterilize equipment or surfaces by applying the electrodes to non-conductive surfaces that have had a liquid or gel (such as an anti-biotic) applied to it.

### Example 8 Orthopedic Device

Implant-associated bacterial infections are not uncommon. It is beneficial to be able to treat infections for orthopedic devices without performing any additional surgery or operations. Therefore, an embodiment of the disclosure includes the ability to generate and apply a voltage from the orthopedic device and the ability to release and thus administer an antibiotic from the orthopedic device.

To apply a voltage within the body near the site of the infection, the orthopedic device may be used as a voltage generating mechanism. The orthopedic device may have charge stored inside of it at the time of implant (in a battery). At a prescribed time (either by external control of the device, or at a designated pre-programmed time), the battery may activate, allowing a potential voltage to develop across part of the orthopedic. Many orthopedics are conductors, and a voltage applied across an orthopedic device will result in a current corresponding to the voltage applied and the resistance of the orthopedic device. Antibiotics may be released from within the orthopedic device itself, or may be administered separately from the orthopedic device (i.e. orally, intravenously, etc.). This voltage, in conjunction with the timed release of the antibiotics, can be an effective method to prevent, reduce, or eliminate a bacterial infection.

In some embodiments, an orthopedic device may be capable of releasing an antibiotic, if triggered, to attack a bacterial infection, or prevent a bacterial infection from occurring. Antibiotics may be released from holes or pockets in the orthopedic. The opening of these holes or pockets may be triggered from within the orthopedic, or externally. If the prescribed voltage is applied in conjunction with this release of antibiotic, the infection may be substantially minimized or completely eliminated or avoided.

Exemplary implants are shown Csernátony, et al 2021 [58], the implants and material described in Zoller et al 2020 [59], the implants and material described Xi et al. 2021 [60], and implants and material described in Park et al 2021 [61].

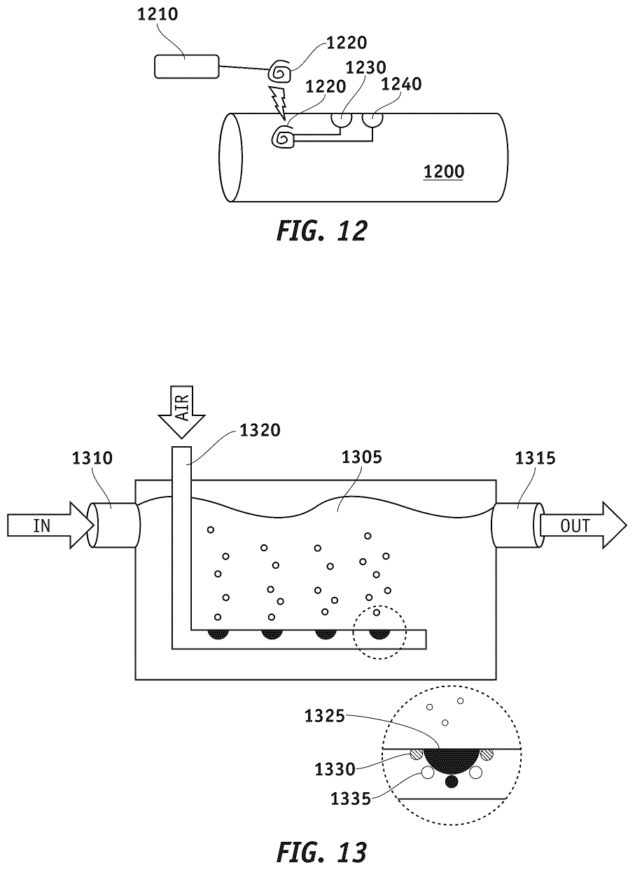

It is envisioned that implants such as the exemplary implants discussed in the present example can be modified to include a wireless battery connection and electrodes in a configuration directed to apply the voltage difference to the surfaces of the implants where redox bacteria are known or expected to be formed. An example is shown in FIG. 12, showing an example implant (1200) with an external battery/power source (1210), power relay coils (1220), and working electrodes (1230) and counter electrodes (1240) in an array configuration (only two shown in FIG. 12, but can include many). The related activation can occur. Note that if a metal implant is used, the metal of the implant will need to be insulated from at least one type of electrode (working and/or counter)

It is also envisioned that implants such as the exemplary implants discussed in the present example can be modified to include a reservoir for controlled release of antibiotic and/or antimicrobials such as the compartment described in the following Example 9 as will be understood by a skilled person upon reading of the present disclosure.

### Example 9: Controlled Antibiotic Release

Implant devices or other devices which can be configured to include a configuration of battery and electrode directed to apply a difference in voltage to a target portion of the devices and/or of surrounding tissues (if the devices are implantable or in contact with tissue of an individual) these devices can possibly be modified to include a compartment with controlled release of antibiotic.

An example is discussed in Gimeno et al 2015 [62] in the illustration of medical grade tubes modified to provide drug storage reservoirs and delivery implants with one open end (used to load the corresponding antibiotic) and a blind end welded on the opposite side. As indicated in Gimeno et al 2015 [62], four different models of implants were designed with a variable number of through pinholes (2, 4, 6 and 8 equidistant orifices), each with a diameter of 500 m. Each implant is 2.5 cm long and 0.6 cm O.D. having a wall thickness of 1.6 mm.

In the exemplary configuration of Gimeno et al 2015 [62] the antibiotics were loaded as a dry solid. And the mechanism of release relied on liquid from the outside entering the reservoir to dissolve the antibiotic before the dissolved molecules could diffuse out.

Additional configurations of the pinholes and additional formulations of antibiotics can be used for controlled release of antibiotics or other antimicrobials as will be understood by a skilled person.

### Example 10: Disinfecting a Device or an Object

This method of disinfecting an internal device can also be applied to other devices, specifically medical devices, such as a catheter or tubing. Applying a voltage outside of the body may be done either by the device itself (if the device has a battery or some amount of stored energy), or by externally apply voltage. Another method of disinfecting the device is to submerge the device in a solution to which the two- or three-electrode system previously described may be applied. The voltage applied to the area of interest sets the environment as previously described for antibiotic treatment. The antibiotic may be applied directly to the device, in, for example, a gel solution, liquid solution, or spray solution.

### Example 11 Aerator Disinfection

It is known that bacterial outbreaks have been traced to contaminated waters sources, specifically those with aerators. Using techniques described in this disclosure, it is expected to implement the voltage application and antibiotic treatment to treat the P. aeruginosa biofilm.

Provided that the biofilm is immersed in a liquid environment, a potential voltage may be applied, and then antibiotics may be applied to disinfect the aerator device.

An example aerator device is shown in FIG. 13. In an aerator tank (1305) with an input (1310), and output (1315), and aeration tube (1320), an anti-bacterial field can be created around the aeration holes (1325) by placing working electrodes (1330) and counter electrodes (1335) in an alternating array around the holes. An anti-bacterial agent can also be included in the tank (1305) or on the surface of the tube (1320) or as part of the input (1310).

### Example 12 Configurations of the Electrodes

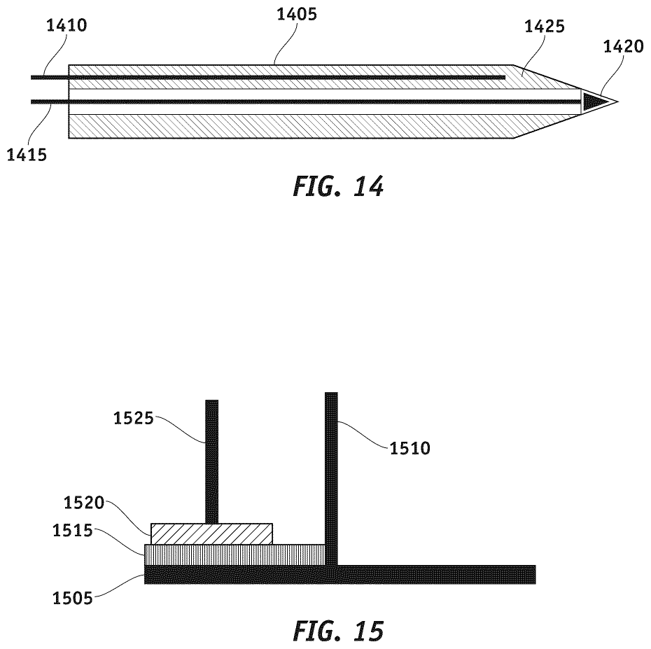

In some embodiments, the reference and counter electrodes can be combined into a single element. FIG. 14 shows an example combination reference and counter electrode in cross-sectional view. An outer casing (1405) holds the reference electrode (1410) and the counter electrode (1415). The counter electrode can end in a conductive tip (1420). The casing (1405) can also be conductive so long as there is resistance between the two electrodes (1410, 1415). The reference electrode can be held in a resistive layer (1425).

In some embodiments, the working electrode and the counter electrode can be combined into a single element. FIG. 15 shows an example combination working and counter electrode in cross-sectional view. The working electrode (1505) is connected to a power source through a line (1510). The counter electrode (1520) has its own connection to power (1525) and is separated from the working electrode by an insulating layer (1515). The working electrode (1505) can be an e-scaffold design as shown in Sultana et al 2015 [63].

### Example 13: Determination of the Midpoint Potential of a Redox-Active Molecule

A redox-active molecule can be isolated from a biological source and purified by methods known to a skilled person (see Wang and Newman, “Redox Reactions of Phenazine Antibiotics with Ferric (Hydr)oxides and Molecular Oxygen”, Environ. Sci. Technol. 2008, 42, 2380-2386).

The purified redox-active molecule can be dissolved in aqueous solution at appropriate concentration in the presence of supporting electrolyte and buffer to select the pH of measurement. For example, the redox-active molecule can be dissolved in 10 μM-10 mM concentrations in distilled, deionized water (DDW). The supporting electrolyte can be a salt such as NaCl or KCl at 0.01-0.5 M concentrations, for example, dissolved in DDW. The buffer can be 10 mM ammonium acetate/3-(N-morpholino)propanesulfonic acid at pH 7, or phosphate-buffered saline (PBS) at pH 7.4, or sodium acetate buffer at pH 5, or di-sodium hydrogen phosphate/potassium dihydrogen phosphate at pH 9, or boric acid/citric acid/trisodium phosphate for a wide range of pH values, for example.

Voltammetric methods are then used to characterize the electrochemical behavior of the redox-active molecule in such solution, with a potentiostat such as a Gamry PC4-300, using a three-electrode setup with working electrode (glassy carbon, or platinum, or gold), counter electrode (platinum wire) and reference electrode (Ag/AgCl (3M KCl), or SCE, or NHE; values are reported relative to Ag/AgCl (3M KCl) which has potential +197 mV vs. NHE at 298K).

For example, cyclic voltammetry may be used. In a cyclic voltammetry experiment phenazines will show a peak on the cathodic sweep (denoted E_{pc}) corresponding to the reduction of the redox molecule.

The reverse, anodic sweep will show a peak (denoted Eₚₐ) corresponding to the re-oxidation of the redox molecule. If the electrode reaction is diffusion-controlled and reversible, the cathodic and anodic peak currents will be equal in area, and the mid-point between these peaks may be taken as the formal reduction potential of the redox molecule under the experimental conditions used. This value can interchangeably be referred to as the “midpoint potential” or “mid-peak potential”, denoted E_{1/2}.

E_{1/2} values may be measured by a variety of experimental procedures as may be understood by the skilled person, including cyclic voltammetry, differential pulse voltammetry, square wave voltammetry, direct current polarography and redox titration; suitable methods are described in “Electrochemical Methods: Fundamentals and Applications” 3rd Ed., A. J. Bard, L. R. Faulkner and H. S. White.

### Example 14: Determination of the Midpoint Potential of Pyocyanin, PYO

PYO was purified from aerobic bacterial cultures and was dissolved in 0.1 M KCl aqueous solution buffered with 10 mM ammonium acetate-3-(N-morpholino)propanesulfonic acid (5 mM each, pH 7) to yield a stock solution concentration of 829 μM PYO. Electrochemical measurements were performed with a Gamry PC4-300 potentiostat. A stationary gold disk electrode (BASi) was used as the working electrode, an Ag/AgCl electrode (RE-51, BASi) as the reference, and a straight platinum wire (BASi) was the counter electrode. Scans were performed between 20 and 500 mV/s in the potential range of around E_{1/2} (+/−300 mV), at 298 K.

At pH 7, 298 K the E_{1/2} for PYO was measured to be −237 mV s. Ag/AgCl (3M KCl), or −40 mV vs. NHE (corrected to NHE by adding 197 mV to the value measured vs. Ag/AgCl).

It will be understood that experimental error considerations result in the E_{1/2} values quoted in a particular measurement to be accurate to within +/−20 mV between experiments.

### Example 15: Midpoint Potentials Reported for Redox Molecules of Interest

Representative E_{1/2} values for bacterially-produced redox molecules of interest, measured by the methods described are given in the Table below:

| | E_{1/2} (vs. Ag/AgCl | | | |
|---|---|---|---|---|
| | (3M KCl))/mV | | | |
| | pH 5 | pH 6 | pH 7 | pH 8 |
| 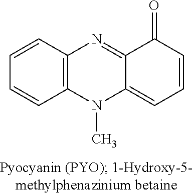 [SMILES: Cn1c2cccc(=O)c-2nc2ccccc21] | −128 | −189 | −237 | −300 |
| 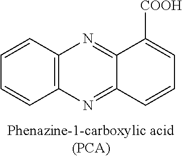 [SMILES: O=C(O)c1cccc2nc3ccccc3nc12] | −185 | −257 | −313 | |
| 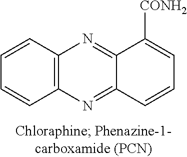 [SMILES: NC(=O)c1cccc2nc3ccccc3nc12] | −205 | −278 | −337 | |
| 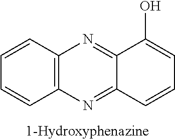 [SMILES: Oc1cccc2nc3ccccc3nc12] | −254 | −323 | −371 | −442 |
| 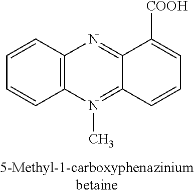 | | | 0 | |
| 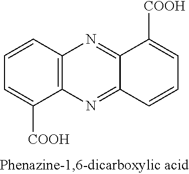 [SMILES: O=C(O)c1cccc2nc3c(C(=O)O)cccc3nc12] | | | 0 | |
| 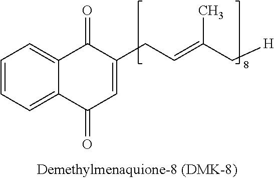 | | | −161 | |
| 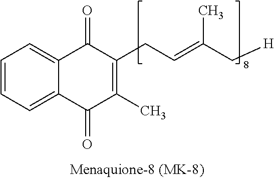 | | | −271 | |
| 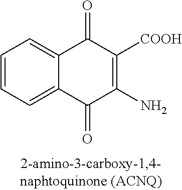 [SMILES: NC1=C(C(=O)O)C(=O)c2ccccc2C1=O] | | | −250 | |
| 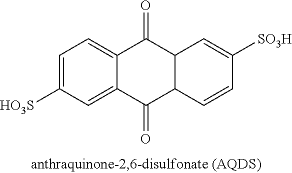 [SMILES: O=C1c2ccc(S(=O)(=O)O)cc2C(=O)C2C=CC(S(=O)(=O)O)=CC12] | | | −381 | |

### Example: Estimated Correction of Midpoint Potential Measured Under One Set of pH Conditions to Another Set of pH Conditions

As described in en.wikipedia.org/wiki/Nernst_equation, the Nernst equation relates E_{1/2} measured at pH 7 to the E_{1/2} at a different pH by:

$${E}_{1 / 2} = {E}_{1 / 2} ( pH 7 ) - 0.059 ( h / z ) Δ pH$$

where E_{1/2} is in V
h is the number of protons transferred in the electrochemical reaction
e is the number of electrons transferred in the electrochemical reaction

Pyocyanin undergoes a coupled 2 electron/2 proton redox process:

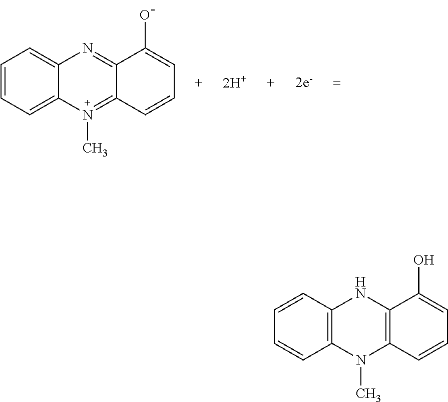

Hence, for example the E_{1/2} value measured for pyocyanin in the Table above at pH 7 (−237 mV vs. Ag/AgCl (3M KCl) at pH 7) may be estimated at pH 6 in the following way:
ΔpH=−1 (from pH 7 to pH 6)
h=2 (2 protons)
e=2 (2 electrons)

$${E}_{1 / 2} ( ph 6 ) = - 0.237 - 0.059 * ( 2 / 2 ) * - 1 = - 0.178 V = - 178 mV$$

Similarly, E_{1/2} for pyocyanin at pH 8 may be estimated to be −296 mV.

### Example 16: Selecting Practical Operating Potential to Apply for a Target Redox Active Compound

It will be understood that the midpoint potential, E_{1/2}, represents the potential whereby the reduced redox molecule is present in equal concentration with its oxidized form. Therefore, to drive the reaction further to reduce the vast majority of the redox molecules in the vicinity of the working electrode, the applied potential needs to be lower than the E_{1/2} value measured or reported for a particular set of conditions (e.g., pH); for example, applying a potential 100-150 mV below the E_{1/2} value can result in more than 98% of the redox molecule being reduced. Similarly, applying a potential 200 mV or more below the E_{1/2} value can result in approximately 100% reduction of the redox molecule.

It will also be understood that the medium itself has a limit at which increasingly lower potential will cause its decomposition. For example, hydrogen is generated by the reduction of water at low potentials, and the by-products of hydrogen evolution may impede wound healing or damage tissue, so operating below this potential is not recommended. This “voltage limit” or “voltage window” for a given medium varies as a function of conditions (for example, pH). The Pourbaix diagram for water is shown in FIG. 16, with the reference voltage values given relative to the NHE.

As is evident, this lower potential limit becomes more negative with increasing pH, varying as described by the Nernst equation above (1 electron, 1 proton reaction for hydrogen evolution).

This lower voltage limit is given vs. Ag/AgCl (3M KCl) for operating pH ranges of interest for the invention in the Table below:

| | pH | | | | |
|---|---|---|---|---|---|
| | 5 | 6 | 7 | 8 | 9 |
| lower limit vs. Ag/AgCl | −492 | −551 | −610 | −669 | −728 |
| (3M KCl)/mV | | | | | |

Therefore, care is taken not to exceed this lower voltage limit when selecting what potential to apply for a given redox molecule.

For example, for pyocyanin in an environment at pH 6, the E_{1/2} value may be estimated to be −178 mV vs. Ag/AgCl (3M KCl); therefore, an operating potential from −278 mV to −500 mV may usefully be applied to satisfy both a) reducing close to 100% of the pyocyanin present and inhibiting its re-oxidation and b) operating within the stability limit of the local environment medium.

For a second example, for 1-hydroxyphenazine, the measured E_{1/2} at pH 8 is −442 mV vs. Ag/AgCl (3M KCl); therefore, an operating potential of −552 mV to −650 mV may be chosen.

In summary described herein are methods and systems and related devices and compositions for electrochemical control of viability of redox active bacteria. The electrochemical control is performed by applying to a working electrode contacting a medium known or suspected to comprise the redox active bacteria, a reducing potential which is lower of the midpoint potential of a redox active compound produced by the redox active bacteria.

The examples set forth above are provided to give those of ordinary skill in the art a complete disclosure and description of how to make and use the embodiments of the compounds, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically described by preferred embodiments, exemplary embodiments and optional features, modification and variation of the concepts herein disclosed can be resorted to by those skilled in the art upon the reading of the present disclosure, and that such modifications and variations are considered to be within the scope of this disclosure as defined by the appended claims.

In particular this disclosure reference us made to a number of terms, which shall be defined to have the following meanings unless otherwise specified:

The term “alkyl” as used herein refers to a linear, branched, or cyclic saturated hydrocarbon group typically although not necessarily containing 1 to about 15 carbon atoms, or 1 to about 6 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 15 carbon atoms. The term “cycloalkyl” intends a cyclic alkyl group, typically having 4 to 8, or 5 to 7, carbon atoms.

The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The terms “alkenyl” and “alkylene” refers to an alkenediyl group which is a divalent unsaturated aliphatic group, with two carbon atoms as points of attachment, a linear or branched, cyclo, cyclic or acyclic structure, at least one nonaromatic carbon-carbon double bond. Exemplary alkylenes includes 2-butene-1,4-diyl group (—CH₂CH═CHCH₂—). The term “alkenyl” used herein also encompasses oligomers of isoprene units [—CH₂CH═C(CH₃)CH₂-]ₙ-H) where n=1-20, most preferably n=5-13.

The terms “alkynyl” and “alkynylene” refers to an alkynediyl group which is a divalent unsaturated aliphatic group, with two carbon atoms as points of attachment, a linear or branched, cyclo, cyclic or acyclic structure, at least one nonaromatic carbon-carbon triple bond. Exemplary alkylene includes 2-butyne-1,4-diyl group (—CH2C≡CCH2-).

The term “heteroatom-containing” as in a “heteroatom-containing alky group” refers to an alkyl group in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, and additional substituents identifiable by a skilled person.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Aryl groups can contain 5 to 24 carbon atoms, or contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail infra.

The terms “cyclic”, “cyclo-”, and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic.

The term “isomers” as used refers to heterocyclic aromatic groups that have the same core molecular but may differ in atomic connectivity and/or location of unsaturation and is meant to include all possible structural variants. For example, as shown below, “pyrrole isomers” refers to all possible substituted variants of 1H-pyrrole and 2H-pyrrole; “indole isomers” refers to all possible substituted variants of 3H-indole, 1H-indole and 2H-isoindole, and so on:

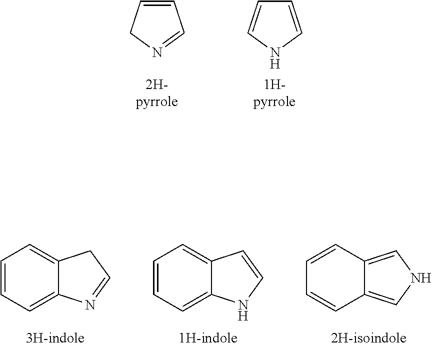 [SMILES: C1=CCN=C1 ; c1cc[nH]c1 ; C1=Nc2ccccc2C1 ; c1ccc2[nH]ccc2c1 ; c1ccc2c[nH]cc2c1]

Likewise, as shown below, “triazole isomers” refers to all possible substituted variants of 1,2,4-triazole and 1,2,3-triazole; and so on:

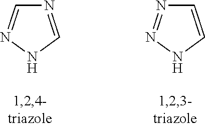 [SMILES: c1nc[nH]n1 ; c1c[nH]nn1]

The term “substituted” as in “substituted alkyl,” “substituted aryl,” and the like, is meant that in the alkyl, aryl, or other moiety, at least one hydrogen atom bound to a carbon (or other) atom is replaced with one or more non-hydrogen substituents.

Examples of such substituents include, without limitation: functional groups such as halo, hydroxyl, sulfhydryl, C1-C24 alkoxy, C2-C24 alkenyloxy, C2-C24 alkynyloxy, C5-C24 aryloxy, C6-C24 aralkyloxy, C6-C24 alkaryloxy, acyl (including C2-C24 alkylcarbonyl (—CO-alkyl) and C6-C24 arylcarbonyl (—CO-aryl)), acyloxy (—O-acyl, including C2-C24 alkylcarbonyloxy (—O—CO— alkyl) and C6-C24 arylcarbonyloxy (—O—CO-aryl)), C2-C24 alkoxycarbonyl (—(CO)—O-alkyl), C6-C24 aryloxycarbonyl (—(CO)—O-aryl), halocarbonyl (—CO)—X where X is halo), C2-C24 alkylcarbonato (—O—(CO)—O-alkyl), C6-C24 arylcarbonato (—O—(CO)—O-aryl), carboxy (—COOH), carboxylato (COO˜), carbamoyl (—(CO)—NH2), mono-(C1-C24 alkyl)-substituted carbamoyl (—(CO)—NH(C1-C24 alkyl)), di-(C1-C24 alkyl)-substituted carbamoyl (—(CO)—N(C1-C24 alkyl)2), mono-(C5-C24 aryl)-substituted carbamoyl (—(CO)—NH-aryl), di-(C5-C24 aryl)-substituted carbamoyl (—(CO)—N(C5-C24 aryl)2), di-N—(C1-C24 alkyl), N—(C5-C24 aryl)-substituted carbamoyl, thiocarbamoyl (—(CS)—NH2), mono-(C1-C24 alkyl)-substituted thiocarbamoyl (—(CO)—NH(C1-C24 alkyl)), di-(C1-C24 alkyl)-substituted thiocarbamoyl (—(CO)—N(C1-C24 alkyl)2), mono-(C5-C24 aryl)-substituted thiocarbamoyl (—(CO)—NH-aryl), di-(C5-C24 aryl)-substituted thiocarbamoyl (—(CO)—N(C5-C24 aryl)2), di-N—(C1-C24 alkyl), N—(C5-C24 aryl)-substituted thiocarbamoyl, carbamido (—NH—(CO)—NH2), cyano(-C≡N), cyanato (—O—C≡N), thiocyanato (—S—C≡N), formyl (—(CO)—H), thioformyl ((CS)—H), amino (—NH2), mono-(C1-C24 alkyl)-substituted amino, di-(C1-C24 alkyl)-substituted amino, mono-(C5-C24 aryl)-substituted amino, di-(C5-C24 aryl)-substituted amino, C2-C24 alkylamido (—NH—(CO)-alkyl), C6-C24 arylamido (—NH—(CO)-aryl), imino (—CR═NH where R=hydrogen, C1-C24 alkyl, C5-C24 aryl, C6-C24 alkaryl, C6-C24 aralkyl, etc.), C2-C20 alkylimino (CR═N(alkyl), where R=hydrogen, C1-C24 alkyl, C5-C24 aryl, C6-C24 alkaryl, C6-C24 aralkyl, etc.), arylimino (—CR═N(aryl), where R=hydrogen, C1-C20 alkyl, C5-C24 aryl, C6-C24 alkaryl, C6-C24 aralkyl, etc.), nitro (—NO2), nitroso (—NO), sulfo (—SO2-OH), sulfonato (—SO2-O⁻), C1-C24 alkylsulfanyl (—S-alkyl; also termed “alkylthio”), C5-C24 arylsulfanyl (—S-aryl; also termed “arylthio”), C1-C24 alkylsulfinyl (—(SO)-alkyl), C5-C24 arylsulfinyl (—(SO)-aryl), C1-C24 alkylsulfonyl (—SO2-alkyl), C5-C24 arylsulfonyl (—SO2-aryl), boryl (—BH2), borono (—B(OH)2), boronato (—B(OR)2 where R is alkyl or other hydrocarbyl), phosphono (—P(O)(OH)2), phosphonato (—P(O)(O⁻)2), phosphinato (—P(O)(O—)), phospho (—PO2), phosphino (—PH2), silyl (—SiR3 wherein R is hydrogen or hydrocarbyl), and silyloxy (—O-silyl); and the hydrocarbyl moieties C1-C24 alkyl (e.g. C1-C12 alkyl and C1-C6 alkyl), C2-C24 alkenyl (e.g. C2-C12 alkenyl and C2-C6 alkenyl), C2-C24 alkynyl (e.g. C2-C12 alkynyl and C2-C6 alkynyl), C5-C24 aryl (e.g. C5-C14 aryl), C6-C24 alkaryl (e.g. C6-C16 alkaryl), and C6-C24 aralkyl (e.g. C6-C16 aralkyl).

The term “acyl” refers to substituents having the formula —(CO)-alkyl, —(CO)-substituted alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “substituted alkyl”, “aryl, and “aralkyl” are as defined above.

The term “alkaryl” refers to an aryl group with an alkyl substituent, and the term “aralkyl” refers to an alkyl group with an aryl substituent, wherein “aryl” and “alkyl” are as defined above. In some embodiments, alkaryl and aralkyl groups contain 6 to 24 carbon atoms, and particularly alkaryl and aralkyl groups contain 6 to 16 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “Periodic Table” refers to the version of IUPAC Periodic Table of the Elements dated Nov. 28, 2016

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all sub-ranges, as well as erall individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which are not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

1. Pourbaix, W. Pourbaix Diagram. 2024 Available from: https://en.wikipedia.org/wiki/Pourbaix_diagram.
2. Hernandez, M. and D. Newman, Extracellular electron transfer. Cellular and Molecular Life Sciences CMLS, 2001. 58: p. 1562-1571.
3. Franza, T. and P. Gaudu, Quinones: more than electron shuttles. Research in Microbiology, 2022. 173(6-7): p. 103953.
4. Rosenbaum, F. P. and V. Müller, Energy conservation under extreme energy limitation: the role of cytochromes and quinones in acetogenic bacteria. Extremophiles, 2021. 25(5): p. 413-424.
5. Dahlem Junior, M. A., et al., Quinones as an efficient molecular scaffold in the antibacterial/antifungal or antitumoral arsenal. International Journal of Molecular Sciences, 2022. 23(22): p. 14108.
6. Van Beilen, J. W. and K. J. Hellingwerf, All three endogenous quinone species of Escherichia coli are involved in controlling the activity of the aerobic anaerobic response regulator ArcA. Frontiers in microbiology, 2016. 7: p. 208925.
7. Mentel, M., et al., Of two make one: the biosynthesis of phenazines. ChemBioChem, 2009. 10(14): p. 2295-2304.
8. Pierson, L. S. and E. A. Pierson, Metabolism and function of phenazines in bacteria: impacts on the behavior of bacteria in the environment and biotechnological processes. Applied microbiology and biotechnology, 2010. 86(6): p. 1659-1670.
9. Turner, J. M. and A. J. Messenger, Occurrence, biochemistry and physiology of phenazine pigment production. Advances in microbial physiology, 1986. 27: p. 211-275.
10. Skerman, V., V. McGowan, and P. Sneath, Approved lists of bacterial names (amended). 1989.
11. Dar, D., et al., Global landscape of phenazine biosynthesis and biodegradation reveals species-specific colonization patterns in agricultural soils and crop microbiomes. Elife, 2020. 9: p. e59726.
12. Chen, K., et al., Metabolic degradation of phenazine-1-carboxylic acid by the strain Sphingomonas sp. DP58: the identification of two metabolites. Biodegradation, 2008. 19(5): p. 659-667.
13. Wang, Y. and D. K. Newman, Redox reactions of phenazine antibiotics with ferric (hydr)oxides and molecular oxygen. Environmental Science & Technology, 2008. 42(7): p. 2380-2386.
14. Wang, Y. and D. K. Newman, Redox reactions of phenazine antibiotics withferric (hydr) oxides and molecular oxygen. Environmental science & technology, 2008. 42(7): p. 2380-2386.
15. Fultz, M. L. and R. A. Durst, Mediator compounds for the electrochemical study of biologicalredox svstems: a compilation. Analytica Chimica Acta, 1982. 140(1): p. 1-18.
16. Hernandez, M. E. and D. K. Newman, Extracellular electron transfer. Cellular and Molecular Life Sciences, 2001. 58(11): p. 1562-1571.
17. Hernandez, M. and D. Newman, Extracellular electron transfer. Cellular and Molecular Life Sciences CMLS, 2001. 58(11): p. 1562-1571.
18. Michaelis, L. and E. S. Hill, POTENTIOMETRIC STUDIES ON SEMIQUINONES. Journal of the American Chemical Society, 1933. 55: p. 1481-1494.
19. Michaelis, L. and E. S. Hill, THE VIOLOGEN INDICATORS. THE JOURNAL OF GENERAL PHYSIOLOGY, 1933. 16: p. 859-873.
20. Michaelis, L. and E. S. Hill, The viologen indicators. Journal of General Physiology, 1933. 16(6): p. 859-873.
21. Glasser, N. R., S. H. Saunders, and D. K. Newman, The colorful world of extracellular electron shuttles. Annual review of microbiology, 2017. 71: p. 731-751.
22. Sullivan, N. L., et al., Quantifying the dynamics of bacterial secondary metabolites by spectral multiphoton microscopy. ACS chemical biology, 2011. 6(9): p. 893-899.
23. Ciemniecki, J. A. and D. K. Newman, NADH dehydrogenases are the predominant phenazine reductases in the electron transport chain of Pseudomonas aeruginosa. Molecular Microbiology, 2023. 119(5): p. 560-573.
24. Möker, N., C. R. Dean, and J. Tao, Pseudomonas aeruginosa increases formation of multidrug-tolerant persister cells in response to quorum-sensing signaling molecules. Journal of bacteriology, 2010. 192(7): p. 1946-1955.
25. Heydorn, A., et al., Quantification of biofilm structures by the novel computer program COMSTAT. Microbiology, 2000. 146(10): p. 2395-2407.
26. Fisher, R. A., B. Gollan, and S. Helaine, Persistent bacterial infections and persister cells. Nature Reviews Microbiology, 2017. 15(8): p. 453-464.
27. Keogh, D., et al., Extracellular electron transfer powers Enterococcus faecalis biofilm metabolism. MBio, 2018. 9(2): p. 10.1128/mbio. 00626-17.
28. Light, S. H., et al., A flavin-based extracellular electron transfer mechanism in diverse Gram-positive bacteria. Nature, 2018. 562(7725): p. 140-144.
29. Friedheim, E. A., Pyocyanine, an accessory respiratory enzyme. The Journal of experimental medicine, 1931. 54(2): p. 207.
30. Schroth, M. N., et al., Epidemiology of Pseudomonas aeruginosa in agricultural areas. Journal of Medical Microbiology, 2018. 67(8): p. 1191-1201.
31. Wang, Y., S. E. Kern, and D. K. Newman, Endogenous phenazine antibiotics promote anaerobic survival of Pseudomonas aeruginosa via extracellular electron transfer. Journal of bacteriology, 2010. 192(1): p. 365-369.
32. Glasser, N. R., S. E. Kern, and D. K. Newman, Phenazine redox cycling enhances anaerobic survival in P seudomonas aeruginosa by facilitating generation of ATP and a proton-motiveforce. Molecular microbiology, 2014. 92(2): p. 399-412.
33. Saunders, S. H., et al., Extracellular DNA promotes efficient extracellular electron transfer by pyocyanin in Pseudomonas aeruginosa biofilms. Cell, 2020. 182(4): p. 919-932. e19.
34. Bard, A. J., L. R. Faulkner, and H. S. White, Electrochemical methods: fundamentals and applications. 2022: John Wiley & Sons.
35. Standard, W. 2024; Available from: en.wikipedia.org/wiki/Standard_electrode_potential_(data_page) at the filing date of the present disclosure.
36. Orenstein, D. M., Cystic fibrosis: A guide for patient and family 4th ed. 4th ed. 2011: Lippincott Williams & Wilkins.
37. The Annual UK Drug Tariff in The Stationary Office. 1988 (November), 1998 (May), 2007 (February) The UK Drug Tariff: London, UK.
38. Lazarus, G. S., et al., Definitions and guidelines for assessment of wounds and evaluation of healing. Arch Dermatol, 1994. 130(4): p. 489-493.
39. Boateng, J. S., et al., Wound healing dressings and drug delivery systems: a review. Journal of pharmaceutical sciences, 2008. 97(8): p. 2892-2923.
40. Gilliland, E. L., et al., Bacterial colonisation of leg ulcers and its effect on the success rate of skin grafting. Annals of the Royal College of Surgeons of England, 1988. 70(2): p. 105-108.
41. Armstrong, S. and C. Ruckley, Use of a fibrous dressing in exuding leg ulcers. Journal of Wound Care, 1997. 6(7): p. 322-324.
42. Hareendran, A., et al., Measuring the impact of venous leg ulcers on quality of life. Journal of Wound Care, 2005. 14(2): p. 53-57.
43. Naradzay, F. and R. Alson, Burns, thermal. Web MD, 2005.
44. Bolton, L. and L. Van Rijswijk, Wound dressings: meeting clinical and biological needs. Dermatology nursing, 1991. 3(3): p. 146-161.
45. Krasner, D., et al., The ABCs of wound care dressings. wound management, 1993. 66: p. 68-69.
46. Atlas, R. M., Handbook of microbiological media. 2004: CRC press.
47. Bosire, E. M. and M. A. Rosenbaum, Electrochemical potential influences phenazine production, electron transfer and consequently electric current generation by Pseudomonas aeruginosa. Frontiers in Microbiology, 2017. 8: p. 260405.
48. Bockris, J. M. and L. Oldfield, The oxidation-reduction reactions of hydrogen peroxide at inert metal electrodes and mercury cathodes. Transactions of the faraday society, 1955. 51: p. 249-259.
49. Okshevsky, M. and R. L. Meyer, Evaluation of fluorescent stains for visualizing extracellular DNA in biofilms. Journal of Microbiological Methods, 2014. 105: p. 102-104.
50. Meirelles, L. A. and D. K. Newman, Both toxic and beneficial effects of pyocyanin contribute to the lifecycle of Pseudomonas aeruginosa. Molecular microbiology, 2018. 110(6): p. 995-1010.
51. Schiessl, K. T., et al., Phenazine production promotes antibiotic tolerance and metabolic heterogeneity in Pseudomonas aeruginosa biofilms. Nature communications, 2019. 10(1): p. 762.
52. VanDrisse, C. M., et al., Computationally designed pyocyanin demethylase acts synergistically with tobramycin to kill recalcitrant Pseudomonas aeruginosa biofilms. Proceedings of the National Academy of Sciences, 2021. 118(12): p. e2022012118.
53. Kang, J., Y.-H. Cho, and Y. Lee, Pyocyanin and 1-Hydroxyphenazine promote anaerobic killing of Pseudomonas aeruginosa via single-electron transfer with ferrous iron. Microbiology Spectrum, 2022. 10(6): p. e02312-22.
54. Schiessl, K. T., et al., Phenazine production promotes antibiotic tolerance and metabolic heterogeneity in Pseudomonas aeruginosa biofilms. Nature communications, 2019. 10(1): p. 1-10.
55. Dietrich, L. E., et al., Bacterial community morphogenesis is intimately linked to the intracellular redox state. Journal of bacteriology, 2013. 195(7): p. 1371-1380.
56. Niepa, T. H., J. L. Gilbert, and D. Ren, Controlling Pseudomonas aeruginosa persister cells by weak electrochemical currents and synergistic effects with tobramycin. Biomaterials, 2012. 33(30): p. 7356-7365.
57. Raval, Y. S., et al., Hydrogen peroxide-generating electrochemical scaffold activity against trispecies biofilms. Antimicrobial agents and chemotherapy, 2020. 64(4): p. 10.1128/aac. 02332-19.
58. Csernátony, Z., et al., Metal implants and MRI: A mythbuster study. Glob. Imaging Insights, 2021. 6: p. 1-4.
59. Zoller, S. D., et al., Evading the host response: Staphylococcus “hiding” in cortical bone canalicular system causes increased bacterial burden. Bone Research, 2020. 8(1): p. 43.
60. Xi, W., et al., Point-of-care antimicrobial coating protects orthopaedic implants from bacterial challenge. Nature communications, 2021. 12(1): p. 5473.
61. Park, H. Y., et al., Comparison of two fluorescent probes in preclinical non-invasive imaging and image-guided debridement surgery of Staphylococcal biofilm implant infections. Scientific reports, 2021. 11(1): p. 1622.
62. Gimeno, M., et al., A controlled antibiotic release system to prevent orthopedic-implant associated infections: An in vitro study. European Journal of Pharmaceutics and Biopharmaceutics, 2015. 96: p. 264-271.
63. Sultana, S. T., et al., Electrochemical scaffold generates localized, low concentration of hydrogen peroxide that inhibits bacterial pathogens and biofilms. Scientific reports, 2015. 5(1): p. 1-10.

1. A method to decrease viability of redox active bacteria in a medium, the redox active bacteria producing a redox active compound having an oxidized state and a reduced state, the redox active compound further having a midpoint potential, the method comprising:
contacting the medium with
a working electrode having a working electrode potential compared to a reference electrode and
a counter electrode having a counter electrode potential compared to the reference electrode;
operating a voltage source to apply to the working electrode and the counter electrode a reducing voltage selected so that the working electrode potential has a reducing potential, lower than the midpoint potential of the redox active compound,
the operating performed for a time and under conditions to increase the concentration of the redox active compound in the reduced state in the medium, thus inhibiting viability of the redox active bacteria.

2. The method of claim 1, wherein the method further comprises selecting a voltage between the working electrode and counter electrode based on the midpoint potential of the redox active compound so that the working electrode potential is lower than the midpoint potential of the redox active compound, thus providing the reducing voltage.

3. The method of claim 1, wherein, the reducing potential of the working electrode is from 50 mV lower than the E_{1/2} potential to 250 mV lower than the E_{1/2} of the target redox compound.

4. The method of claim 1, wherein E_{1/2} of one or more target redox active compounds is between +138 mV to −517 mV vs Ag/AgCl at pH 7, and the reducing potential of the working electrode can range from −50 mV to −600 mV, preferably from −100 mV to −550 mV at pH 7.

5. The method of claim 1, wherein E_{1/2} of one or more target redox active compounds is between 0 mV to −500 mV vs Ag/AgCl at pH 7, and the reducing potential of the working electrode ranges from −50 mV to −600 mV, at pH 7.

6-35. (canceled)

36. The method of claim 1, wherein the redox active compound has formula (I) 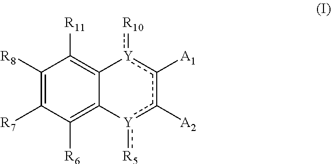
in which Y is C or N,
wherein when Y is C,
R₅ and R₁₀ are O and
A₁ is an alkyl or substituted alkyl, alkenyl, alkynyl, aryl, heteroaryl, acyl group, or COOH
A₂ is H or an alkyl or substituted alkyl, alkenyl, alkynyl, aryl, heteroaryl, acyl group or NH2, and
R₆, R₇, R₈ and R₉ are H,
wherein when Y is N
R₅ and R₁₀ are independently null, H, an alkyl group, an acyl group, or O
R₆, R₇, R₈ and R₉ are independently H, O, OH, COOH, C(O)H, NH₂, SH, Cl, Br, SO₃H, alkoxy, OC(O)-alkyl, C(O)O-alkyl, C(O)NH-alkyl, C(O)N-(alkyl)₂, S-alkyl, or an alkyl, substituted alkyl, alkenyl, alkynyl, aryl, heteroaryl or acyl group
and A₁, and A₂ are joined together to form a moiety of structure 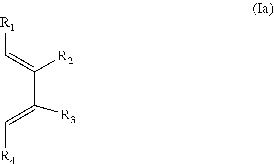
in which R₁, R₂, R₃, R₄ are independently H, O, OH, COOH, C(O)H, NH₂, SH, Cl, Br, SO₃H, alkoxy, OC(O)-alkyl, C(O)O-alkyl, C(O)NH-alkyl, C(O)N-(alkyl)2, S-alkyl, or an alkyl, substituted alkyl, alkenyl, alkynyl, aryl, heteroaryl or acyl group
and wherein the compound has a midpoint potential E_{1/2} such that +200 mV≤E₁/2> to −600 mV vs Ag/AgCl in an aqueous environment at pH7.

37. The method of claim 36, wherein the compound of Formula (I) has a midpoint potential E_{1/2} ranging from +138 mV to −517 mV vs Ag/AgCl in an aqueous environment at pH7.

38. The method of claim 36, wherein the compound of Formula (I) has a midpoint potential 0 mV s E₁/2> to −600 mV vs Ag/AgCl in an aqueous environment at pH7.

39. The method of claim 36, wherein the compound of Formula (I) has a midpoint potential E_{1/2} ranging from 0 mV to −517 mV vs Ag/AgCl in an aqueous environment at pH7.

40. The method of claim 36, wherein the compound of Formula (I) has a midpoint potential E_{1/2} ranging from 0 mV to −500 mV vs Ag/AgCl in an aqueous environment at pH7.

41. The method of claim 36, wherein the compound of Formula (I) has a midpoint potential E_{1/2} ranging from 0 mV to −400 mV vs Ag/AgCl in an aqueous environment at pH7.

42. The method of claim 36, wherein the compound of Formula (I) has a midpoint potential E_{1/2} ranging from −100 mV to −400 mV vs Ag/AgCl in an aqueous environment at pH7.

43. The method of claim 36, wherein the compound of Formula (I) has a midpoint potential E_{1/2} ranging from −125 mV to −375 mV vs Ag/AgCl in an aqueous environment at pH7.

44. The method of claim 36, wherein the compound of Formula (I) is selected from

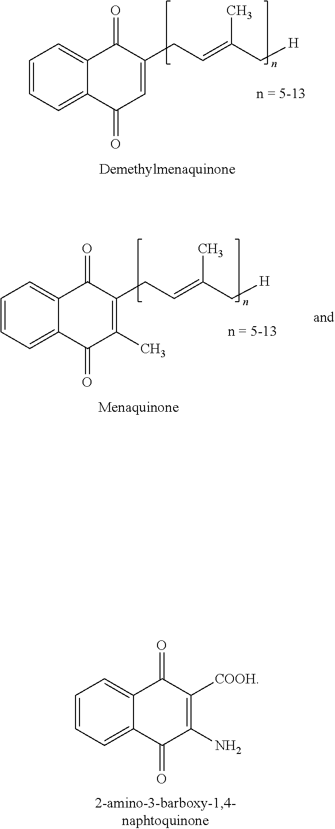

45. The method of claim 36, wherein the compound has Formula (II): 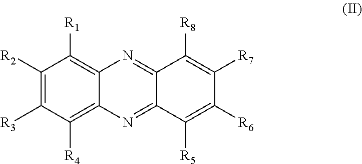
where R₁—R₈ are independently selected from hydrogen, hydroxy, alkoxy, alkyl, alkenyl, alkynyl, aryl, heteroaryl, and acyl.

46. The method of claim 36, wherein the compound has formula (III) 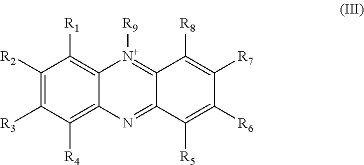
where R₁—R₁₀ are independently selected from hydrogen, hydroxy, alkoxy, alkyl, alkenyl, alkynyl, aryl, heteroaryl, and acyl, and one of R₁—R₁₀ is a negatively charged substituent (formal charge of −1).

47. The method of claim 36, wherein the compound is selected from the following structure 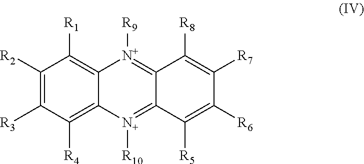
wherein R₁—R₁₀ are independently selected from hydrogen, hydroxy, alkoxy, alkyl, alkenyl, alkynyl, aryl, heteroaryl, acyl, and one of R₁—R₁₀ is a negatively charged substituent.

48. The method of claim 47 wherein the compound of Formula IV has the substituents indicated in Table 3 of the specification.

49. The method of claim 36, wherein the compound is selected from the following structures

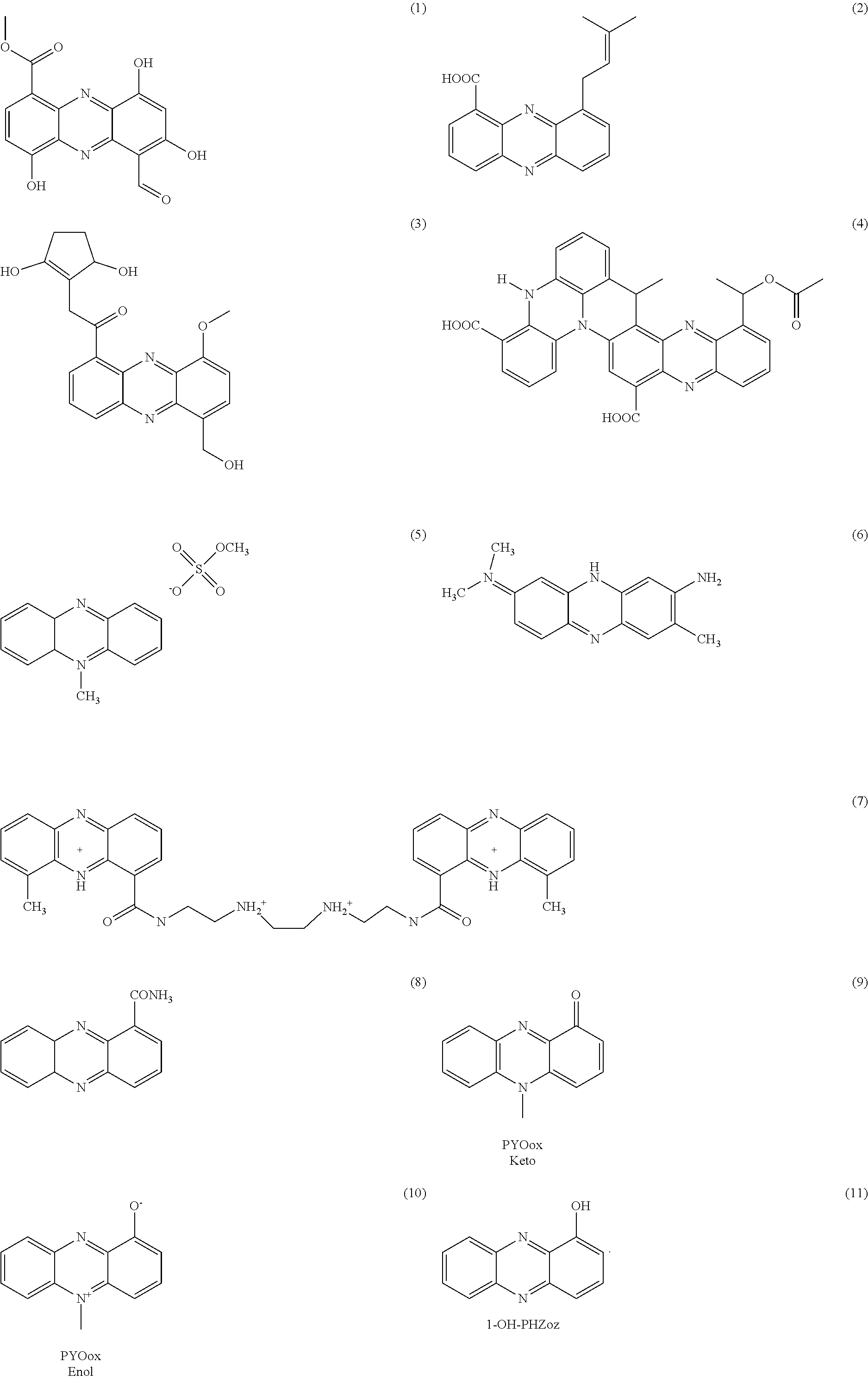

50. The method of claim 1, wherein the redox active bacteria comprise one or more bacteria of the order Pseudomonales, Burkholderiales Xanthomonadales, Burkholderiales Enterobacteriales, Streptomycetales, Pseudonocardiales, Micromonosporales, Streptosporangiales, Corynebacteriales, and/or Micrococcales.

51. The method of claim 1, wherein the redox active bacteria comprise one or more bacteria of genera Streptomyces, Pseudomonas Staphylococcus, Klebsiella Enterobacter, Escherichia Brevibacterium and/or Mycobacteria.

52. The method of claim 1, wherein the redox active bacteria comprise Pseudomonas, Coryneform Bacteria, Nocardia Brevibacterium linens, Brevibacterium, Burkholderia cenocepecia, Methanosarcina mazei, Mycobacterium abscessus, Pantoea agglomerans, Pectobacterium atrosepticum, Pelagio variabilis, Pseudomonas fluorescens, Streptomyces anulatus, Streptomyces cinnamonensis, and/or Shewanella onidensis.

53. The method of claim 1, wherein the redox active bacteria comprise Staphylococcus aureus, P. aeruginosa, P. oryzihabitans, and P. luteola, and/or Burkholderia cepacian.

54. The method of claim 1, wherein the medium is an inert surface of an environment outside an individual.

55. The method of claim 1, wherein the medium is selected from an organ, a tissue and/or a fluid from an individual.

56-57. (canceled)

58. The method of claim 55, wherein the medium is a wound selected from an acute wound and a chronic wound.

59. (canceled)

60. The method of claim 1, further comprising
contacting the redox active bacteria with one or more antibiotic and/or other antimicrobial for a time when the concentration of the redox active compound in the reduced state is higher than the concentration of the redox active compound in the oxidized state thus further inhibiting viability of the redox active bacteria in the medium.

61. The method of claim 60, wherein the antibiotics are in a sub-MIC amount.

62. The method of claim 60, wherein the antibiotic comprises one or more aminoglycosides.

63. The method of claim 62, wherein the antibiotic comprises one or more of an aminoglycoside of 4,6-disubstituted deoxystreptamine sub-class of aminoglycosides, an aminoglycoside of 4,5-disubstituted sub-class, and a non-deoxystreptamine aminoglycoside.

64. The method of claim 60, wherein the antibiotic comprises one or more of Kanamycin A Amikacin, Tobramycin, Dibekacin, Gentamicin, Sisomicin, Netilmicin, Neomycins B, C, Streptomycin and Plazomicin.

65-68. (canceled)

69. The method of claim 1, wherein the operating and the contacting are performed under anaerobic conditions.

70. The method of claim 1, wherein the redox active bacteria is comprised in a biofilm.

71-90. (canceled)
